(19) United States
(12) Patent Application Publication
(10) Pub. No.: US 2014/0256613 A1
(43) Pub. Date: Sep. 11, 2014

(54) PEPTIDE COMPOSITIONS THAT DOWNREGULATE TLR-4 SIGNALING PATHWAY AND METHODS OF PRODUCING AND USING SAME

(71) Applicant: The Board of Regents of the University of Oklahoma, Norman, OK (US)

(72) Inventor: Shanjana Awasthi, Edmond, OK (US)

(73) Assignee: The Board of Regents of the University of Oklahoma, Norman, OK (US)

(21) Appl. No.: 14/213,374

(22) Filed: Mar. 14, 2014

Related U.S. Application Data
(63) Continuation-in-part of application No. 14/015,144, filed on Aug. 30, 2013, now abandoned.
Application No. 14/015,144 is a division of application No. 13/289,820, filed on Nov. 4, 2011, now Pat. No. 8,623,832.
(60) Provisional application No. 61/782,380, filed on Mar. 14, 2013.
(60) Provisional application No. 61/410,077, filed on Nov. 4, 2010.
(60) Provisional application No. 61/469,202, filed on Mar. 30, 2011.

(51) Int. Cl. C07K 14/705; A61K 38/17; A61K 45/06

(52) U.S. Cl. CPC C07K 14/7056; A61K 45/06; C07K 14/705; A61K 38/177; A61K 38/178
USPC 514/2.3; 514/15.5; 514/21.4; 514/21.5; 514/21.6; 514/21.7; 514/21.8; 514/16.5

(57) ABSTRACT

Peptide compositions are disclosed that include fragments of surfactant protein-A, or a derivative thereof, wherein the fragment binds to TLR4. Methods of producing and using the peptide compositions are also disclosed.

### CROSS REFERENCE TO RELATED APPLICATIONS/INCORPORATION BY REFERENCE STATEMENT

The present application claims benefit under 35 USC 119(e) of U.S. Ser. No. 61/782,380, filed Mar. 14, 2013. This application is also a continuation-in-part of U.S. Ser. No. 14/015,144, filed Aug. 30, 2013; which is a divisional of U.S. Ser. No. 13/289,820, filed Nov. 4, 2011, now U.S. Pat. No. 8,623,832, issued Jan. 7, 2014; which claims benefit under 35 U.S.C. 119(e) of provisional patent applications U.S. Ser. No. 61/410,077, filed Nov. 4, 2010; and U.S. Ser. No. 61/469,202, filed Mar. 30, 2011. The entire contents of the above-referenced patent applications are hereby expressly incorporated herein by reference.

### STATEMENT REGARDING FEDERALLY SPONSORED RESEARCH OR DEVELOPMENT

Not Applicable.

### BACKGROUND

The pathogen-pattern recognition receptors (PPRRs) are important components of innate immunity that sense the pathogenic stimuli and regulate host immune responses. Surfactant protein-A (SPA) and Toll-like receptor-4 (TLR4) have been identified as important PPRRs. TLR4 is expressed as a transmembrane receptor and is known as a “Signaling-PPRR”. On the other hand, SPA is synthesized by type II lung epithelial cells and secreted in the alveoli as a component of surfactant. SPA is known as a “Secretory-PPRR.” It has been demonstrated by the inventor and others that SPA constitutes the majority of surfactant proteins (SPs) and plays a critical role in the clearance of pathogens and downregulation of the inflammatory response. On the other hand, TLR4 recognizes pathogen or pathogen-derived ligands and endogenous stress proteins, and induces inflammatory and adaptive immune responses. In a number of diseases, including but not limited to lung inflammatory conditions, an exaggerated activation of TLR4 has been found associated with NF-κB and pro-inflammatory cytokine response.

Published reports suggest that the bronchoalveolar lavage pools (extracellular pools) of SPA are significantly reduced in lungs of infected patients and animal models. In contrast, TLR4 expression is increased. The reduction in the amounts of SPA, and simultaneous increase in TLR4 expression corroborates well with the clinical condition of patients having fulminant infection and inflammation, respectively. In these clinical scenarios, the introduction of SPA should facilitate clearance of pathogens and attenuate inflammation. However, currently-available clinical surfactants (used for improving lung function and maturity in pre-term infants) do not contain SPA or SP-D because it is difficult to mix large hydrophilic SPA proteins with lipids. As with any large protein, rapid clearance of large proteins, degradation and a non-specific immune response have also hampered the development of clinical surfactant having SPA.

Inflammatory Bowel Disease (IBD) causes chronic inflammation in the intestine and accounts for a huge economic cost associated with multiple clinic visits and hospitalizations. Therapeutic efficacy with currently recommended drugs has been limited because of toxic effects, nonspecific downregulation of overall immunity and increased risk of infection. Contemporary understanding suggests that activation of Toll-like receptor-4 (TLR4) and TLR4-nuclear factor (NF)-kappa B signaling in the gut causes an overproduction of inflammatory cytokines and trafficking of leukocytes, thus leading to uncontrolled intestinal inflammation. Moreover, persistent inflammation can lead to carcinogenesis. Thus, new therapies targeting TLR4 may be of clinical utility in these conditions.

Interestingly, recently published reports suggested that SPA directly binds to TLR4. However, the in vivo evidence of such an interaction has been lacking, and its functional relevance has not been fully elucidated.

Therefore, there is a need in the art for an understanding of the functional relationship of TLR4 and SPA, as well as compositions that interact in and/or inhibit said interaction and thereby block TLR4 signaling. It is to said compositions, as well as methods of producing and using same, that the presently disclosed inventive concepts are directed.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 illustrates the characterization of purified native baboon lung SPA. (A): Purified baboon SPA (5 μg in each lane) protein was run under reducing (+heating, +DTT) and partially-reducing (+heating, no DTT) conditions on SDS-PAGE gel and stained. (B): Immunoreactivity of purified SPA by western blotting with SPA-specific antibody (IB-SPA). The SPA protein was run on reducing SDS-PAGE gel prior to western blotting. (C): HPLC chromatogram of purified lung SPA.

FIG. 2 depicts the morphology and phenotype of human KG-1-cells-derived dendritic cells (DCs), primary adult baboon lung DCs and fetal baboon lung DC-precursor cells. Photomicrographs of wet mount of KG-1-derived DCs after days (A): 5 and (B): 13, of in vitro culture in presence of recombinant human-GM-CSF, IL4 and TNF-α. Flow-cytometric histogram charts of (C): KG-1-derived DCs on days 5 (dark line) and 13 (faded line), (D): adult baboon lung DCs, and (E): fetal baboon lung DC-precursor cells. The cells were stained with DC-markers-specific, fluorescent-conjugated antibodies or isotype control antibody (black area). The cells with high forward scatter (FSC) and side scatter (SSC) were gated, and histogram charts were obtained. The percent number and mean fluorescent intensity (MFI) values of DC-marker positive KG-1-derived DCs are shown in tabulated form. The percent number of primary lung DC or DC-precursor cells positive for DC-markers (DC) is shown within the chart itself. Values shown within parenthesis indicate MFI values. The percent number and MFI values of isotype control (iso) stained cells in M1 region are also shown. The results presented here are representative of at least three experiments.

FIG. 3 illustrates basal TLR4 expression by (A) primary adult baboon lung DCs, (B) KG-1-derived DCs, and (C) fetal baboon lung DC-precursor cells under steady-state conditions. Cell-surface expression of TLR4 was detected by flow cytometry after staining the cells with TLR4-specific antibody. The percent number and MFI values of cells stained with TLR4-specific antibody (TLR4) are compared with isotype control antibody-stained cells (I) in selected region (−). (D): Western blot showing undetectable expression of TLR4 in 5 μg cell lysate protein of KG-1-derived DCs (KG1-DC). An equal amount of cell lysate protein of HEK293 cells stably transfected with TLR4 (HEK-TLR4) served as positive control.

FIG. 4 depicts the localization of exogenously-added recombinant TLR4-MD2 protein by confocal microscopy and flow-cytometry. Confocal microscopic images of KG-1-derived DCs pulsed with ALEXA FLUOR® 594-conjugated recombinant TLR4-MD2 protein for (A) 1 hour and (B) 4 hours. Vybrant DiO (green) dye stains the cytoplasm, and Hoechst 33342 (blue) dye stains the nucleus of the cell. The images were acquired using 63× objective. (C) Flow-cytometric charts of KG-1-derived DCs pulsed with ALEXA FLUOR® 495-conjugated recombinant TLR4-MD2 protein after 1 hour (dark line) and 4 hours (dotted line). The histogram chart of non-pulsed cells (negative control) is shown under the black area. Cells were gated in M region. Percent number of cells (and MFI values) positive for fluorescence are shown within the chart. Results are representative of two experiments.

FIG. 5 graphically depicts the effect of purified native SPA, recombinant TLR4-MD2 protein and MD2 protein on phagocytic function of KG-1-derived DCs. (A): Confocal microscopic images of KG-1-derived DCs incubated with pHrodo-labeled E. coli bioparticles for 3 hours. Phagocytosed bioparticles fluoresce red. Cells without phagocytosed particles and extracellular bacteria do not fluoresce. Enlarged images of a cell (shown as circle) are also shown in the figure, at different z-stack slices. (B): The extracellular bacteria that are either settled at the bottom or lie towards the top do not emit any fluorescence. These images confirm that fluorescence is of phagocytosed bioparticles. Next, KG-1-derived DCs were incubated with (C): purified baboon lung SPA (0.2 and 2 μM); (D): recombinant TLR4-MD2 protein (0.06-0.6 μM) and functional-grade anti-human TLR4 antibody (HTA 125 clone, lmgenex, CA; control reaction); (E): recombinant MD2 protein (0.02-0.2 μM); and (F): purified baboon lung SPA (2 μM) and TLR4-MD2 protein (0.6 μM), for an hour prior to addition of pHrodo-labeled E. coli bioparticles. The phagocytic uptake of E. coli bioparticles was measured spectrofluorometrically at 550 nm excitation and 600 nm emission wavelengths. Results are mean (SEM) of three different experiments. *p<0.05 or ns: not significant as compared to basal phagocytosis.

FIG. 6 graphically depicts the effect of simultaneous addition of purified SPA and recombinant TLR4-MD2 protein on phagocytic function of primary (A) adult baboon lung DCs and (B) fetal baboon lung DC-precursor cells. The DCs were incubated with respective proteins for an hour prior to addition of pHrodo-labeled E. coli bioparticles. The phagocytic uptake of E. coli bioparticles was measured spectrofluorometrically. * p<0.05, ns: not significant or otherwise indicated. Results are mean (SEM) of three different experiments performed at different times.

FIG. 7 graphically depicts the effect of purified native SPA and recombinant TLR4-MD2 proteins on TNF-α secretion by DCs against E. coli. (A) Primary adult baboon lung DCs or (B) fetal baboon lung DC-precursor cells were incubated with effector molecules for an hour prior to addition of pHrodo-labeled E. coli bioparticles. After 3 hours incubation at 37° C. in 5% CO₂ incubator, the cell-free supernatants were collected and subjected to ELISA for measurement of TNF-α. The results are representative of two experiments performed separately in triplicate. * p<0.05, ** p<0.001, ns: not significant.

FIG. 8 illustrates the synthetic peptides derived from C-terminal CRD region of human-SPA. The peptides sequences and their location in SPA are shown within the figure. Underlined amino acids were recognized at the interface of SPA-TLR4 complex in the in silico analysis (FIGS. 12 and 14).

FIG. 9 illustrates (A) Immunoblotting of immunoprecipitates (IP-SPA and IP-TLR4) with anti-human SPA (IB-SPA) and TLR4 (IB-TLR4) antibodies, respectively, to confirm the immunoprecipitation of specific proteins from baboon lung. IP-SPA, IP-TLR4, and adult baboon lung homogenate protein (40 μg) were run on 8% SDS-PAGE gel under nonreducing (no heating, no DTT) or partially reducing (+heating, no DTT) or reducing (+heating, +DTT) condition. (B) SYPRO-ruby-stained SDS-PAGE gel of IP-SPA run under partially reducing (+heating, no DTT) condition. Estimated molecular weights of major protein bands are shown within the gel-image. Expected locations of SPA, TLR4, and MD2 proteins are also marked. (C) Cross-immunoblotting of IP-SPA and IP-TLR4 with anti-human-TLR4 (IB-TLR4) and SPA (IB-SPA) antibodies, respectively. Purified SPA protein and lysate protein of HEK293 cells stably-transfected with TLR4 (HEK293-TLR4) served as positive control. (D) Negative controls for immunoprecipitation reaction: lanes 1, 2, 9, 10: IP-SPA and IP-TLR4 immunoblotted with nonspecific primary antibody; lanes 3, 4, 11, 12: IP-SPA and IP-TLR4 without any antigen or lung tissue homogenate; lanes 5, 6: 1.5 and 1 μl SPA antibody, respectively; lanes 13, 14: 1.5 and 1 μl TLR4 antibody, respectively; lanes 7, 8, 15, 16: IP reactions in absence of immunoprecipitating antibodies in the columns. The numbers indicate molecular weight (kDa) of standard marker proteins.

FIG. 10 graphically illustrates the binding between SPA and recombinant-TLR4-MD2 or MD2 protein by a microwell based-method. Various concentrations of (A) lung tissue homogenate protein (0.2-2 mg/ml) or (B) purified SPA protein (2.5-40 μg/ml) were incubated with immobilized recombinant TLR4-MD2 protein (0.25 μg per well) and the complex was detected using SPA-specific antibody. (C) Binding between purified baboon lung SPA and immobilized recombinant MD2 protein (0.25 μg per well). Various concentrations of purified SPA protein (2.5-100 μg/ml) were added. The wells were washed and the complex was detected using SPA-specific antibody. The binding of SPA to BSA protein shows non-specific binding. The results are representative of two experiments performed in triplicate. The error bars represent standard error of mean (SEM). * p<0.05, †p<0.1 versus BSA control (t-test).

FIG. 11 depicts a comparison of SPA, TLR4 and MD2 amino acid sequences of different animal species. Alignment of amino acid sequences of (A) TLR4, (B) MD2, and (C) SPA proteins in rat, mouse, baboon, macaca, and human. The X-ray crystal structures of human TLR4, human MD2 and rat SPA available in PDB format were used for bioinformatics simulations (FIGS. 11 and 12). The amino acid residues of SPA, TLR4 and MD2 included in the bioinformatics simulations are shown (←start, →end). Homology between the proteins of different species is shown as *. FIG. 11A: mouse TLR4, SEQ ID NO:247; rat TLR4, SEQ ID NO:248; baboon TLR4, SEQ ID NO:249; and human TLR4, SEQ ID NO:250. FIG. 11B: macaca MD2, SEQ ID NO:251; human MD2, SEQ ID NO:252; mouse MD2, SEQ ID NO:253; and rat MD2, SEQ ID NO:254. FIG. 11C: rat SPA, SEQ ID NO:255; mouse SPA, SEQ ID NO:256; baboon SPA, SEQ ID NO:257; and human SPA, SEQ ID NO:1.

FIG. 12 illustrates that the c-terminal portion of SPA binds to the extracellular domain of the TLR4-MD2 complex. First, the structure of SPA trimer was predicted by the SymmDock program from the monomeric crystal structure (PDB ID 1R13). Next, the predicted trimer was used to dock with TLR4-MD2 complex (PDB ID 3FXI) using GRAMM-X webserver. The above configuration is the most likely interaction model, based on GRAMM-X server ranking and detailed analysis.

FIG. 13 illustrates the amino acids that are likely to interact in the docked model of SPA-TLR4-MD2 complex, as shown in FIG. 12. In the illustration here, the other parts of the complex (two chains of SPA and TLR4) are rendered transparent to focus on the SPA-MD2 interaction site.

FIG. 14 illustrates the docked model of SPA-TLR4-MD2 complex, as shown in FIG. 12. This illustration shows that SPA interacts with TLR4 in SPA-TLR4-MD2 complex in at least four different places. The second monomer of the TLR4-MD2 dimer has been removed from the original model here for clarity. Also, the non-interacting chains of SPA and MD2 molecule have been rendered transparent.

FIG. 15 graphically depicts the effect of synthetic SPA peptides on LPS stimulated-TNF-α release by JAWS II dendritic cells. The experimental schematic is shown for pre-LPS and post-LPS treatment of cells with SPA-peptides. The control cells were treated with vehicle control, SPA-peptides (1 and 10 μM) or LPS (75 ng/ml) alone for 5 hours. The cell-free supernatants were collected after 5 hours of stimulation. The results are from three experiments performed in triplicate. The error bars represent SEM. *p<0.05 and ** p<0.001 as compared to TNF-α levels in cell-free-supernatants of LPS-treated cells (Analysis of variance (ANOVA)).

FIG. 16 graphically depicts the binding between SPA4 peptide and recombinant-TLR4-MD2 protein by a microwell based-method. Native SPA purified from baboon lung was included as control. Various amounts of purified native SPA protein (2-10 μg) or SPA4 peptide (2-20 μg) were incubated with immobilized recombinant TLR4-MD2 protein (0.25 μg per well), and the complex was detected using SPA-specific antibody. The results are from one representative experiment of three experiments performed in triplicate. The error bars represent SEM. The binding of SPA or SPA4 peptide to BSA protein shows non-specific binding. *p<0.05 as compared to 0 μg protein (t-test).

FIG. 17 depicts that SPA4 peptide reduced LPS-induced TLR4 expression. Rhodamin-phallodin (red) stained actin, Hoechst 33342 (blue) stained nucleus, and Alex fluor 488 (green) stained TLR4.

FIG. 18 illustrates that SPA4 peptide reduces inflammation in Dextran sulfate sodium (DSS)-colitis model. DSS challenge induced the colitis symptoms (edema and thickening shown with arrow). Mice with colitis lost about 25% of the body weight, colon was distended and shortened, and serum had increased levels of circulating TNF-α. Simultaneous treatment with SPA4 peptide (100 μg daily) reduced the (A) colitis symptoms. (B) The body weights (* p<0.001, # p<0.01, ns=not significant) and (C) colon lengths recovered after simultaneous treatment with SPA4 peptide. (D) SPA4 peptide treatment completely inhibited the DSS-induced serum TNF-α.

FIG. 19(A) illustrates the amino acid sequence of the SPA4 peptide derived from the TLR4-interacting region of SPA. FIG. 19(B) contains a high pressure liquid chromatogram (HPLC chromatogram), while FIG. 19(C) contains a mass spectrum; FIGS. 19(B) and 19(C) indicate the purity of synthetic SPA4 peptide (Genscript, CA).

FIG. 20 illustrates the secondary structure and relative solvent accessibility (RSA) values for the 20mer SPA4 peptide (SEQ ID NO:3) as predicted by the Solvent AccesiBiLitiEs (SABLE) program (Division of Biomedical Informatics, Children Hospital Research Foundation, Cincinnati, Ohio). The values indicate RSA values of amino acids exhibiting >25% RSA values.

FIG. 21 illustrates binding of SPA4 peptide to LPS-TLR4-MD2. (A) SPA4 peptide does not bind to LPS as measured by Limulus Amoebocyte Lysate (LAL) assay. The assay reaction was read after 6 and 12 minutes of substrate-addition. An equivalent amount of polymyxin B was included as positive control. (B) Computer model of LPS-TLR4-MD2 showing the binding sites of LPS (within yellow shadowed area) and SPA4 peptide (shown within blue shadowed area). LPS, TLR4 and MD2 structures are shown in red, blue/green and grey colors, respectively. This figure is reprinted and adapted by permission from MacMillan Publisher Ltd. (Park et al. (2009) Nature, 458:1191-1195).

FIG. 22 illustrates the effect of SPA4 peptide on TLR4 expression. SW480 cells were challenged with LPS (100 ng/ml or 1.0 μg/ml) for 4 hours and treated with SPA4 peptide (1, 10, or 100 μM) for 1 hour. The cells were subsequently stained with ALEXA FLUOR® 488-labeled antibody specific for TLR4 (Molecular Probes, Inc., Eugene, Oreg.; green), nuclear stain Hoechst 33342 (blue), and cytoplasmic phalloidin stain (red). (A) Confocal images of representative fields are shown for untreated cells, cells treated with 1, 10, or 100 μM SPA4 peptide, cells challenged with 1 μg/ml LPS alone, and LPS-challenged cells treated with 1, 10, or 100 μM SPA4 peptide. (B) Confocal images of different fields were acquired for the cells in which both the nucleus and cytoplasm were visible in the same plane. The fluorescent staining for TLR4 was quantified by densitometry. Mean (±SEM) densitometric units are shown as bars. Results are from one out of three independent experiments performed at different times. * p<0.05 versus LPS-challenged cells (ANOVA).

FIG. 23 shows that SPA4 peptide inhibits LPS-induced NF-κB activity. The luciferase activity was measured in cell lysates harvested after 5 hours and 9 hours of short-term and long-term SPA4 peptide treatment models of 1.0 μg/ml LPS-challenged SW480 cells. Cells were co-transfected with either (A) pcDNA-vector and NF-κB-luciferase reporter plasmid DNAs or (B) MYD88-dominant negative (MYD88DN) and NF-κB-luciferase reporter plasmid DNAs. The bars indicate mean (SEM) luminescence values normalized with μg total cell lysate protein. The results are from one experiment representative of four independent experiments performed in triplicate. * p<0.05 and ns: not significant versus LPS-challenged cells.

FIG. 24 illustrates the effect of SPA4 peptide on the expression of NF-κB signaling molecules: IKBα, phosphorylated IKBα, p65, phosphorylated p65, RelB, and COX-2. Ten μg total cell lysate protein was run on 4-20% Tris-glycine SDS-PAGE gels under partially-reduced conditions (heating at 95° C. for 5 minutes, no reducing agent). Separated proteins were immunoblotted with antibodies specific to respective molecules. Images of immune complexes were acquired and bands were analyzed densitometrically. (A) Acquired images of immunoreactive bands of IKBα, phosphorylated IKBα, p65, phosphorylated p65, RelB, and COX-2 in SW480 cells treated with LPS±SPA4 peptide on short- and long-term basis (see FIG. 23). (B) Densitometric readings of particular immunoreactive molecule normalized with those of beta-actin (β-actin), which was included as a loading control. Results are from one out of two independent experiments.

FIG. 25 illustrates the inhibition of LPS-induced expression of cellular IL-1β and IL-6 cytokines by SPA4 peptide. SW480 cells were treated with SPA4 peptide in a post-LPS challenge model. Cell lysate proteins were probed with antibodies-specific to (A) IL-1β and (B) IL-6 cytokines. Densitometric readings of immunoreactive IL-1β and IL-6 bands were normalized with those of β-actin. β-actin served as a loading control. Results are from one experiment of two independent experiments.

FIG. 26 illustrates the inhibition of LPS-stimulated migration of SW480 cells by SPA4 peptide. (A) Photomicrograph images are shown from one representative of four independent experiments performed at different times. At the beginning of the experiment, a “reference line” (central dark line) and markers were drawn at the bottom of the plate. After scraping, the cells were treated with LPS±SPA4 peptide. On the 0 hour images, a “start line” was drawn to represent the starting points for cells. On 72 hours images, a second line was drawn along the edge of cells to represent the migration of cells. (B) Percent cell migration was calculated for LPS±SPA4 peptide-treated cells as compared to untreated control cells for each experiment. The bar chart is mean (SEM) of four independent experiments performed at different times. * p<0.05 versus LPS-challenged cells.

FIG. 27 illustrates the inhibition of lipopolysaccharide-stimulated invasion of SW480 cells by the SPA4 peptide. (A, B) SW480 cells were challenged with 1 μg/mL lipopolysaccharide (LPS) for 4 hours and then with SPA4 (1 and 10 μM). After incubation for 96 hours, the matrix was scrubbed off from the top of the insert, and the bottom of the insert was stained with Diff-Quick Wright-Giemsa stain. The photomicrographs of invading cells were taken using a 20× objective. (A) Representative photomicrographs of Wright-Giemsa stained, untreated control, SPA4 peptide-treated and LPS±SPA4 peptide-treated SW480 cells. (B) The numbers of invading cells were counted in at least 15 representative microscopic fields, and percentages of cell that invaded the Matrigel under various conditions were calculated relative to the ones obtained with LPS only. The dotted line indicates 100% invasion, as set for LPS-challenged cells. The bar chart represents means (±SE M) from one out of three independent experiments. **p<0.001 vs. LPS-challenged cells (ANOVA).

FIG. 28 illustrates the effect of SPA4 peptide on cell cycle progression of SW480 cells. SW480 cells were challenged with LPS (100 ng/ml) for 4 hours following the treatment with SPA4 peptide (10, 50 and 100 μM). After 40 hours of total incubation period, cells were harvested, stained with propidium iodide and run on a flow-cytometer. Cell cycle analysis was performed using ModFIT program. The results are from one experiment representative of four independent experiments. (A) Strategy to gate out the cell aggregates by plotting FL3-Width (W) versus FL3-Area (A) dot-plot chart. Single cells are shown within R region. (B) ModFIT program was used to de-convolute the populations of single cells in R region and percentage values of each population are indicated within the chart.

FIG. 29 shows that SPA4 peptide inhibits the viability of SW480 cells. SPA4 peptide inhibits the viability of SW480 cells. SW480 cells were challenged with LPS (100 ng/ml) for 4 hours following the treatment with SPA4 peptide (10, 50 and 100 μM). Cells were harvested after 3, 4 and 5 days of incubation, stained with propidium iodide and run on a flow-cytometer. (A) Percent numbers of dead cells are shown as cells staining positive for propidium iodide in marked region (M1) within histogram charts. The results are from one representative experiment. Unstained cells served as negative control for setting the gate. (B) Results shown here are mean (SEM) percent number of dead cells. Three independent experiments were performed at different times. * p<0.05, **p<0.001

FIG. 30 illustrates that SPA4 peptide inhibits endotoxic-shock like symptoms. Mice were challenged with LPS (0.1 microg/g body wt) via intraperitoneal route. Mice were then injected with SPA4 peptide (2.5 microg/g body wt) after 1 hour, 6 hours and 12 hours of LPS challenge or purified lung SPA (0.5 microg/g body wt) at 1 hour and 6 hours of LPS challenge. The symptoms (Ruffled fur, reactivity, eye exudate, diarrhea, breathing problem) were noted at the scale of 0-3 after 7 hours of LPS challenge. Mean symptom indices (SEM) are shown here for each treatment group.

FIG. 31A illustrates that SPA4 peptide inhibits LPS-induced TNF-α. Mice were challenged with LPS (0.1 microg/g body wt) via intraperitoneal route. Mice were then injected with SPA4 peptide (2.5 microg/g body wt) after 1 hour, 6 hours and 12 hours of LPS challenge or purified lung SPA (0.5 microg/g body wt) at 1 hour and 6 hours of LPS challenge. Mice were sacrificed after 26 hours of LPS challenge. Blood was collected at the time of sacrifice. TNF-α levels were measured in serum samples by ELISA method. Mean (SEM) TNF-α levels (pg/ml) are shown within each group. FIG. 31(B) illustrates that SPA4 peptide alleviates clinical symptoms in LPS-challenged mice. The time and doses of LPS-challenge and SPA or SPA4 peptide treatment are shown in flow chart format.

FIG. 32 illustrates that SPA4 peptide treatment reduces the LPS-induced TNF-α levels in lung tissue homogenates. Mice were challenged with 1 μg LPS per g body weight intraperitoneally at 0 hour and treated with SPA4 peptide (2.5 μg/g body wt) or SPA (Malt baboon SPA, 0.5 μg/g body wt) at 1 hour, and sacrificed at 5 hours. Lung tissues were harvested at the time of sacrifice. TNF-α levels were measured in lung tissue homogenates by ELISA, and normalized with total lung tissue homogenate protein. Mean (SEM) pg TNF-α amounts per mg total lung protein are shown within the bar chart.

FIG. 33 illustrates as follows: (A) (i) Expression of SPA and SPA-mutant proteins as fusion proteins with VP16, and TLR4-GAL4 fusion protein by transiently-transfected HEK293 cells. Ten μg of total cell lysate proteins were separated on 4-20% tris-glycine SDS-PAGE gradient gel under complete reducing (heating for 5 minutes+11 mM β-mercaptoethanol) or partially-reducing (heating for 5 minutes) condition and probed with SPA- or TLR4-specific antibody, respectively. Results in left panel indicate SPA-antibody reactive bands. Lanes 1: purified lung SPA (10 ng); 2: lysate protein from cells transfected with pSPA-mutant and pTLR4, 3: lysate protein from cells transfected with pSPA and pTLR4 plasmid DNA constructs, and 4: lysate protein from nontransfected cells. Results in right panel demonstrate the TLR4-antibody reactive bands. Lanes 1: lysate protein from cells transfected with pSPA-mutant and pTLR4, 2: lysate protein from cells transfected with pSPA and pTLR4 plasmid DNA constructs and 3: lysate protein from nontransfected cells. (ii) Secreted amounts of SPA in cell-free supernatants of nontransfected, pSPA+pTLR4 and pSPA-mutant+pTLR4 co-transfected HEK293 cells. SPA levels (in ng) normalized with total μg cellular proteins are listed. (iii) Cell-surface expression of TLR4 in nontransfected HEK293 cells, and in pSPA+pTLR4 and pSPA-mutant+pTLR4 co-transfected HEK293 cells. Percent number of TLR4-positive cells (T) in region R is shown within the histogram plots. Isotype control antibody-stained cells were included as control (IC). (iv) Confocal microscopy of the SPA- and TLR4-antibody stained nontransfected HEK293 cells (negative control). Nontransfected HEK293 cells were permeabilized, immunostained with antibodies against SPA (green) and TLR4 (red) and counterstained with nuclear dye (blue). Scale bar is shown within the image. (v) Expression of SPA (in green) and TLR4 proteins (in red) and their co-localization (in yellow) in HEK293 cells transfected with pSPA and pTLR4 by confocal microscopy. Cells were permeabilized and immunostained with antibodies against SPA and TLR4 proteins, and counterstained with nuclear dye (blue). The confocal images in the bottom panel are from a single HEK293 cell transfected with pSPA and pTLR4 plasmid DNA constructs. (B) Loss of SPA4 peptide region in SPA-mutant protein results into decreased Firefly luciferase activity in cells transfected with pSPA-mutant and pTLR4. Relative luminescence units (RLU) depicting interaction between SPA and TLR4 (100) and SPA-mutant protein and TLR4 by two-hybrid assay. Negative controls included HEK293 cells transfected with pACT and pBIND (vector backbones). Additional controls included were cells transfected with pSPA, pBIND and pcDNA3.0 and non-transfected cells (not shown). Positive control included cells transfected with pACT-MyoD and pBIND-ID plasmid DNA constructs (not shown). The error bars represent standard error of mean (SEM). Results are from four experiments performed separately at different times. Statistical significance (p value <0.007) is shown as compared to SPA-TLR4 interaction (t-test). (C) The NF-κB luciferase reporter activity was measured in HEK293 cells transfected with pSPA or pSPA-mutant and pTLR4 plasmid DNA constructs and challenged with LPS (100 ng/ml) for 5 hours. The Renilla luciferase activity was measured to assess the transfection efficiency. The luminescence values for NF-κB-associated Firefly luciferase activity normalized with those for Renilla luciferase activity are shown as bar chart. The bars show Mean+SEM of results from three separate experiments performed at different times. p<0.001 was noted as compared to the values in cells transfected with pTLR4 only (ANOVA).

FIG. 34 illustrates the primary chemical structure of SPA4 peptide as predicted by PepDraw Program (Tulane University, LA). Automated generated isoelectric point, net charge and extinction coefficient of SPA4 peptide are shown within the figure.

FIG. 35 illustrates (A) in silico predictions of 3D structure of SPA4 peptide as predicted by PEP-FOLD online server, and (B) a Kyte and Doolittle hydropathy index that shows negative values indicating the hydrophilic nature of the SPA4 peptide.

FIG. 36 illustrates the effect of post-LPS treatment with synthetic SPA4 peptide on phospho-NF-κB-p65 expression in JAWS II dendritic cells. The JAWS II dendritic cells were challenged with LPS (100 ng/ml) for 4 hours and subsequently treated with SPA4 peptide (1 μM and 10 μM) for 1 hour. Twenty μg of total cell lysate protein was separated on 4-20% tris-glycine SDS-PAGE gel under complete reducing condition (heating+11 mM β-mercaptoethanol) and immunoblotted with phospho-NF-κB-p65 antibody. Phospho-NF-κB-p65 and β-actin antibodies-reactive bands in lysate proteins of cells treated with (Lanes 1:) vehicle control, (2:) 1 μM SPA4 peptide, (3:) 10 μM SPA4 peptide, (4:) 100 ng/ml LPS, (5:) 100 ng/ml LPS+1 μM SPA4 peptide, and (6:) 100 ng/ml LPS+10 μM SPA4 peptide. The bar chart in the bottom panel demonstrates the densitometric ratio of phospho-NF-κB-p65 and β-actin antibody-reactive bands. The results from one representative experiment. Similar experiments were performed twice separately.

FIG. 37 illustrates the effect of post-LPS treatment with synthetic SPA4 peptide on NF-κB activity and TNF-α release in JAWS II dendritic cells. (A) The JAWS II dendritic cells were co-transfected with NF-κB-luciferase reporter and MYD88-dominant negative (MYD88DN) or pcDNA3.0 vector plasmid DNA constructs. Cells were stimulated with LPS (100 ng/ml) for 4 hours and subsequently treated with SPA4 peptide (1 and 10 μM) for 1 hour. The cells were lysed and NF-κB-associated luciferase activity was measured. Luminescence units were normalized with total cellular protein. Percent NF-κB luciferase reporter activity was compared to that in LPS-stimulated cells; p<0.01 or p<0.05 were noted (ANOVA); ns: not significant. (B) The TNF-α levels were measured in cell-free supernatants by ELISA and normalized with total cellular protein. p<0.05 as compared to TNF-α levels in cell-free-supernatants of LPS-treated cells (ANOVA); ns: not significant. The bars represent mean+SEM values obtained from three experiments performed in triplicate at different times.

FIG. 38 illustrates the effect of SPA4 peptide on LPS-induced circulating levels of (A) TNF-α and (B) endotoxic shock-like symptoms in a mouse model. Mice were challenged with LPS (15 μg per g body mass) at 0 hour, treated with SPA4 peptide (2.5 μg per g body mass) or purified lung SPA (0.5 μg per g body mass) at 1 hour and sacrificed at 6 hours post-LPS challenge. (A) TNF-α levels (pg/ml) were measured in serum samples of mice by ELISA. Results are shown as mean+SEM. (B) The endotoxic shock-like symptoms (ruffled fur, prostration, reactivity, diarrhea, and eye exudate) were noted for each mouse on the scale of 0-3 and given an average symptom index. Results are from 6 mice in control group and 10 mice each per LPS-challenged and SPA or SPA4 peptide treatment groups included in two separate experiments. Statistical significance was calculated by employing t-test.

FIG. 39 illustrates the effect of SPA4 peptide on LPS-induced lung TNF-α levels in a mouse model. Mice were challenged with LPS (15 μg per g body mass) at 0 hour, treated with SPA4 peptide (2.5 μg per g body mass) or purified lung SPA (0.5 μg per g body mass) at 1 hour and sacrificed at 6 hours post-LPS challenge. Harvested lung tissue specimens were homogenized, and TNF-α (in pg/ml) and total protein (in μg/ml) concentrations were measured in lung tissue homogenates by ELISA and BCA protein assay, respectively. The TNF-α levels were normalized with total lung protein amounts. The lines represent mean of results obtained from two experiments. Statistical significance was calculated by employing t-test.

FIG. 40 illustrates that SPA4 peptide treatment ameliorates the LPS-induced histological changes in lung. Mice were challenged with LPS (15 μg LPS per g body mass) at 0 hour, treated with SPA4 peptide (2.5 μg per g body mass) or purified lung SPA (0.5 μg per g body mass) at 1 hour and sacrificed at 6 hours post-LPS challenge. (A) Histological observations in lung tissue sections of untreated control mice: an occasional neutrophilic leukocyte (shown as arrow) was present within the pulmonary vessels. There was no evidence of significant neutrophilic leukocyte pavementing along the endothelial lining (400×, H&E stain); LPS-challenged mice: Numerous neutrophilic leukocytes were present within the lumen and observed pavementing (shown as arrows) along the endothelial lining of the pulmonary vessels (200×, 400× and 600×, H&E stain); LPS-challenged, SPA-treated mice: Numerous neutrophilic leukocytes were present within both the lumen (shown as arrows) and pavementing along the endothelial lining of the pulmonary vessel (400× and 600×, H&E stain); LPS-challenged, SPA4 peptide-treated mice: Only an occasional neutrophilic leukocyte was observed within the central lumen of the pulmonary vessel. There was only minimal evidence of neutophilic leukocyte pavementing (shown as arrow) along the endothelial lining. (400× and 600×, H&E stain). (B) Number of leukocytes counted per pulmonary vessels in mice groups as compared to the ones in LPS-challenged mice. The number of leukocytes per vessel in LPS-challenged mice group was set at 100% and relative percentages were calculated in other groups. Statistical significance was calculated by employing t-test.

FIG. 41 illustrates immunohistochemistry for endogenous expression and nuclear localization of NF-κB-p65 in lung tissues of untreated control, LPS-challenged±SPA4 peptide- or purified lung SPA-treated mice. Formalin-fixed lung tissue specimens were sectioned into 5 μm sections and stained with an antibody to NF-κB-p65 (shown in blue). Finally tissue sections were counterstained with nuclear fast red stain (shown in red). The photomicrographs were taken using 20× (upper panel) and 100× (bottom panel) objective lens. In the bottom panel, representative area within each photomicrograph is enlarged for better visualization. The arrows within the photomicrograph indicate nuclear localization of NF-κB-p65.

FIG. 42 shows mass spectrum and HPLC chromatogram of synthetic SPA4 and FITC-SPA4 peptides. Mass spectrum (theoretical molecular weight: 2397.48, observed molecular weight: 2396.90) of SPA4 peptide (A). The purity of SPA4 peptide was 96.1% as characterized by Alltima™ C18 HPLC (4.6×250 mm) (B). Mass spectrum (theoretical molecular weight: 2900.02, observed molecular weight: 2899.05) of FITC-SPA4 peptide (C). The purity of FITC-SPA4 peptide was 95.4% as characterized by YMC-Pack C4 HPLC (4.6×250 mm) (D).

FIG. 43 illustrates direct binding of FITC-SPA4 peptide to recombinant TLR4-MD2 protein as determined by fluorescence polarization assay. Two μM of FITC-SPA4 peptide was added to wells containing different concentrations of TLR4-MD2 protein. The background fluorescence polarization values were subtracted and plotted. The binding plot was then fitted into a one-site binding model using standard regression analysis and Kd values were noted. Results are from one representative experiment out of a total of three separate experiments performed at different times.

FIG. 44 illustrates expression of GFP protein by GFP-E. coli 19138 and GFP-P. aeruginosa 8830. Flow cytometric histogram charts demonstrating the expression of GFP by GFP-E. coli (A) and GFP-P. aeruginosa (B). Non-GFP expressing E. coli 19138 and P. aeruginosa PAO1 were included as controls. The values represent mean fluorescent intensities of non-GFP and GFP-bacteria are within the “M” region. Green fluorescence of GFP-E. coli and GFP-P. aeruginosa was also confirmed by confocal microscopy (shown in inset).

FIG. 45 illustrates that SPA4 peptide induces phagocytosis of live GFP-P. aeruginosa (A, B) and GFP-E. coli (C, D). Flow cytometric dot plot charts of dendritic cells (gated in region P), bacteria (gated in region R). The dot plot charts demonstrate the cells with phagocytosed bacteria in P1 area. Numbers indicate percent number of cells with GFP-E. coli or GFP-P. aeruginosa (in P1) or without any bacteria (in P2) (A, C; i-iv). Results are from one representative experiment out of three separate experiments. Representative confocal micrographs of cells alone or with phagocytosed GFP-P. aeruginosa (B) or GFP-E. coli (D). Images taken at brightfield and fluorescence channels were superimposed. Green fluorescence is of GFP-P. aeruginosa or GFP-E. coli, and blue staining is of cell nuclei.

FIG. 46 illustrates that SPA4 peptide treatment induces localization of bacteria inside acidic phagolysosomes of dendritic cells and alveolar macrophages, but suppresses the TNF-α response. Percent localization of pHrodo-labeled E. coli or P. aeruginosa in acidic phagolysosomes of dendritic cells (A, C) and alveolar macrophages (E) after treatment with SPA4 peptide. Tuftsin was included as positive control, and cytochalasin D was included as negative control. Bars represent mean+SEM of results from five (A, C) or two (E) separate experiments performed separately in triplicate. Secreted levels of TNF-α cytokine in cell-free supernatants of dendritic cells (B, D) and alveolar macrophages (F) exposed to pHrodo-labeled E. coli and P. aeruginosa. The p-values are shown within each figure for statistical significance. Confocal images show localization of red fluorescent pHrodo-labeled E. coli and P. aeruginosa inside the acidic phagolysosomes of dendritic cells (G, H) and alveolar macrophages (I). The cell nucleus stained with Hoechst 33342 dye is shown in blue. LAMP1 staining (in green) confirms the localization of pHrodo-labeled bacteria inside the LAMP1-expressing phagolysosome (J).

FIG. 47 illustrates the effect of SPA4 peptide on localization of bacteria inside the acidic phagolysosomes (A, C) and suppression of TNF-α levels (B, D) in cell-free supernatants of dendritic cells overexpressing TLR4. Cells were transfected with plasmid construct expressing wild-type TLR4 or vector control (pDisplay vector), exposed to pHrodo-labeled E. coli (A, B) and P. aeruginosa (C, D) and treated with SPA4 peptide. Red fluorescence due to internalized bacteria was quantitated by fluorometry and percent localization of bacteria was calculated relative to control; bars represent mean+SEM of results from three separate experiments. TNF-α levels were measured in cell-free supernatants by ELISA and normalized with total cellular protein. TNF-α results presented here are from one representative experiment performed in triplicate. The p-values are shown within each figure for statistical significance.

FIG. 48 illustrates that SPA4 peptide does not bind to live E. coli or P. aeruginosa. Live non-GFP E. coli and P. aeruginosa were incubated with 10, 50, 75, and 100 μM of FITC-SPA4 peptide and 75 μM of Oregon green (OG)-polymyxin B (positive control). No shift was observed in flow cytometric histograms of E. coli and P. aeruginosa in the FL1 channel when incubated with FITC-SPA4 peptide. In contrast, a significant shift is observed in flow cytometric histograms of bacteria in the FL1 channel when incubated with OG-polymyxin B, which binds to bacteria (A). Confocal microscopic images of live non-GFP bacteria incubated with FITC-SPA4 peptide (75 μM) and OG-polymyxin B (75 μM). The confocal images were obtained using brightfield and FL1 channels. Absence of fluorescence indicates no binding of FITC-SPA4 peptide to the bacteria. Green fluorescence indicates binding of OG-polymyxin B to the bacteria (B).

FIG. 49 illustrates that SPA4 peptide does not affect the growth of E. coli or P. aeruginosa. Growth curves (OD₆₀₀ versus time in hours) of E. coli (i) and P. aeruginosa (ii) cultured over 17 hours in the presence of increasing concentration of SPA4 peptide or vehicle. Ampicillin (3.5 μg/ml) and Kanamycin (100 μg/ml) antibiotics were used as positive controls for growth inhibition. Results were confirmed by colony counts (CFU/ml) obtained at 17 h.

FIG. 50 illustrates that SPA4 peptide treatment suppresses bacterial burden, inflammation, lung injury and alleviates clinical symptoms in a mouse model of P. aeruginosa lung infection. Flow chart depicts the schedule of challenge with live P. aeruginosa (1×10⁷ CFU as per the standard curve between OD₆₀₀ and CFU) and treatment with SPA4 peptide (50 μg) via the intratracheal route (A). Average symptom scores (B), lung wet weight (in g) (C), representative images of whole lung (D), bacterial burden (CFU/g lung) (E), cytokine (TNF-α) levels (pg/g lung) (F) were assessed after 5 hours of infectious challenge. Representative micrographs of H&E stained mouse lung tissues demonstrating foci of inflammatory cell influx (with 20× and 60× objectives) (G). The lung appears to be hypercellular related to a marked influx of inflammatory cells (arrows) consisting primarily of neutrophilic leukocytes. Inflammatory cells often appeared to be located within the alveolar sacs and interstitial areas (i, ii). Lungs from SPA4 peptide treated mice had an essentially normal appearance with only occasional neutrophilic leukocyte (arrow) being observed (iii, iv). Results presented are from one experiment (n=5 mice per group per experiment) and are representative of four experiments performed at different times. The p values are shown within each figure for statistical significance.

FIG. 51 graphically illustrates bacterial burden and cytokine levels in lung after bacterial challenge and treatment with SPA4 peptide with or without CUROSURF®. Mice were challenged with P. aeruginosa PAO1 (1.68×10⁷ CFU) intratracheally at 0 hours and treated with CUROSURF® (1.6 mg per mouse), or CUROSURF® (equivalent amount)+SPA4 peptide (50 μg), intratracheally at 1 hour. Mice were sacrificed at 5 hours. Whole lungs were harvested, minced, and plated on agar plates for bacterial counts. Minced lung tissue homogenates were centrifuged. The protease inhibitors were added to the supernatants before storing them at −80° C. The cytokines (TNF-α and IL-1β) were measured by ELISA and normalized per g lung wet weight. Results are derived from 5 mice in each group in an experiment.

FIG. 52 contains photomicrographs of Hematoxylin and Eosin (H&E) stained lung tissue sections following bacterial challenge with or without treatment with CUROSURF® and SPA4. Representative images of H&E stained lung tissue sections. In this experiment, mice were challenged with 1.93×10⁷ CFU intratracheally at 0 hours and treated with CUROSURF® (1.6 mg per mouse), or CUROSURF® (equivalent amount)+SPA4 peptide (50 μg), intratracheally at 1 hour. Mice were sacrificed at 5 hours, and whole lungs were harvested. The lungs were fixed in 10% formalin for 20-24 hours, transferred into 70% ethanol, sectioned, and processed for H&E staining. Leukocyte influx in the lung tissue sections is shown as black arrows.

### DETAILED DESCRIPTION

Before explaining at least one embodiment of the inventive concepts in detail by way of exemplary drawings, experimentation, results, and laboratory procedures, it is to be understood that the inventive concepts are not limited in its application to the details of construction and the arrangement of the components set forth in the following description or illustrated in the drawings, experimentation, and/or results. The inventive concepts are capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concepts shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those skilled in the art to which this presently disclosed inventive concepts pertain. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the presently disclosed inventive concepts have been described in terms of preferred embodiments, it will be apparent to those of skill in the art that variations may be applied to the compositions and/or methods and in the steps or in the sequence of steps of the method described herein without departing from the concept, spirit, and scope of the presently disclosed inventive concepts. All such similar substitutes and modifications apparent to those skilled in the art are deemed to be within the spirit, scope, and concept of the inventive concepts as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

Throughout the specification and claims, unless the context requires otherwise, the terms “substantially” and “about” will be understood to not be limited to the specific terms qualified by these adjectives/adverbs, but will be understood to indicate a value includes the inherent variation of error for the device, the method being employed to determine the value and/or the variation that exists among study subjects. Thus, said terms allow for minor variations and/or deviations that do not result in a significant impact thereto. For example, in certain instances the term “about” is used to indicate that a value includes the inherent variation of error for the device, the method being employed to determine the value and/or the variation that exists among study subjects. Similarly, the term “substantially” may also relate to 80% or higher, such as 85% or higher, or 90% or higher, or 95% or higher, or 99% or higher, and the like.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism. A molecule can be biologically active through its own functionalities, or may be biologically active based on its ability to activate or inhibit molecules having their own biological activity.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” as used herein includes human and veterinary subjects. “Mammal” for purposes of treatment refers to any animal classified as a mammal, including human, domestic and farm animals, nonhuman primates, and any other animal that has mammary tissue.

“Treatment” refers to both therapeutic treatment and prophylactic or preventative measures. Those in need of treatment include, but are not limited to, individuals already having a particular condition or disorder as well as individuals who are at risk of acquiring a particular condition or disorder (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, reduction in occurrence, prevention, or management of a disease and/or cancer. The specific amount that is therapeutically effective can be readily determined by the ordinary medical practitioner, and can vary depending on factors known in the art, such as the type of disease/cancer, the patient's history and age, the stage of disease/cancer, and the co-administration of other agents.

A “disorder” is any condition that would benefit from treatment with the polypeptide. This includes chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

The term “effective amount” refers to an amount of a biologically active molecule or conjugate or derivative thereof sufficient to exhibit a detectable therapeutic effect without undue adverse side effects (such as toxicity, irritation and allergic response) commensurate with a reasonable benefit/risk ratio when used in the manner of the inventive concepts. The therapeutic effect may include, for example but not by way of limitation, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy”, and will be understood to mean that the patient in need of treatment is treated or given another drug for the disease in conjunction with the pharmaceutical compositions of the presently disclosed inventive concepts. This concurrent therapy can be sequential therapy, where the patient is treated first with one drug and then the other, or the two drugs are given simultaneously.

The terms “administration” and “administering” as used herein will be understood to include all routes of administration known in the art, including but not limited to, oral, topical, transdermal, parenteral, subcutaneous, intranasal, mucosal, intramuscular, intraperitoneal, intravitreal and intravenous routes, including both local and systemic applications. In addition, the compositions of the presently disclosed inventive concepts (and/or the methods of administration of same) may be designed to provide delayed, controlled or sustained release using formulation techniques which are well known in the art.

The presently disclosed inventive concepts also include a pharmaceutical composition comprising a therapeutically effective amount of at least one of the compositions described herein in combination with a pharmaceutically acceptable carrier. As used herein, a “pharmaceutically acceptable carrier” is a pharmaceutically acceptable solvent, suspending agent or vehicle for delivering the compositions of the presently disclosed inventive concepts to the human or animal. The carrier may be liquid or solid and is selected with the planned manner of administration in mind. Examples of pharmaceutically acceptable carriers that may be utilized in accordance with the presently disclosed inventive concepts include, but are not limited to, PEG, liposomes, ethanol, DMSO, aqueous buffers, oils, DPPC, lipids, other biologically-active molecules, vaccine-adjuvants, and combinations thereof. The pharmaceutically acceptable carrier may be directly or indirectly associated with the peptides of the presently disclosed inventive concepts; for example but not by way of limitation, the pharmaceutically acceptable carrier may be directly attached to the peptide so as to form a conjugate (i.e., a PEGylated peptide), or the peptide may be indirectly associated with the carrier via disposal therein (i.e., liposomes, vesicles, buffers, oils, etc.).

In certain embodiments, the duration of action of the peptides of the presently disclosed inventive concepts may be controlled and/or enhanced by incorporation of the peptide into particles of a polymeric material, such as but not limited to, polyesters, polyamides, polyamino acids, hydrogels, poly(lactic acid), ethylene vinylacetate copolymers, copolymer micelles of, for example, polyethylene glycol (PEG) and poly(l-aspartamide), and the like. The peptide may also be ionically, covalently, or otherwise conjugated to the macromolecules described above, particularly PEG of various molecular weights. In certain embodiments, the peptides may be covalently linked at a suitable functional group such as the N-terminal end thereof to one or more PEG molecules to form a “PEGylated” peptide. Examples of PEG molecules that can be used include, but are not limited to, a “mini-PEG™” molecule comprising AEEA and/or AEEEA. AEEA is [2-(2-amino-ethoxy)-ethoxy]-acetic acid (also known as 8-Amino-3,6-Dioxaoctanoic acid), and AEEEA is {2-[2-(2-amino-ethoxy)-ethoxy]-ethoxy}-acetic acid (also known as 11-Amino-3,6,9-Trioxaundecanoic acid). The PEG molecule, for example, may have a molecular weight in a range of from about 350 Daltons to about 20,000 Daltons. More particularly, the PEG molecule may have a MW in a range of from about 450 Da to about 15,000 Da. More particularly, the PEG molecule may have a MW in a range of from about 1,000 Da to about 12,000 Da. More particularly, the PEG molecule may have a MW in a range of from about 2,000 Da to about 10,000 Da. More particularly, the PEG molecule may have a MW in a range of from about 3,000 Da to about 8,000 Da.

The terms “liposome,” “lipid nanostructure,” and “vesicle” may be used interchangeably herein and will be understood to refer to an assembled structure constructed of molecules such as lipids and/or proteins, for example, not through covalent bonds but through interactions (such as but not limited to, hydrophobic interactions, electrostatic interactions and hydrogen bonds) acting between the molecules in an aqueous medium.

The terms “aqueous solution” and “aqueous medium” will be used interchangeably herein and will be understood to refer to water as well as any kind of solution which is physiologically acceptable and solvent in water.

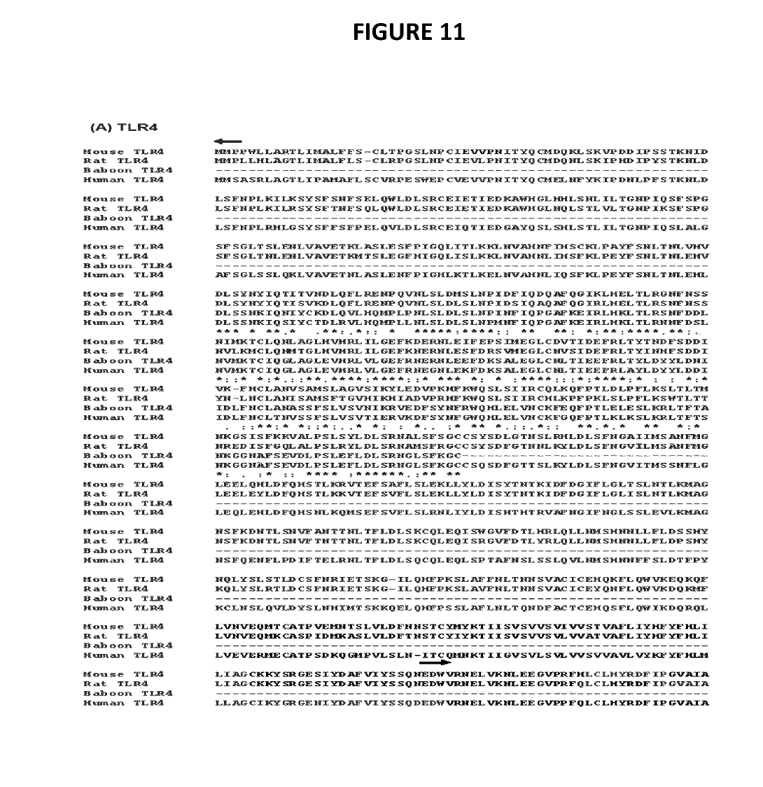

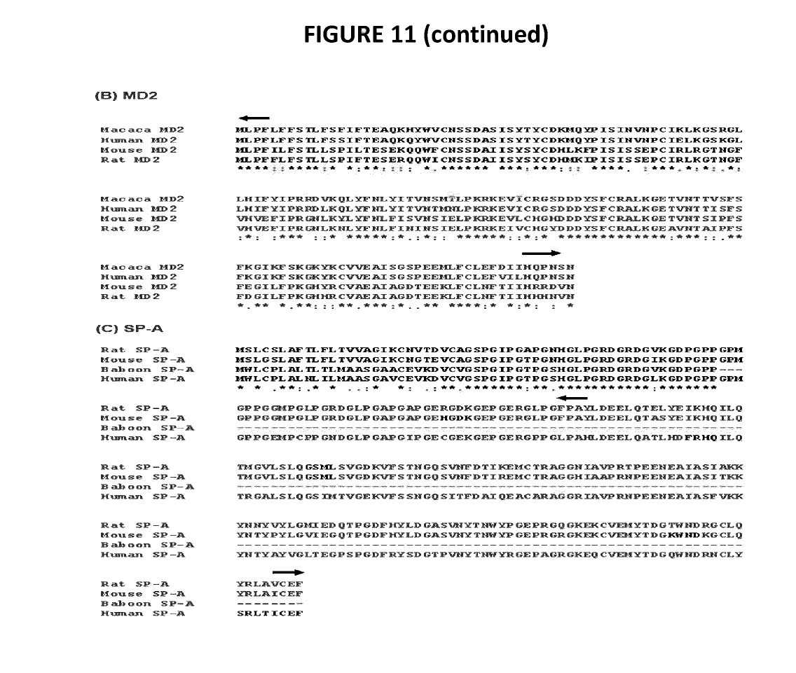

In certain embodiments, the presently disclosed inventive concepts are directed to a composition comprising an isolated peptide that comprises a portion of Surfactant protein A (SEQ ID NO:1 or any of the sequences shown in FIG. 11C). In certain embodiments, the isolated peptide comprises a portion of the C-terminal carbohydrate recognition domain of SPA; in particular embodiments, the isolated peptide comprises the following motif: NYTX₃₋₉RG (SEQ ID NO:2). In addition, the isolated peptide may be less than 50 amino acids in length (such as but not limited to, less than 49, less than 48, less than 47, less than 46, less than 45, less than 44, less than 43, less than 42, or less than 41 amino acids in length). In other embodiments, the isolated peptide may be less than 40 amino acids in length (such as but not limited to, less than 39, less than 38, less than 37, less than 36, less than 35, less than 34, less than 33, less than 32, or less than 31 amino acids in length). In other embodiments, the isolated peptide may be less than 30 amino acids in length (such as but not limited to, less than 29, less than 28, less than 27, or less than 26 amino acids in length). In yet other embodiments, the isolated peptide may be less than 25 amino acids in length (such as but not limited to, less than 24, less than 23, less than 22, or less than 21 amino acids in length). In still further embodiments, the isolated peptide may be less than 20 amino acids in length (such as but not limited to, less than 19, less than 18, less than 17, less than 16, less than 15, less than 14, less than 13, less than 12, less than 11, or less than 10 amino acids in length). In yet still further embodiments, the isolated peptide may be less than 10 amino acids in length (such as but not limited to, less than 9, less than 8, less than 7, less than 6, or less than 5 amino acids in length).

Said compositions of the presently disclosed inventive concepts are capable of binding to Toll-like receptor-4 (TLR4) and inhibiting TLR4 signaling pathway(s).

In one embodiment, the isolated peptide may comprise any of SEQ ID NOS:3-7, or a fragment thereof. For example but not by way of limitation, Table 1 lists SEQ ID NOS:8-246, which are exemplary fragments of SEQ ID NOS:3 and 5.

In certain embodiments, the presently disclosed inventive concepts are also directed to a composition that includes an isolated peptide that is a mutant or derivative of a portion of Surfactant-A-protein and which still retains the ability to bind TLR4 and inhibit TLR4 signaling pathway(s). In certain embodiments, the isolated peptide may comprise an amino acid sequence that is at least 80% identical to a portion of SEQ ID NO:1 or 80% identical to any of SEQ ID NOS:3-7. In other embodiments, the isolated peptide may comprise an amino acid sequence that is at least 90% identical to a portion of SEQ ID NO:1 or 90% identical to any of SEQ ID NOS:3-7. In yet other embodiments, the isolated peptide may comprise an amino acid sequence that has 1-5 amino acid changes when compared to a portion of SEQ ID NO:1 or any of SEQ ID NOS:3-7; for example, the isolated peptide may comprise an amino acid sequence that differs from any of SEQ ID NOS:3-7 by 5 amino acids or less, by 4 amino acids or less, by 3 amino acids or less, by two amino acids or less, or by one amino acid or less.

In certain embodiments of the presently disclosed inventive concepts, the composition may include multiple isolated peptides as described herein above.

In certain embodiments, the presently disclosed inventive concepts further include a method of producing any of the compositions described herein above. Said method may comprise any of the steps described herein or otherwise known in the art. The compositions of the presently disclosed inventive concepts may be prepared according to methods known in the art, particularly in light of the disclosure and examples set forth herein. The starting materials used to synthesize the compositions of the presently disclosed inventive concepts are commercially available or capable of preparation using methods known in the art.

In certain embodiments, the presently disclosed inventive concepts also include an isolated nucleic acid segment encoding any of the compositions described herein above. In addition, a recombinant vector comprising said nucleic acid segment, as well as a recombinant host cell comprising said recombinant vector, are also contemplated within the scope of the presently disclosed inventive concepts. In certain embodiments, the recombinant host cell produces the peptide composition.

TABLE 1

| SEQ | Amino Acid |
|---|---|
| ID NO: | Sequence |
| 8 | GDFRY |
| 9 | DFRYS |
| 10 | FRYSD |
| 11 | RYSDG |
| 12 | YSDGT |
| 13 | SDGTP |
| 14 | DGTPV |
| 15 | GTPVN |
| 16 | TPVNY |
| 17 | PVNYT |
| 18 | VNYTN |
| 19 | NYTNW |
| 20 | YTNWY |
| 21 | TNWYR |
| 22 | NWYRG |
| 23 | WYRGE |
| 24 | GDFRYS |
| 25 | DFRYSD |
| 26 | FRYSDG |
| 27 | RYSDGT |
| 28 | YSDGTP |
| 29 | SDGTPV |
| 30 | DGTPVN |
| 31 | GTPVNY |
| 32 | TPVNYT |
| 33 | PVNYTN |
| 34 | VNYTNW |
| 35 | NYTNWY |
| 36 | YTNWYR |
| 37 | TNWYRG |
| 38 | NWYRGE |
| 39 | GDFRYSD |
| 40 | DFRYSDG |
| 41 | FRYSDGT |
| 42 | RYSDGTP |
| 43 | YSDGTPV |
| 44 | SDGTPVN |
| 45 | DGTPVNY |
| 46 | GTPVNYT |
| 47 | TPVNYTN |
| 48 | PVNYTNW |
| 49 | VNYTNWY |
| 50 | NYTNWYR |
| 51 | YTNWYRG |
| 52 | TNWYRGE |
| 53 | GDFRYSDG |
| 54 | DFRYSDGT |
| 55 | FRYSDGTP |
| 56 | RYSDGTPV |
| 57 | YSDGTPVN |
| 58 | SDGTPVNY |
| 59 | DGTPVNYT |
| 60 | GTPVNYTN |
| 61 | TPVNYTNW |
| 62 | PVNYTNWY |
| 63 | VNYTNWYR |
| 64 | NYTNWYRG |
| 65 | YTNWYRGE |
| 66 | GDFRYSDGT |
| 67 | DFRYSDGTP |
| 68 | FRYSDGTPV |
| 69 | RYSDGTPVN |
| 70 | YSDGTPVNY |
| 71 | SDGTPVNYT |
| 72 | DGTPVNYTN |
| 73 | GTPVNYTNW |
| 74 | TPVNYTNWY |
| 75 | PVNYTNWYR |
| 76 | VNYTNWYRG |
| 77 | NYTNWYRGE |
| 78 | GDFRYSDGTP |
| 79 | DFRYSDGTPV |
| 80 | FRYSDGTPVN |
| 81 | RYSDGTPVNY |
| 82 | YSDGTPVNYT |
| 83 | SDGTPVNYTN |
| 84 | DGTPVNYTNW |
| 85 | GTPVNYTNWY |
| 86 | TPVNYTNWYR |
| 87 | PVNYTNWYRG |
| 88 | VNYTNWYRGE |
| 89 | GDFRYSDGTPV |
| 90 | DFRYSDGTPVN |
| 91 | FRYSDGTPVNY |
| 92 | RYSDGTPVNYT |
| 93 | YSDGTPVNYTN |
| 94 | SDGTPVNYTNW |
| 95 | DGTPVNYTNWY |
| 96 | GTPVNYTNWYR |
| 97 | TPVNYTNWYRG |
| 98 | PVNYTNWYRGE |
| 99 | GDFRYSDGTPVN |
| 100 | DFRYSDGTPVNY |
| 101 | FRYSDGTPVNYT |
| 102 | RYSDGTPVNYTN |
| 103 | YSDGTPVNYTNW |
| 104 | SDGTPVNYTNWY |
| 105 | DGTPVNYTNWYR |
| 106 | GTPVNYTNWYRG |
| 107 | TPVNYTNWYRGE |
| 108 | GDFRYSDGTPVNY |
| 109 | DFRYSDGTPVNYT |
| 110 | FRYSDGTPVNYTN |
| 111 | RYSDGTPVNYTNW |
| 112 | YSDGTPVNYTNWY |
| 113 | SDGTPVNYTNWYR |
| 114 | DGTPVNYTNWYRG |
| 115 | GTPVNYTNWYRGE |
| 116 | GDFRYSDGTPVNYT |
| 117 | DFRYSDGTPVNYTN |
| 118 | FRYSDGTPVNYTNW |
| 119 | RYSDGTPVNYTNWY |
| 120 | YSDGTPVNYTNWYR |
| 121 | SDGTPVNYTNWYRG |
| 122 | DGTPVNYTNWYRGE |
| 123 | GDFRYSDGTPVNYTN |
| 124 | DFRYSDGTPVNYTNW |
| 125 | FRYSDGTPVNYTNWY |
| 126 | RYSDGTPVNYTNWYR |
| 127 | YSDGTPVNYTNWYRG |
| 128 | SDGTPVNYTNWYRGE |
| 129 | GDFRYSDGTPVNYTNW |
| 130 | DFRYSDGTPVNYTNWY |
| 131 | FRYSDGTPVNYTNWYR |
| 132 | RYSDGTPVNYTNWYRG |
| 133 | YSDGTPVNYTNWYRGE |
| 134 | GDFRYSDGTPVNYTNWY |
| 135 | DFRYSDGTPVNYTNWYR |
| 136 | FRYSDGTPVNYTNWYRG |
| 137 | RYSDGTPVNYTNWYRGE |
| 138 | GDFRYSDGTPVNYTNWYR |
| 139 | DFRYSDGTPVNYTNWYRG |
| 140 | FRYSDGTPVNYTNWYRGE |
| 141 | GDFRYSDGTPVNYTNWYRG |
| 142 | DFRYSDGTPVNYTNWYRGE |
| 143 | YVGLT |
| 144 | VGLTE |
| 145 | GLTEG |
| 146 | LTEGP |
| 147 | TEGPS |
| 148 | EGPSP |
| 149 | GPSPG |
| 150 | PSPGD |
| 151 | SPGDF |
| 152 | PGDFR |
| 153 | YVGLTE |
| 154 | VGLTEG |
| 155 | GLTEGP |
| 156 | LTEGPS |
| 157 | TEGPSP |
| 158 | EGPSPG |
| 159 | GPSPGD |
| 160 | PSPGDF |
| 161 | SPGDFR |
| 162 | PGDFRY |
| 163 | YVGLTEG |
| 164 | VGLTEGP |
| 165 | GLTEGPS |
| 166 | LTEGPSP |
| 167 | TEGPSPG |
| 168 | EGPSPGD |
| 169 | GPSPGDF |
| 170 | PSPGDFR |
| 171 | SPGDFRY |
| 172 | PGDFRYS |
| 173 | YVGLTEGP |
| 174 | VGLTEGPS |
| 175 | GLTEGPSP |
| 176 | LTEGPSPG |
| 177 | TEGPSPGD |
| 178 | EGPSPGDF |
| 179 | GPSPGDFR |
| 180 | PSPGDFRY |
| 181 | SPGDFRYS |
| 182 | PGDFRYSD |
| 183 | YVGLTEGPS |
| 184 | VGLTEGPSP |
| 185 | GLTEGPSPG |
| 186 | LTEGPSPGD |
| 187 | TEGPSPGDF |
| 188 | EGPSPGDFR |
| 189 | GPSPGDFRY |
| 190 | PSPGDFRYS |
| 191 | SPGDFRYSD |
| 192 | PGDFRYSDG |
| 193 | YVGLTEGPSPG |
| 194 | VGLTEGPSPGD |
| 195 | GLTEGPSPGDF |
| 196 | LTEGPSPGDFR |
| 197 | TEGPSPGDFRY |
| 198 | EGPSPGDFRYS |
| 199 | GPSPGDFRYSD |
| 200 | PSPGDFRYSDG |
| 201 | SPGDFRYSDGT |
| 202 | PGDFRYSDGTP |
| 203 | YVGLTEGPSPGD |
| 204 | VGLTEGPSPGDF |
| 205 | GLTEGPSPGDFR |
| 206 | LTEGPSPGDFRY |
| 207 | TEGPSPGDFRYS |
| 208 | EGPSPGDFRYSD |
| 209 | GPSPGDFRYSDG |
| 210 | PSPGDFRYSDGT |
| 211 | SPGDFRYSDGTP |
| 212 | YVGLTEGPSPGDF |
| 213 | VGLTEGPSPGDFR |
| 214 | GLTEGPSPGDFRY |
| 215 | LTEGPSPGDFRYS |
| 216 | TEGPSPGDFRYSD |
| 217 | EGPSPGDFRYSDG |
| 218 | GPSPGDFRYSDGT |
| 219 | PSPGDFRYSDGTP |
| 220 | YVGLTEGPSPGDFR |
| 221 | VGLTEGPSPGDFRY |
| 222 | GLTEGPSPGDFRYS |
| 223 | LTEGPSPGDFRYSD |
| 224 | TEGPSPGDFRYSDG |
| 225 | EGPSPGDFRYSDGT |
| 226 | GPSPGDFRYSDGTP |
| 227 | YVGLTEGPSPGDFRY |
| 228 | VGLTEGPSPGDFRYS |
| 229 | GLTEGPSPGDFRYSD |
| 230 | LTEGPSPGDFRYSDG |
| 231 | TEGPSPGDFRYSDGT |
| 232 | EGPSPGDFRYSDGTP |
| 233 | YVGLTEGPSPGDFRYS |
| 234 | VGLTEGPSPGDFRYSD |
| 235 | GLTEGPSPGDFRYSDG |
| 236 | LTEGPSPGDFRYSDGT |
| 237 | TEGPSPGDFRYSDGTP |
| 238 | YVGLTEGPSPGDFRYSD |
| 239 | VGLTEGPSPGDFRYSDG |
| 240 | GLTEGPSPGDFRYSDGT |
| 241 | LTEGPSPGDFRYSDGTP |
| 242 | YVGLTEGPSPGDFRYSDG |
| 243 | VGLTEGPSPGDFRYSDGT |
| 244 | GLTEGPSPGDFRYSDGTP |
| 245 | YVGLTEGPSPGDFRYSDGT |
| 246 | VGLTEGPSPGDFRYSDGTP |

In certain embodiments, the presently disclosed inventive concepts are further directed to a pharmaceutical composition comprising any of the isolated peptide compositions described herein above or otherwise contemplated herein, in combination with a pharmaceutically acceptable carrier (or biologically-active molecule or vaccine-adjuvant). In addition, a pharmaceutical composition comprising a nucleic acid segment encoding said peptide composition in combination with a pharmaceutically acceptable carrier is also contemplated in accordance with the presently disclosed inventive concepts.

In certain embodiments, the presently disclosed inventive concepts are also directed to a method of using any of the pharmaceutical compositions described herein above. Said method includes administering an effective amount of the pharmaceutical composition to a patient in need thereof.

In certain embodiments, the presently disclosed inventive concepts are also directed to a method of inhibiting TLR4 signaling. Said method comprises contacting a cell expressing TLR4 on a surface thereof with any of the isolated peptide compositions described herein above or otherwise contemplated herein, wherein the peptide composition binds to TLR4 on the surface of the cell and inhibits TLR4 signaling by the cell.

In certain embodiments, the presently disclosed inventive concepts are also directed to a method of inhibiting at least one inflammatory parameter, such as but not limited to, TNF-α, myeloperoxidase, NF-κB, IL-β, and pro-IL-1β. The method comprises contacting a cell with any of the isolated peptide compositions described herein above or otherwise contemplated herein and/or administering any of the pharmaceutical compositions described herein above or otherwise contemplated herein to a patient in which it is desired to inhibit the at least one inflammatory parameter.

In certain embodiments, the presently disclosed inventive concepts are further directed to a method of decreasing the occurrence and/or severity of inflammation associated with a disease condition. The method comprises administering an effective amount of a composition (or pharmaceutical composition) as described in detail herein above to a subject suffering from or predisposed to the inflammation/disease condition, thereby decreasing the occurrence and/or severity of inflammation associated with the disease condition in the subject. In one embodiment, TLR4 signaling is involved in the inflammation/disease condition.

Any inflammatory conditions known in the art or otherwise contemplated herein may be treated in accordance with the presently disclosed inventive concepts. Non-limiting examples of disease conditions having inflammation associated therewith include infection-related or non-infectious inflammatory conditions in the lung (i.e., sepsis, lung infections, Respiratory Distress Syndrome (RDS), bronchopulmonary dysplasia, etc.); asthma, chronic obstructive pulmonary disease (COPD), cystic fibrosis, chronic conditions, pneumonia, acute respiratory distress syndrome (ARDS), bronchopulmonary dysplasia (BPD), and infant respiratory distress syndrome (IRDS); viral, bacterial, and fungal infections; infectious diseases (local and systemic infections) at other mucosal sites; osteoarthritis; GI-associated infection-related or non-infectious inflammatory conditions, as well as infection-related or non-infectious inflammatory conditions in other organs (i.e., colitis, Inflammatory Bowel Disease, irritable bowel syndrome, ulcerative colitis, Crohn's disease, diabetic nephropathy, hemorrhagic shock, eye inflammation, skin inflammation, psoriasis, genitourinary inflammation, etc.); inflammation-induced cancer (i.e., cancer progression in colon, lung, breast cancer, as well as cancer progression in patients with colitis or Inflammatory Bowel Disease); autoimmune diseases (i.e., rheumatoid arthritis, etc.); and the like.

In certain embodiments, the presently disclosed inventive concepts yet further include a method of decreasing the occurrence and/or severity of infection in a patient. The method comprises administering to the patient a therapeutically effective amount of any of the pharmaceutical compositions described herein above or otherwise contemplated herein. The pharmaceutical composition may further include at least one additional agent, wherein the agent acts in concert or synergistically with the isolated peptide(s) of the pharmaceutical composition.

In one embodiment, the additional agent(s) may be an anti-infective agent. Non-limiting examples of anti-infective agents that may be utilized in accordance with the presently disclosed inventive concepts include aminoglycosides (i.e., amikacin, gentamicin, kanamycin, neomycin, netilmicin, tobramycin, paromomycin, spectinomycin, etc.); carbapenems (i.e., ertapenem, doripenem, imipenem/cilastatin, meropenem, etc.); cephalosporins (cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftazidime, ceftibuten, ceftizoxime, ceftriaxone, cefepime, ceftaroline fosamil, ceftobiprole, etc.); glycopeptides; lincosamides; lipopeptides; macrolides (i.e., azithromycin, clarithromycin, dirithromycin, erythromycin, roxithromycin, troleandomycin, telithromycin, spiramycin, etc.); monobactams (aztreonam, etc.); nitrofurans (i.e., furazolidone, nitrofurantoin, etc.); oxazolidonones; polypeptides (i.e., bacitracin, colistin, polymyxin B, etc.); quinolones (i.e., ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, etc.); penicillins (i.e., amoxicillin, ampicillin, carbenicillin, cloxacillin, dicloxacillin, flucloxacillin, mezlocillin, methicillin, nafcillin, oxacillin, penicillin G or V, piperacillin, temocillin, and ticarcillin, etc.); penicillins combined with beta-lactamase inhibitors (i.e., piperacillin/tazobactam, amoxicillin/clavulanate, ampicillin/sulbactam, ticarcillin/clavulanate, etc.); sulfonamides; tetracyclines (i.e., demeclocycline, doxycycline, minocycline, oxytetracycline, tetracycline, etc.); trimethoprim; sulfamethoxazole and trimethoprim (Bactrim); other antibiotics known in the art or otherwise contemplated herein, and combinations and derivatives thereof.

In another embodiment, the additional agent(s) may be an anti-inflammatory agent. Non-limiting examples of anti-inflammatory agents that may be utilized in accordance with the presently disclosed inventive concepts include monoclonal antibodies (such as but not limited to, anti-TNF and/or anti-IL-1β monoclonal antibodies) and receptor antagonists approved for use in colitis and rheumatoid arthritis.

In another embodiment, the additional agent(s) may be a surfactant. Non-limiting examples of surfactants that may be utilized in accordance with the presently disclosed inventive concepts include CUROSURF® (Chiesi Farmaceutici S.p.A. Corp., Parma, Italy), ALVEOFACT® (Lyoma Mark Pharma GmbH, Germany), NEOSURF™ (Discovery Laboratories, Inc., Warrington, Pa.), Surfact, INFASURF® (ONY Inc., Amerherst, N.Y.), SURVANTA® (Abbott Laboratories Corp., Abbott Park, Ill.), SURFAXIN® (Discovery Laboratories, Inc., Warrington, Pa.), EXOSURF™ (Smithkline Beecham Corp., Philadelphia, Pa.), VENTICUTE® (NYCOMED GmbH Corp, Germany), KL₄ (Discovery Laboratories, Inc., Warrington, Pa.), other surfactants known in the art and/or contemplated herein, and combinations and derivatives thereof.

While the inventive concepts are not limited to a specific mechanism of decreasing the occurrence and/or severity of infection in a patient, the compositions disclosed herein may act by enhancing TLR4-induced bacterial uptake and intracellular lysis of bacteria.

In certain embodiments, the presently disclosed inventive concepts are further directed to methods of decreasing the occurrence and/or severity of endotoxic shock in a patient. The method comprises administering to the patient a therapeutically effective amount of any of the pharmaceutical compositions described herein above or otherwise contemplated herein. The pharmaceutical composition may further include at least one additional anti-infective agent, wherein the anti-infective agent acts in concert or synergistically with the isolated peptide(s) of the pharmaceutical composition.

In certain embodiments, the presently disclosed inventive concepts are further directed to methods of decreasing the occurrence and/or severity of at least one symptom of endotoxic shock in a patient. The method comprises administering to the patient a therapeutically effective amount of any of the pharmaceutical compositions described herein above or otherwise contemplated herein. The pharmaceutical composition may further include at least one additional anti-infective agent, wherein the anti-infective agent acts in concert or synergistically with the isolated peptide(s) of the pharmaceutical composition.

In certain embodiments, the presently disclosed inventive concepts further include a method of promoting lung development and/or function in infants born pre-term (who are unable to make enough surfactant). Said method comprises administering an effective amount of a composition (or pharmaceutical composition) as described in detail herein above to a subject to promote lung development and/or function and/or maintain immune homeostasis. The composition (or pharmaceutical composition) may be administered alone or in combination with surfactant (i.e., currently available lipid-based clinical surfactants). Non-limiting examples of surfactants that may be utilized in accordance with the presently disclosed inventive concepts include CUROSURF® (Chiesi Farmaceutici S.p.A. Corp., Parma, Italy), ALVEOFACT® (Lyoma Mark Pharma GmbH, Germany), NEOSURF™ (Discovery Laboratories, Inc., Warrington, Pa.), Surfact, INFASURF® (ONY Inc., Amerherst, N.Y.), SURVANTA® (Abbott Laboratories Corp., Abbott Park, Ill.), SURFAXIN® (Discovery Laboratories, Inc., Warrington, Pa.), EXOSURF™ (Smithkline Beecham Corp., Philadelphia, Pa.), VENTICUTE® (NYCOMED GmbH Corp, Germany), KL₄ (Discovery Laboratories, Inc., Warrington, Pa.), other surfactants known in the art and/or contemplated herein, and combinations and derivatives thereof.

In certain embodiments, the presently disclosed inventive concepts are further directed to an adjuvant composition, such as a vaccine adjuvant composition, that includes any of the isolated peptide compositions described herein above or otherwise contemplated herein. The presently disclosed inventive concepts further include a method that includes administering the adjuvant composition to a patient in combination with a second agent (such as but not limited to, a vaccine), whereby the administration of the adjuvant composition maintains and/or enhances an immune response raised against the second agent.

In certain embodiments, the presently disclosed inventive concepts also include a method of inhibiting metastatic properties of a cancer cell. The method comprises contacting a cancer cell with any of the isolated peptide compositions described herein above or otherwise contemplated herein and/or administering any of the pharmaceutical compositions described herein above or otherwise contemplated herein to a patient suffering from or predisposed to cancer. In particular embodiments, the metastatic properties of the cancer cell that are inhibited are LPS-TLR4-stimulated metastatic properties of cancer cells. The method may further comprise the administration of at least one additional agent, such as but not limited to, a chemotherapeutic agent, wherein the additional agent acts in concert or synergistically with the isolated peptide composition.

In another embodiment, the additional agent(s) may be an anti-inflammatory agent. Non-limiting examples of anti-inflammatory agents that may be utilized in accordance with the presently disclosed inventive concepts include monoclonal antibodies (such as but not limited to, anti-TNF and/or anti-IL-1β monoclonal antibodies) and receptor antagonists approved for use in colitis and rheumatoid arthritis.

In another embodiment, the additional agent(s) may be a surfactant. Non-limiting examples of surfactants that may be utilized in accordance with the presently disclosed inventive concepts include CUROSURF® (Chiesi Farmaceutici S.p.A. Corp., Parma, Italy), ALVEOFACT® (Lyoma Mark Pharma GmbH, Germany), NEOSURF™ (Discovery Laboratories, Inc., Warrington, Pa.), Surfact, INFASURF® (ONY Inc., Amerherst, N.Y.), SURVANTA® (Abbott Laboratories Corp., Abbott Park, Ill.), SURFAXIN® (Discovery Laboratories, Inc., Warrington, Pa.), EXOSURF™ (Smithkline Beecham Corp., Philadelphia, Pa.), VENTICUTE® (NYCOMED GmbH Corp, Germany), KL₄ (Discovery Laboratories, Inc., Warrington, Pa.), other surfactants known in the art and/or contemplated herein, and combinations and derivatives thereof.

In certain embodiments, the presently disclosed inventive concepts further include a method of decreasing the occurrence and/or severity of metastasis (and/or slowing the rate of metastasis) in a patient suffering from or predisposed to cancer. The method comprises administering to the patient a therapeutically effective amount of any of the pharmaceutical compositions described herein above or otherwise contemplated herein. The method may further comprise the administration of at least one additional agent, such as but not limited to, a chemotherapeutic agent that acts in concert or synergistically with the isolated peptide composition.

In one embodiment, the additional agent(s) may be an anti-inflammatory agent. Non-limiting examples of anti-inflammatory agents that may be utilized in accordance with the presently disclosed inventive concepts include monoclonal antibodies (such as but not limited to, anti-TNF and/or anti-IL-1β monoclonal antibodies) and receptor antagonists approved for use in colitis and rheumatoid arthritis.

In another embodiment, the additional agent(s) may be a surfactant. Non-limiting examples of surfactants that may be utilized in accordance with the presently disclosed inventive concepts include CUROSURF® (Chiesi Farmaceutici S.p.A. Corp., Parma, Italy), ALVEOFACT® (Lyoma Mark Pharma GmbH, Germany), NEOSURF™ (Discovery Laboratories, Inc., Warrington, Pa.), Surfact, INFASURF® (ONY Inc., Amerherst, N.Y.), SURVANTA® (Abbott Laboratories Corp., Abbott Park, Ill.), SURFAXIN® (Discovery Laboratories, Inc., Warrington, Pa.), EXOSURF™ (Smithkline Beecham Corp., Philadelphia, Pa.), VENTICUTE® (NYCOMED GmbH Corp, Germany), KL₄ (Discovery Laboratories, Inc., Warrington, Pa.), other surfactants known in the art and/or contemplated herein, and combinations and derivatives thereof.

In certain embodiments, the presently disclosed inventive concepts further include a method of decreasing the occurrence and/or severity of inflammation-induced carcinogenesis in a patient suffering from or predisposed to cancer. The method comprises administering to the patient a therapeutically effective amount of any of the pharmaceutical compositions described herein above or otherwise contemplated herein. The method may further comprise the administration of at least one additional agent (as described herein above) that acts in concert or synergistically with the isolated peptide composition.

In certain embodiments, the presently disclosed inventive concepts also include kits that include any of the peptide compositions disclosed or otherwise contemplated herein. For example but not by way of limitation, kits that include one or more of the peptide compositions may be utilized for in vivo administration thereof to a mammalian patient. The kit may also include instructions for administering the composition to the mammalian patient. In addition, the kit may optionally also contain one or more other compositions for use in accordance with the methods described herein. For example, but not by way of limitation, the kit may include a pharmaceutical composition that comprises one or more of the peptide compositions disposed in a pharmaceutically acceptable carrier (including, but not limited to, a PEGylated peptide or other form of a peptide conjugate as disclosed herein). Alternatively, the kit may include an additional agent such as but not limited to, a chemotherapeutic agent, an anti-inflammatory agent, a surfactant, and/or an anti-infective agent.

### EXAMPLES

Examples are provided hereinbelow. However, the presently disclosed inventive concepts are to be understood to not be limited in its application to the specific experimentation, results and laboratory procedures. Rather, the Examples are simply provided as one of various embodiments and are meant to be exemplary, not exhaustive.

### Example 1

### Surfactant Protein-A and Toll-like Receptor 4 Modulate Immune Functions of Lung Dendritic Precursor Cells Harvested from Preterm Baboons

Preterm infants are highly susceptible to infections; this increased susceptibility to infections is associated with perturbed development and extreme immaturity of the immune system. Antigen-presenting cells have an important role in pathogen-uptake and processing as well as in regulating inflammatory and adaptive host immune responses. Among various types of antigen-presenting cells, dendritic cells (DCs) have been recognized as the most potent. In the past, several studies have confirmed an immunomodulatory role of lung DCs against a variety of antigens in adult humans and animal models with a mature immune system; however, until recently, the phenotypes and functions of lung DCs remained poorly known in preterm babies. The inventor was the first to isolate a unique low-density lung cell population from preterm baby baboons (Awasthi et al. (2009) Immunol Cell Biol. 87:419-427). The results of this study showed that the cells have a density similar to adult baboon lung DCs, are lineage-negative and defective in responding to infectious stimuli. Overall these results suggest that despite having similar isolation characteristics, this unique cell-population harvested from fetal lung does not belong to conventional immature or mature DC categories. Based on these unique properties, they were identified as DC-precursor cells. Recent results from the inventor further demonstrate that the fetal DC-precursor cells express low level of DC-markers, and incubation with DC-promoting cytokines (GM-CSF, IL-4 and TNF-α) induces differentiation of these fetal cells into typical DCs (unpublished results).

The DCs are well known to coordinate innate and adaptive immunity via pathogen-pattern recognition receptors, such as Toll-like receptors (TLR), mannose receptors, scavenger receptors and collections (such as but not limited to, surfactant protein-A (SPA)). Deficiencies or functional defects of pathogen-pattern recognition receptors can negatively affect the DC functions, compromise the host defense and lead to serious consequences in early life-periods of preterm babies. The inventor's previous studies have mainly focused on SPA, a major part of lung surfactant that lines the alveoli, and TLR4, a potent membrane-receptor that senses both pathogen-associated and damage-associated molecular patterns (PAMP and DAMPs).

The inventor's previous studies observed that the expression of SPA and TLR4 is undetectable or negligible in lung tissues of fetuses (at 67%-75% of complete gestation term) under steady-state conditions, and increases to the levels equivalent to adult counterparts as the gestation period reaches closer to term. However, preterm birth, mechanical injury (ventilation-associated) and infection significantly influence lung-homeostasis and decrease the alveolar SPA pools to significantly low levels. In contrast, the expression of TLR4 is increased in preterm babies with lung infections.

To this end, recent understanding in the field suggests that SPA and TLR4 both enhance phagocytosis. The lack of SPA in alveoli may compromise the uptake of pathogens. However, an exaggerated activation of TLR4 can lead to chronic inflammatory response or “cytokine storm.” This pattern correlates well with fulminating infection (low SPA=low phagocytic uptake) and inflammatory response (increased TLR4=increased amounts of pro-inflammatory cytokines) in preterm babies having lung infection. These results led the inventor to hypothesize that the introduction of SPA-based clinical surfactants and TLR4-antagonists may compensate for the loss of SPA and downregulate an exaggerated TLR4-mediated inflammatory response, respectively. It has also been learned recently that SPA interacts with TLR4 in vitro and in lung. Thus, introduction of SPA may have an effect on TLR4-mediated immune responses. In this Example, the immunomodulatory effects of native SPA and recombinant TLR4-MD2 proteins were investigated on selected immune functions (phagocytosis and cytokine response) of fetal baboon lung DC-precursor cells and compared with those of adult baboon lung DCs. The results presented in this Example demonstrate that in both adult and fetal systems, pulsing of cells with SPA and TLR4-MD2 proteins increases the phagocytic uptake of Escherichia coli bioparticles. When added together, no additive effect was demonstrated on phagocytic function of DCs. Co-incubation of cells with SPA and TLR4-MD2 proteins, however, significantly inhibits the TLR4-MD2-induced release of TNF-α against E. coli.

Overall, the results presented in this Example support a significant role of SPA in improving innate phagocytic function and in suppressing the TLR4-mediated deleterious inflammatory response against infectious stimuli.

Materials and Methods for Example 1:

Baboon lung tissues: The animal studies were approved by Institutional Animal Care and Use Committees, Environmental Health and Safety or Institutional Biosafety Committee of the University of Oklahoma Health Science Center, Oklahoma City, Okla. (OUHSC). Baboon (P. anubis) colonies were maintained at Baboon Resources, OUHSC, Oklahoma City, Okla. At the time of necropsy, whole fresh lung or a lobe of lung from fetal (delivered at 125 days of gestation (125 dGA); complete term is 185 dGA) and adult baboons (age range 10-22 years) was collected in RPMI 1640 medium containing 2 mM glutamine, 1 mM N-2-Hydroxyethylpiperazine-N′-2-ethanesulfonic acid (HEPES), 10 μg/ml gentamicin, 100 U/ml penicillin, 100 μg/ml streptomycin and 10% fetal bovine serum (low endotoxin <10EU/ml, FBS; Invitrogen, Carlsbad, Calif.). None of the animals recruited in this study showed any clinical sign of infection or lung pathology. Gross and microscopic examinations of all major viscera and the placenta revealed no signs of inflammation or infection.

Purification of baboon lung SPA: SPA was purified from bronchoalveolar lavage fluid of a normal healthy adult baboon by a slight modification of the procedure described previously (Yang et al. (2005) J. Biol Chem., 280:34447-34457). The bronchoalveolar lavage fluid was collected from an adult baboon lung by instilling endotoxin-free, sterile normal saline (endotoxin-free 0.9% NaCl, 1.9-2 L with approximately 90% recovery). The lavage fluid was centrifuged, and the supernatant was concentrated using a tangential flow filtration technique (10 kDa hollow fiber filter; GE Healthcare Bio-Sciences Corp, NJ). The surfactant lipids were removed using isobutyl alcohol (1:5 ratio lavage:isobutyl alcohol). The delipidated protein was centrifuged at 5,000×g for 15 minutes at room temperature, dried under nitrogen gas, and subsequently completely dried in a lyophilizer (Labconco, MO). The dried lavage residue was rehydrated in extraction buffer (25 mM Tris (pH 7.5), 0.15 M NaCl, and 20 mM octyl-β-D-glucoside) overnight at 4° C. Rehydrated surfactant was extracted six times with extraction buffer by vortex mixing and centrifugation at 20,000×g for 30 minutes at 4° C. Insoluble SPA was then suspended in solubilization buffer (5 mM HEPES (pH 7.5), 0.02% sodium azide) and dialyzed for 72 hours against four changes of the solubilization buffer. Insoluble protein was removed by centrifugation at 50,000×g for 30 minutes at 4° C., and supernatant was adjusted to 20 mM CaCl₂ and 1 M NaCl to re-precipitate SPA. Precipitated SPA was pelleted by centrifugation at 50,000×g for 30 minutes at 4° C., and washed two times in 5 mM HEPES (pH 7.5), 20 mM CaCl₂ and 1 M NaCl. The SPA was suspended in 5 mM HEPES, 5 mM EDTA (pH 7.5) and dialyzed for 72 hours against four changes of the solubilization buffer to remove EDTA. The purified SPA was dialyzed against four changes of the endotoxin-free, highly-purified water (Invitrogen, CA) for 72 hours to remove any remaining EDTA or salts (CaCl₂ and NaCl). Finally, purified SPA was lyophilized completely and resuspended in endotoxin-free Dulbecco's phosphate buffered saline. The purified protein was filter-sterilized using a 0.2 μm low-protein binding, HT Tuffryn membrane filter (Pall Life Sciences, NY) and stored frozen at −80° C. The protein concentration of purified SPA was measured by microBCA method (Pierce, IL).

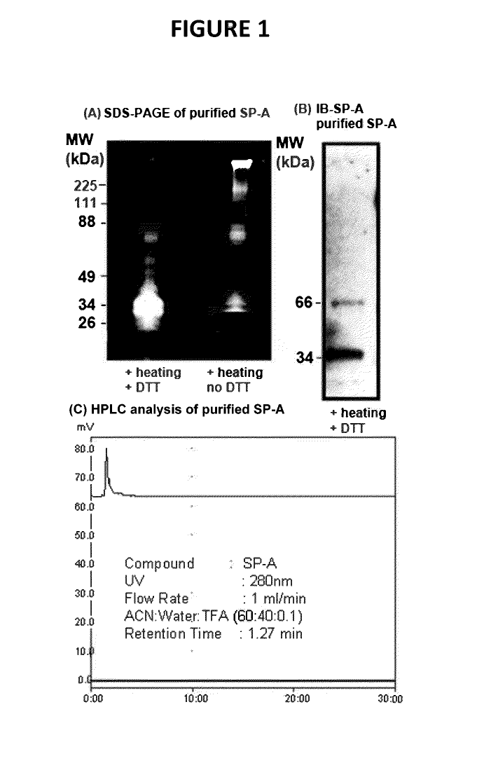

All the purification steps were performed under aseptic conditions using endotoxin-free solutions and reagents. The endotoxin concentration was measured using the End-point chromogenic Limulus Amebocyte Lysate (LAL) assay (Charles River Laboratories, MA). The purity of the SPA protein was confirmed by SDS-PAGE and Western blotting using the procedures described earlier (Awasthi et al. (1999) Am J Respir Crit Care Med, 160:942-949; and Awasthi et al. (2001) Am J Respir Crit Care Med, 163:389-397). The isolated protein was further characterized by high performance liquid chromatography (HPLC) on a Phenomenex C-18 reverse phase column using solvents acetonitrile/water/trifluoroacetic acid (60:40:1) at 1 ml/min, with the UV detector set at 280 nm. The retention time of SPA was determined to be 1.3 minutes (FIG. 1).

Culture of KG-1-derived DCs and isolation of primary lung DCs: KG-1 cells (Bone marrow myeloblast cells derived from a leukemia patient; ATCC, VA) were cultured in the presence of recombinant human-GM-CSF (100 ng/ml), IL-4 (100 ng/ml) and TNF-α (40 ng/ml) (all the cytokines were purchased from PeproTech, NJ) for a period of 5 days (Ackerman et al. (2003) J Immunol. 170:4178-88; Bharadwaj et al., (2005) J Surg Res. 127:29-38; and Hulette et al., (2001) Arch Dermatol Res. 293:147-58). The phenotype and morphology of the KG-1-derived DCs were confirmed by flow cytometry and light microscopy, respectively (Awasthi and Cooper, 2006). The KG-1-derived DCs were included as model system to optimize the amounts of effector molecules (purified baboon lung SPA and recombinant TLR4-MD2 proteins).

Isolation of Adult Baboon Lung DC or Fetal Baboon Lung DC-Precursor Population:

Freshly collected lobe of the lung or whole lung samples of adult and fetal baboons were transported on ice in RPMI 1640 medium containing 2 mM glutamine, 1 mM HEPES, 10 μg/ml gentamicin, 100 U/ml penicillin, 100 μg/ml streptomycin and 10% FBS. After a mild mechanical disruption, the single cell suspension was seeded in a tissue culture flask (Nalge-Nunc International Corp, NY) at a density of 30-50×10⁶ leukocytes/175 cm² flask in RPMI 1640 medium containing 2 mM glutamine, 1 mM HEPES, 10 μg/ml gentamicin, 100 U/ml penicillin, 100 μg/ml streptomycin and 10% FBS. The light-density DC-populations were harvested using OptiPrep cell-separation solution (density 1.32 g/ml, Accurate Chemicals, NY) (Awasthi and Cooper (2006) Cell Immunol 240:31-40; and Awasthi et al. (2009) Immunol Cell Biol. 87:419-27). The immunophenotype and basic characteristics of the lung DC-population isolated from fetal and adult baboons have been described earlier (Awasthi et al., 2009). Here, first the immunophenotype of KG-1-derived DCs and fetal baboon lung DC-precursors or adult baboon lung DCs were compared by flow cytometry. Briefly, cells were suspended in Dulbecco's phosphate buffered saline (DPBS) containing 1% FBS and 0.09% sodium azide and incubated with fluorochrome-conjugated antibodies to HLA-DP, DQ, DR, CD11c, CD40, CD80, CD86 (typical DC-markers) as described earlier.

Expression of endogenous TLR4 by flow-cytometry and western blotting: The harvested cells were suspended in 100 μl DPBS containing 1% FBS and 0.09% sodium azide. Previously titrated phycoerythrinin (PE)-conjugated anti-human TLR4 antibody (BD Biosciences, CA), was added at the ratio of 1 μg antibody per 1×10⁶ cells. Cells and antibody were incubated for 30 minutes on ice in the dark. The cells were washed three times with PBS containing 1% FBS and 0.09% sodium azide and fixed in freshly prepared 0.5% paraformaldehyde. The cells were run through an automated dual laser excited FACS Calibur at the Flow and Imaging Core Facility (OUHSC, Oklahoma City). The histogram and dot-plot charts were obtained and analyzed using Summit V4.3 software (Dako Colorado Inc, CO). The isotype control antibody-stained cells served as controls for background staining.

For western immunoblotting, whole cell lysates were prepared in lysis buffer (50 mM Tris-HCl, pH 7.4) containing 1% Igepal, 0.25% sodium deoxycholate, 150 mM NaCl, 1 mM EDTA, 1 mM phenyl methyl sulfonyl fluoride (PMSF), and 1 μg/ml each of leupeptin and pepstatin. After protein estimation, about 15 μg of total proteins were fractionated by Novex 4-20% Tris-glycine gradient SDS-PAGE gel (Invitrogen, CA). Separated proteins were electro-transferred onto a nitrocellulose membrane using an iBlot gel transfer device (Invitrogen, CA). The non-specific sites were blocked by incubating the membrane with 7% skim milk in Tris-buffered saline with 0.4% Tween-20 (TBST). The blocked membrane was incubated overnight at 4° C. with monoclonal antibody against TLR4 (clone HTAl25; ebioscience, CA) diluted 1:1000 in TBST. The membrane was washed and incubated with horseradish peroxidase (HRP)-conjugated-anti-rabbit-IgG antibody (Sigma-Aldrich, MO) diluted 1:1000 in TBST. The immunoreactive bands were detected by SuperSignal West Femto detection reagent (Thermo Fischer Scientific, IL).

Cellular distribution of exogenously added TLR4-MD2 protein: Since KG-1-derived-DCs, adult baboon lung-DCs and fetal baboon lung DC-precursor cells expressed negligible amounts of TLR4 protein; the cells were pulsed with recombinant human TLR4-MD2 proteins (RnD Systems, MN). The cellular distribution of exogenously-added recombinant TLR4-MD2 protein was investigated by confocal microscopy and flow cytometry.

Labeling of recombinant TLR4-MD2 protein with ALEXA FLUOR® 594 fluorescent dye (Molecular Probes, Inc., Eugene, Oreg.). Recombinant TLR4-MD2 protein was labeled with ALEXA FLUOR® 594 fluorescent dye using a microscale protein labeling kit (Molecular Probes, Inc., Eugene, Oreg.) optimized for labeling proteins with molecular weights between 12 and 150 kDa, as per the manufacturer's directions. Briefly, 40 μg of recombinant TLR4-MD2 protein (at the concentration of 1 mg/ml in DPBS) was labeled with ALEXA FLUOR® 594 carboxylic acid, succinimidyl ester (Excitation/Emission wavelengths: 590/617 nm). The pH of the protein was adjusted to 8.3 using 1/10 volume of 1M sodium bicarbonate, and ALEXA FLUOR® 594 reactive dye stock solution (12.2 nmol/μl) was added to the protein. The protein:dye mixture was incubated for 15 minutes at room temperature. Fluorochrome-conjugated protein was then separated from unconjugated dye using a spin filter conditioned with gel resin. The spin filter loaded with reaction mixture was centrifuged at 16,000×g for 1 minute. The absorbance of the ALEXA FLUOR® 594 dye-conjugated TLR4-MD2 protein was read at 280 nm and 590 nm using UV/Vis NanoDrop ND-1000 spectrophotometer (NanoDrop Technologies, DE) and degree of labeling (DOL) was determined using the following formula:

$$Protein concentration ( mg \text{/} mL ) = \frac{[ A 280 - 0.56 ( A 590 ) ] \times dilution factor}{A 280 of protein at 1 mg \text{/} mL}$$

$$Protein concentration ( M ) = \frac{Protein concentration ( mg \text{/} mL )}{Protein molecular weight ( Da )}$$

$$DOL = ( moles dye ) / ( mole protein ) = \frac{A 590 \times dilution factor}{90 , 0 \; 00 \times protein concentration ( M )}$$

Where A280 and A590 are the protein's absorbance at 280 nm and at 590 nm, respectively, the value of 0.56 is a correction factor for the fluorophore's contribution to the A280, and 90,000 cm⁻¹ M⁻¹ is the approximate molar extinction coefficient of the ALEXA FLUOR® 594 dye.

Cellular distribution of ALEXA FLUOR® 594-conjugated TLR4-MD2 protein by confocal microscopy. The KG-1-derived DCs were seeded at a density of 2.5×10⁵ cells per well in an 8-well chamber slide (Nalge Nunc international, NY) in serum-free Opti-MEM medium (Invitrogen, CA). The cells were then incubated with 10 μg of ALEXA FLUOR® 594-conjugated recombinant TLR4-MD2 protein (DOL ^{˜}4.0) for 1 hour and 4 hours at 37° C. in 5% CO₂ atmosphere. Thirty minutes prior to the completion of incubation period, the VYBRANT® DiO cell labeling solution (5 nM final concentration, Molecular Probes, Inc., Eugene, Oreg.) and Hoechst 33342 dye (0.3 μg/ml final concentration, Invitrogen-Life Technologies, Grand Island, N.Y.) were added to the cells for staining cell-cytoplasm and nucleus, respectively. Finally, the cells were washed twice in Opti-MEM medium, fixed using VECTASHIELD®-antifade mounting medium (Vector Laboratories, Inc., Burlingame, Calif.) and observed under Leica TCS SP2 AOBS (Acousto Optical Beam Splitter) multi-photon laser confocal microscope at the Flow and Image Cytometry laboratory (OUHSC, Oklahoma City). Images were acquired with 63× objective lens (at excitation/emission wavelengths: 405/410-550 nm for Hoechst 33342 dye, 488/500-550 nm for DiO dye, and 594/610-660 nm for ALEXA FLUOR® 594 dye) and were analyzed using the Leica TCS software (Leica Microsystems CMS, Mannheim, Germany). Finally, the images acquired were merged and composite pictures were obtained.

Localization of ALEXA FLUOR® 594-conjugated TLR4-MD2 protein by flow cytometry. The KG-1-derived DCs were suspended in Opti-MEM medium at the density of 2.5×10⁵ cells per 100 μl and incubated with 10 μg ALEXA FLUOR® 594-conjugated recombinant TLR4-MD2 protein. After 1 hour and 4 hours of incubation, cells were washed three times in fresh Opti-MEM medium, re-suspended in 500 μl of 37° C. pre-warmed DPBS, and run on Influx Cell Sorter (BD Biosciences, CA) at the Flow and Image Cytometry laboratory (OUHSC, Oklahoma City). The cells were gated to remove debris and histogram charts were obtained at 624-40 nm emission wavelengths. Cell-staining with ALEXA FLUOR® 594-conjugated TLR4-MD2 protein was analyzed using Summit V4.3 software (Dako Colorado Inc, CO).

Phagocytosis assay: In this Example, pHrodo-conjugated, heat-killed E. coli K12 (encapsulated) bioparticles (Invitrogen-Molecular Probes, CA) were employed. The pHrodo-fluorescent label offers an advantage over other conventional methods, in that it fluoresces only in acidic conditions (i.e., after the bioparticles are taken inside the intracellular lysosomes) (Invitrogen-Molecular Probes, CA). To ensure that the fluorescence relates to phagocytosed pHrodo-conjugated E. coli bioparticles only, the phagocytosis reaction mix was imaged by confocal microscopy. Briefly, the KG-1-derived DCs were seeded at a density of 2.5×10⁵ cells/well in an 8-well chamber slide and incubated with pHrodo-conjugated E. coli K12 bioparticles (one cell-to-^{˜}300 bacterial bioparticles) for 3 hours. The Hoechst 33342 dye was added to the cells (0.3 μg/ml final concentration, Invitrogen-Molecular Probes, CA). The cells were washed once, re-suspended in Opti-MEM medium, fixed with Vectashield-antifade mounting medium (Vector Laboratories, CA), and observed under Leica TCS SP2 AOBS (Acousto Optical Beam Splitter) multi-photon laser confocal microscope (at excitation/emission wavelengths: 550/600 nm for pHrodo-labeled bioparticles, 405/410-550 nm for Hoechst 33342 dye) and under brightfield. Images taken at different wavelengths were merged, and composite pictures were obtained.

After confirming that the fluorescence is of phagocytosed bioparticles, comprehensive experiments were performed in presence and absence of effector molecules (SPA and TLR4-MD2 proteins). The cells were pulsed with purified, native baboon lung SPA and recombinant human TLR4-MD2 or MD2 proteins (RnD Systems, MN) for an hour prior to phagocytosis assay with pHrodo-conjugated, heat-killed E. coli K12 bioparticles (Invitrogen-Molecular Probes, CA). MD2 protein was also included, because SPA is known to interact with MD2, and it was questioned if MD2 can influence the immune functions of DC-population. The assay was performed in serum-free Opti-MEM medium (Invitrogen, CA), as described by the manufacturer (Invitrogen-Molecular Probes, CA). The assay mixtures were incubated for another 3 hours at 37° C. in 5% CO₂ incubator. The fluorescence readings were taken at 550 nm excitation and 600 nm emission wavelengths using SpectraMax M2 spectrofluorometer (Molecular Devices, CA).

The phagocytosis index was calculated by subtracting the average fluorescence intensity of the reaction with bioparticles alone from the control (basal without any effector molecules) and experimental wells. Finally, the percent effect was calculated using the following formula:

% effect=(Net experimental phagocytosis/Net basal phagocytosis)×100%

The percent phagocytosis of E. coli bioparticles was also confirmed by fluorescence microscopy in representative reaction wells. The cell-free supernatants were collected after taking the fluoremetric readings and stored at −80° C. for further analysis.

Cytokine (TNF-α) measurement: The TNF-α levels were measured in cell-free supernatants by enzyme linked immunosorbent assay (ELISA) using a commercially available kit (eBioscience, CA), as per the manufacturer's instructions. Briefly, the microwells of a 96 well plate were coated with diluted purified anti-human TNF-α antibody. The wells were washed, and nonspecific sites were blocked. Diluted recombinant human TNF-α (7.8-500 pg/ml standard) and cell-free-supernatant (1:10) were added to the antibody-coated wells, and the plate was incubated overnight at 4° C. The next day, the plate was washed and incubated with biotin-conjugated anti-human TNF-α antibody followed by avidin-horseradish peroxidase and substrate solution. The reaction was stopped by adding 2 NH₂SO₄ and read at 450 nm (Molecular Devices, CA).

Statistical Analysis: The results were analyzed by Student's t-test for statistical significance using Prism software (Graphpad, San Diego, Calif.). p<0.05 was considered significant.

Results of Example 1:

The fetal baboon lung DC-population collected from the top of the density gradient were unique to fetal baboons, and were not identified in adult baboons. The morphologic features and phenotypic characteristics have been described in the inventor's publication (Awasthi et al., 2009, the contents of which are expressly incorporated herein by reference).

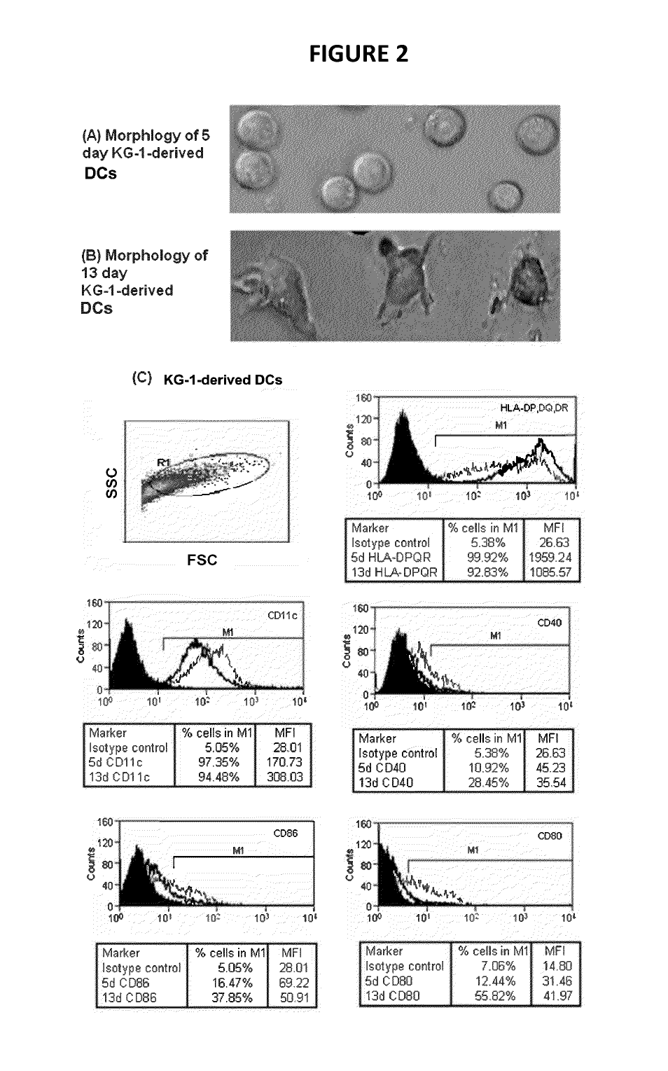

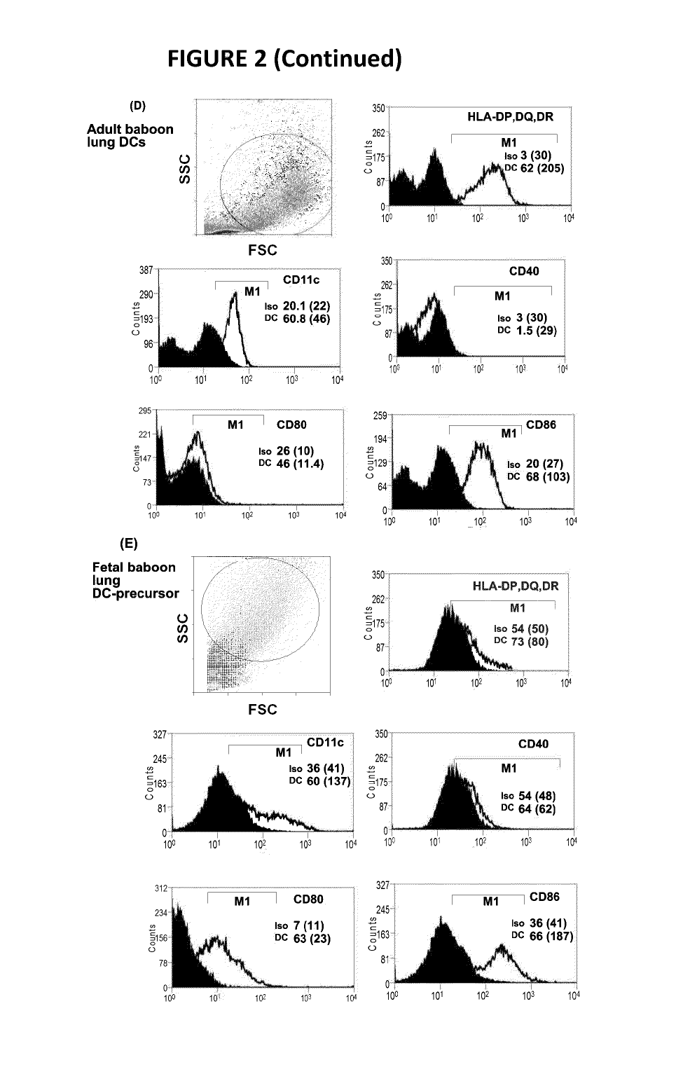

Prior to conducting experiments with adult baboon lung DCs or fetal baboon lung DC-precursor cells, the KG-1-DCs (harvested on 6th day of culture) were used in the initial experiments to optimize the concentration of effector molecules. Morphologically, KG-1-derived DCs harvested on day 5 of culture were round and did not show any tentacles or dendrites (typical of immature DCs). However, after 13 days, the DCs developed dendrites and an irregular shape, characteristic features of mature DCs (FIGS. 2A and B). Flow cytometry analysis of cell-surface-antibody-stained cells showed that KG-1 cells transform into DCs after 13 days and express HLA-DP,DQ,DR, CD11c, CD40, CD80 and CD86 cell-surface markers, characteristics of typical DCs (FIG. 2C). On comparison, it was found that the KG-1-derived DCs express DC-markers to levels similar to those in adult baboon lung DCs (FIGS. 2C and 2D). The fetal baboon lung DC-precursor cells showed negligible levels of DC-markers except CD80 and CD86 (FIG. 2E). Later, primary lung DCs isolated from healthy adult baboons and DC-precursor cells isolated from fetal baboons were used to study the immunomodulatory effects of SPA and TLR4-MD2 proteins against infectious stimuli.

Characterization of purified native baboon lung SPA. To elucidate the effects of SPA, first native SPA protein was first purified from bronchoalveolar lavage fluid specimens of a normal, healthy adult baboon (Awasthi et al. (1999) Am J Respir Crit Care Med 160:942-949; and Awasthi et al. (2001) Am J Respir Crit Care Med 163:389-397). The purity and identity of the native baboon lung SPA was confirmed by SDS-PAGE, western blotting and HPLC. Under partially-reducing conditions (heating and no DTT), SPA separated as an oligomer, ^{˜}100 kDa trimer, and a 66 kDa dimer on the SDS-PAGE gel. Under reducing condition (heating+DTT), SPA ran as a 32-34 kDa monomer and 66 kDa partially reduced dimer (FIG. 1A). The purified baboon lung SPA protein was also immunoblotted with SPA antibody which identified the SPA-specific protein bands (FIG. 1B). The HPLC chromatogram further confirmed the purity of SPA (FIG. 1C). Since the TLR4-MD2 proteins are less abundant in the biological system and it is difficult to obtain native TLR4-MD2 protein in sufficient quantity, the recombinant human-TLR4-MD2 protein (RnD Systems, MN) was used. As an adaptor molecule to TLR4, recombinant MD2 protein was also included in the phagocytosis assays.

Since TLR4 is a potent receptor for endotoxin, the presence of endotoxin can significantly influence the results. All of the solutions and reagents were prepared in endotoxin-free water, and all assays were performed in an aseptic environment. Endotoxin concentration was measured in the purified baboon SPA, and in the reconstituted TLR4-MD2 and MD2 proteins by the chromogenic LAL method (Charles River Lab, MA). The endotoxin concentration was negligible in purified baboon SPA (0.0003 ng/μg protein) and in recombinant TLR4-MD2 and MD2 protein suspensions (≦0.006 ng/μg protein).

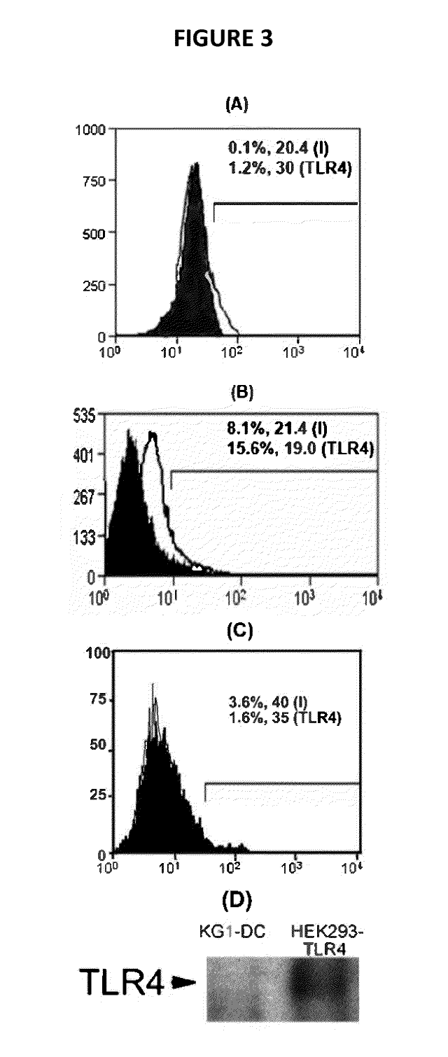

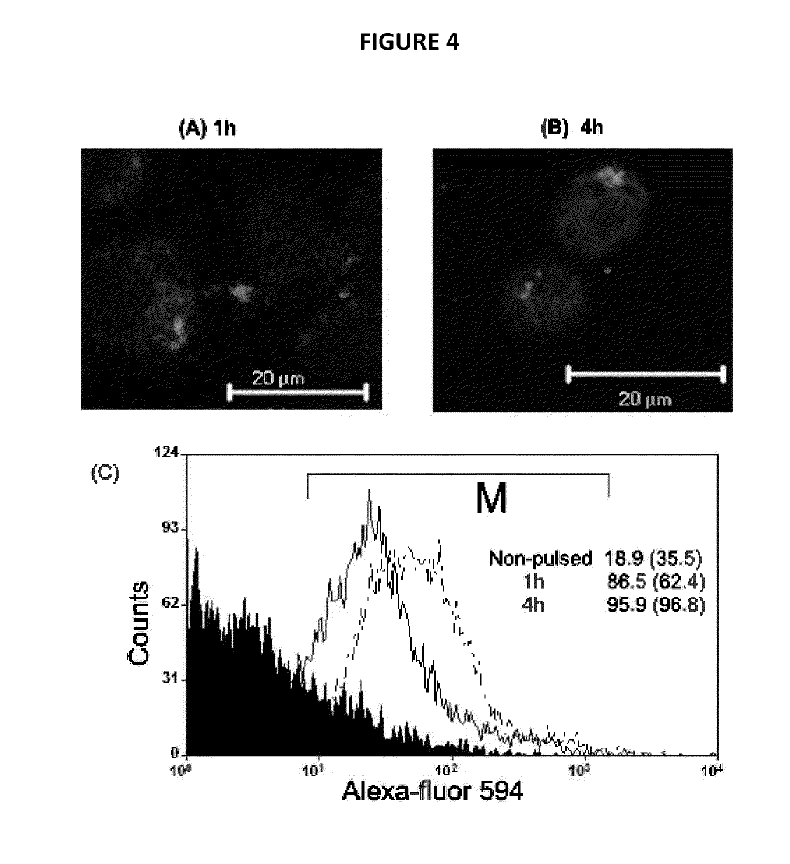

KG-1-derived DCs and primary DCs express negligible TLR4 protein under normal conditions; exogenously added TLR4-MD2 protein localizes mainly on the cell surface. The basal cell-surface expression of TLR4 on primary adult baboon lung DCs and KG-1 derived DCs was negligible (FIG. 3). The expression of TLR4 was also undetectable in fetal lung DC-precursor cells (FIG. 3). Thus, DCs were pulsed with recombinant TLR4-MD2 protein prior to the phagocytosis assay. The localization of ALEXA FLUOR® 594-conjugated TLR4-MD2 protein in the KG-1-derived DCs was tracked and studied by confocal microscopy. Confocal images showed that the TLR4-MD2 protein localized mainly on the cell membrane (FIGS. 4A and 4B). Flow-cytometric analysis of DCs pulsed with ALEXA FLUOR® 594-labeled TLR4-MD2 protein showed that there is an increase in MFI values and percent number of cells staining positive for ALEXA FLUOR® 594 stain (Molecular Probes, Inc., Eugene, Oreg.). These findings further confirmed that the TLR4-MD2 protein localizes on the cell-surface (FIG. 4C).

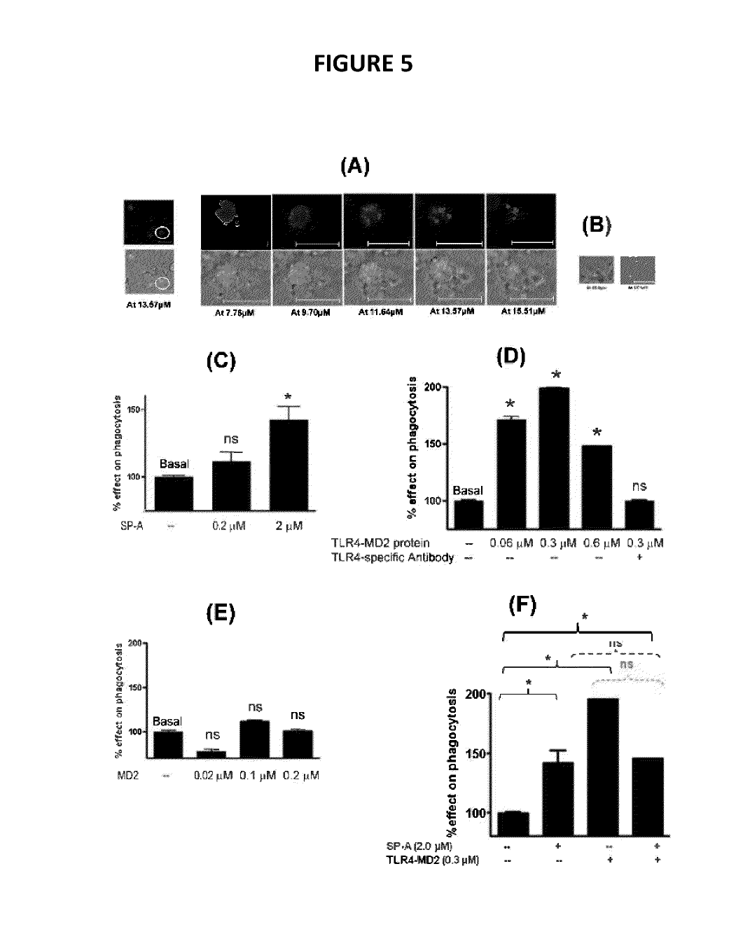

Exogenous addition of native SPA and recombinant TLR4-MD2 proteins increases the phagocytic uptake of E. coli bioparticles. In this Example, pHrodo-labeled, heat-killed encapsulated E. coli K12 bioparticles were utilized for investigating the phagocytic ability of DCs. First, the fluorescence of phagocytosed bioparticles in KG-1-derived DCs was confirmed by confocal microscopy. The images showed that only the phagocytosed bioparticles fluoresce (FIG. 5A). In FIG. 5A, a fluorescent cell is focused that has taken up the bioparticles. In contrast, the extracellular bioparticles in the same field, settled at the bottom of the well (z-stack slice #69.8 μm) or floating towards the top (z-stack slice #1.94 μm) do not emit any fluorescence at all (FIGS. 5A and 5B).

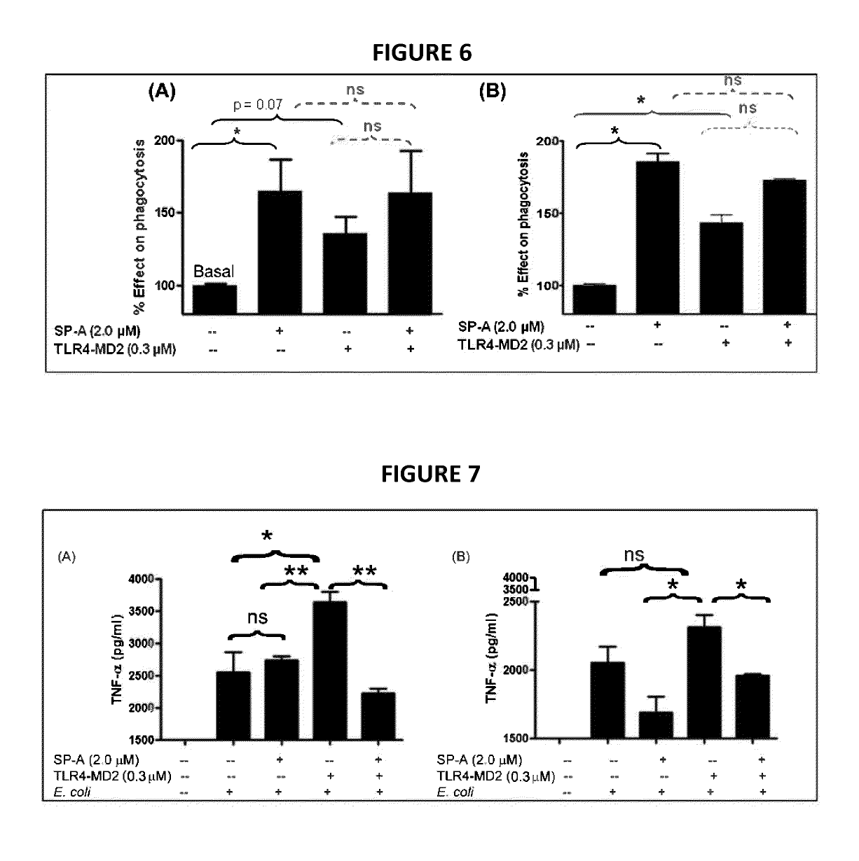

In comprehensive phagocytosis experiments, the fluorescence signal reflecting the red fluorescence emitted by phagocytosed pHrodo-labeled E. coli bioparticles, was measured by spectrofluorometry using identical wavelengths setting. Briefly, the KG-1-derived DCs were incubated with purified baboon lung SPA protein±TLR4-MD2 protein. The % net effect on phagocytosis was calculated in the presence of effector molecules (TLR4-MD2, MD2 and SPA) after normalizing with the basal phagocytosis in the absence of the effector molecules. The percent phagocytosis calculated by fluorescence microscopy (number of fluorescing cells/total number of cells in a composite of 5 different fields) correlated with the phagocytosis indices calculated by the spectrofluorometry methods. These data demonstrate that both SPA and TLR4-MD2 proteins increase the phagocytic uptake of E. coli bioparticles by 1.5-2 fold in concentration-dependent manner (p<0.05, FIGS. 5C and 5D). The MD2 protein alone did not affect the phagocytosis (FIG. 5E). Next, the phagocytosis assay was performed with primary lung DC or DC-precursor population in presence of purified SPA (2 μM) and TLR4-MD2 (0.3 μM) proteins at concentrations that provided maximum phagocytic uptake in KG1-derived DCs (FIGS. 5F and 6). The results demonstrate that, similar to KG-1-derived DCs, the phagocytic uptake of E. coli is increased in the presence of exogenous SPA (p<0.05) and TLR4-MD2 protein in primary baboon lung DCs. When SPA and TLR4-MD2 proteins were added together, the phagocytic uptake of E. coli remained increased as compared to basal level; however, no additive effect was observed (FIG. 6).

SPA reduces the TLR4-MD2 protein-induced TNF-α release against E. coli. TNF-α levels were measured in cell-free supernatants of primary adult baboon lung DCs and fetal baboon lung DC-precursor cells treated with SPA±TLR4-MD2 proteins after 3 hours of phagocytosis reaction. Addition of purified lung SPA did not induce the secretion of TNF-α by DCs in response to E. coli, but pulsing with TLR4-MD2 protein increased the TNF-α secretion significantly (p<0.05). However, when the SPA and TLR4-MD2 proteins were added together to the cells and incubated further for another 3 hours with E. coli, the TNF-α levels were equivalent to those incubated without any exogenous protein or with SPA only (FIG. 7). There was no major difference in responses elicited by DC-populations harvested from adult or fetal baboon lung (FIGS. 7A and 7B), except that the amounts of TNF-α were lower in fetal cells.

Discussion for Example 1

The inventor's results on fetal baboon bone marrow-derived DCs as well as the reports of others with monocytes provided evidence that DC functions (i.e., phagocytosis and cytokine secretion) are impaired during prenatal and neonatal periods. However, recent understanding indicates that the tissue-resident DCs are different than the circulating or bone marrow-derived DCs. Results obtained by the inventor also demonstrate that fetal baboon lung cells are at precursor stage, express negligible levels of DC-markers, and are functionally defective in responding to infectious stimuli. Although the developmental stage of the fetal lung DC-population remains to be completely elucidated in fetal baboons, they have been identified by the inventor as DC-precursor cells because they convert into typical DCs after incubation with DC-promoting cytokines (unpublished data).

One possibility is that since these cells are not fully equipped with TLR or other pathogen-pattern recognition receptors because of developmental immaturity, the DC-precursor cells are not capable of capturing the microorganisms. SPA also serves as a pathogen-pattern recognition receptor and is known to stimulate DC-maturation and phagocytic uptake of infectious organisms. However, at 125 days of gestation, SPA is not detectable. NICU care and proper clinical management induce expression of both SPA and TLR4 which reaches to optimal levels under normal conditions. However, despite an advanced and sophisticated clinical care, preterm babies are more prone to infection, and infection and ventilator-associated lung injury remarkably perturb the expression of SPA and TLR4. Specifically, lavage pools of SPA are decreased, and tissue expression of TLR4 is increased. These published results suggest that introduction of SPA may help maintain the tissue homeostasis and exert anti-infective and anti-inflammatory effects. The present Example was designed to determine if the introduction of SPA will impact the functions of DCs in the lungs of preterm babies.

The present example was focused on studying selected immune functions: phagocytosis and cytokine secretion against infectious stimuli. Primary cells were pulsed with purified or recombinant protein preparations for two reasons: first, the genetic-transfection of primary DCs will require longer time for efficient protein expression, and longer incubation may induce phenotypic changes in DCs; and second, the protein-pulsing mimics the physiological scenario, because both SPA and TLR4 proteins are known to exist in soluble extracellular, cell surface as well as in intracellular forms under steady-state conditions. MD2 was also included in conjunction with TLR4, because it serves as an important adaptor molecule to TLR4 and binds to SPA. However, MD2 does not carry an intracellular signaling TIR domain and does not affect the phagocytic function of DCs on its own (FIG. 4). A few investigations have shown that SPA and TLR4 proteins interact in vitro. Although the functional relevance of this interaction in fetal or neonatal lungs remains largely unexplored, the results of the present Example demonstrate that SPA reduces TLR4-MD2-induced cytokine release against infectious stimuli.

The present Example demonstrates that an exogenous addition of SPA and TLR4-MD2 proteins in the DC population increases phagocytic uptake of encapsulated E. coli (FIGS. 5 and 6). These findings are of clinical importance because encapsulated bacteria resist phagocytosis by antigen-presenting cells and mount an aggressive inflammatory response. It is possible that purified SPA can also directly kill some of the Gram-negative bacteria by increasing the membrane permeability as reported earlier. The SPA-induced phagocytosis of E. coli bioparticles by DC-precursor cells point towards the importance of SPA in improving immune functions in preterm babies. Interestingly, SPA suppresses the TLR4-mediated cytokine release significantly in response to infectious stimuli (FIG. 7). Similar results have been obtained in Ureaplasma infection models in an established macrophage cell line RAW 264.7 and in mice. The present Example further supports the role of SPA in improving the innate immune functions in preterm babies.

The results of this Example are of clinical importance because surfactant preparations currently-used in NICUs do not contain SPA. Ultimately, this Example supports the idea of reformulating the currently-available clinical surfactant preparations to contain SPA and their clinical usage in NICU.

### Example 2

### A Novel TLR4-interacting Surfactant Protein-A-Derived Peptide Suppresses LPS-Induced TLR4 Expression and TNF-α Release

Published reports suggest that the bronchoalveolar lavage pools (extracellular pools) of SPA are significantly reduced in lungs of infected patients and animal models. In contrast, the TLR4 expression is increased. The reduction in the amounts of SPA, and the simultaneous increase in TLR4 expression corroborates well with the clinical condition of patients having fulminant infection and inflammation, respectively. In these clinical scenarios, the introduction of SPA should facilitate clearance of pathogens and attenuate inflammation. However, currently-available clinical surfactants do not contain SPA or SP-D. Thus, a great need has been felt for designing a shorter fragment of SPA as well as reformulating the surfactant.

Interestingly, recently published reports suggested that SPA directly binds to TLR4. The in vivo evidence of such an interaction has been lacking, and its functional relevance has not been fully elucidated. In this Example, the binding of SPA to TLR4-MD2 in non-infected, normal baboon lung tissues was determined by co-immunoprecipitation/immunoblotting, and in vitro by a microwell-based method. Next, a bioinformatics approach was used to examine the interaction between SPA and TLR4-MD2 proteins. In conjunction, potential binding regions were identified in an in silico model of the SPA-TLR4-MD2 complex. Based on the information obtained from the bioinformatics analysis, an SPA-derived peptide library was synthesized. Studies were further extended to investigate the functional relevance of SPA-TLR4 interaction in a dendritic cell system. The present Example demonstrates that similar to native SPA, an SPA-derived peptide (SPA4; SEQ ID NO:3) binds to TLR4-MD2 protein, inhibits expression of TLR4 and reduces the release of TNF-α in response to the most potent TLR4-ligand: Gram-negative bacteria-derived lipopolysaccharide (LPS).

Materials and Methods of Example 2:

Animals: The animal studies were approved by the Institutional Animal Care and Use and Institutional Biosafety Committees at the University of Oklahoma Health Science Center (OUHSC), Oklahoma City, Okla. Baboons (Papio anubis) were maintained at the Baboon Research Resource, OUHSC, Oklahoma City, Okla. At the time of necropsy, lung tissue or bronchoalveolar lavage fluid specimens were obtained from normal healthy adult baboons. Gross and microscopic examinations of major viscera as well as the lung tissue specimens from these baboons showed no signs of inflammation or infection.

Preparation of baboon lung tissue homogenate: The frozen lung tissue samples were homogenized in a buffer containing 1% Igepal CA630, 0.1% sodium dodecyl sulfate, and protease inhibitors (1 μM leupeptin, 1 mM ethylenediamine tetraacetic acid, 0.7 mg/L pepstatin and 0.2 mM phenylmethyl sulphonyl fluoride; Sigma-Aldrich, MO) at a concentration of 100 mg tissue/ml buffer (Awasthi et al., 1999; Awasthi et al., 2001). The tissue homogenates were centrifuged to remove cell debris, and total protein concentration was measured in supernatants using the MicroBCA protein estimation kit (Pierce, IL).

Western blotting: The inventor has recognized the cross-reactivity of anti-human-SPA- and anti-human-TLR4-antibodies with respective antigens in baboons, and studied the expression of SPA and TLR4 in lung tissue homogenates of fetal and adult baboons, and neonate baboons having Bronchopulmonary dysplasia (Awasthi et al., 1999; Awasthi et al., (2008) Dev Comp Immunol 32:1088-1098). Here, using western blotting, the immunoreactivity of these antibodies with respective antigens was first confirmed in baboon lung tissue homogenates to ensure the integrity of the antigens. Lysates of HEK-293 cells stably-transfected with human-TLR4-cDNA (provided by Invivogen, CA), and purified human- and baboon-lung SPA proteins served as positive controls.

The protein samples were prepared in SDS-PAGE sample buffer without dithiothreitol (DTT)+no heating (non-reducing), without DTT+heating at 100° C. for 5 minutes (partially-reducing) or with DTT+heating at 100° C. for 5 minutes (reducing). The samples were loaded and separated on a SDS-PAGE gel (8% running and 5% stacking gel or Novex 4-20% Tris-glycine gel, Invitrogen, CA). Separated proteins were then electro-transferred overnight onto a nitrocellulose membrane. The nonspecific sites were blocked by incubating the membrane in 7% skim milk diluted in Tris-buffered saline containing 0.4% Tween 20 (TBST). The membranes were then incubated with anti-human-SPA polyclonal antibody (Awasthi et al., 1999; Awasthi et al., 2001) or TLR4 antibody (eBioscience, CA) (Awasthi et al., 2008), diluted 1:1000 in TBST, for 1 hour at room temperature. The membrane was washed and then incubated with horseradish peroxidase (HRP)-conjugated-anti-mouse or anti-rabbit IgG antibody (1:1000 diluted in TBST; Sigma-Aldrich, MO). The immunoreactive bands were detected by Supersignal West Pico or Femto chemiluminescent substrate (Pierce, IL).

Immunoprecipitation of lung-SPA or TLR4 and cross-immunoblotting: After confirming the reactivity of the antibodies and the integrity of TLR4 and SPA proteins in baboon lung tissue homogenates, the physical binding between the two proteins was examined by immunoprecipitation/cross-immunoblotting. The SPA and TLR4 proteins were immunoprecipitated from baboon lung tissue homogenates and cross-immunoblotted with anti-human-TLR4 and SPA antibodies, respectively. The SPA (IP-SPA) and TLR4 (IP-TLR4) were immunoprecipitated using Primary Seize Immunoprecipitation kit (Pierce, IL) as per the manufacturer's instructions. Approximately 200 μg of anti-human-TLR4 or SPA antibody (Awasthi et al., 2001; Awasthi et al., 2008) was conjugated to the AminoLink plus coupling gel column at 4° C. overnight. Five hundred μg to 1 mg of total lung tissue homogenate protein was loaded into the columns and the immunoprecipitation reaction was performed overnight at 4° C. The IP-SPA and IP-TLR4 were eluted from the antibody-bound column using ImmunoPure elution buffer. No Calcium was added to the immunoprecipitation reaction at any step. Also, none of the buffers in the kit contained calcium.

Various amounts of IP-TLR4 and IP-SPA were run on SDS-PAGE gels. The separated proteins were then transferred on nitrocellulose membrane using the i-Blot system (Invitrogen, CA). For cross-immunoblotting, IP-SPA and IP-TLR4 were immunoblotted with anti-TLR4 and anti-SPA antibodies, respectively, as described above. Positive controls included lung tissue homogenate protein, purified human SPA and lysate-protein of HEK-293 cells-transfected with full-length, human-TLR4-cDNA. Negative controls included IP-SPA and IP-TLR4 immunoblotted with a non-specific antibody, and immunoprecipitates from columns where the lung tissue homogenate or the primary antibody had been omitted.

Purification and characterization of native Baboon SPA: SPA was purified from bronchoalveolar lavage fluid of an adult baboon by a modification of the procedure described previously (Yang et al., 2005). The bronchoalveolar lavage fluid was collected from an adult baboon lung by instilling endotoxin-free, sterile normal saline (endotoxin-free 0.9% NaCl, 1.9-2 L with approximately 90% recovery). The lavage fluid was centrifuged, and the supernatant was concentrated using a tangential flow filtration technique (10 kDa hollow fiber filter; GE Healthcare Bio-Sciences Corp, NJ). The surfactant lipids were removed using isobutyl alcohol (1:5 ratio lavage:isobutyl alcohol). The delipidated protein was centrifuged at 5000×g for 15 minutes at room temperature, dried under nitrogen gas, and subsequently completely dried in a lyophilizer (Labconco, MO). The dried lavage residue was rehydrated in extraction buffer (25 mM Tris, pH 7.5, 0.15 M NaCl, and 20 mM octyl-β-D-glucoside) overnight at 4° C. Rehydrated surfactant was extracted six times with extraction buffer by vortex mixing and centrifugation at 20,000×g for 30 minutes at 4° C. Insoluble SPA was then suspended in solubilization buffer (5 mM HEPES, pH 7.5, 0.02% sodium azide) and dialyzed for 72 hours against four changes of the solubilization buffer. Insoluble protein was removed by centrifugation at 50,000×g for 30 minutes at 4° C., and supernatant was adjusted to 20 mM CaCl₂ and 1 M NaCl to re-precipitate SPA. Precipitated SPA was pelleted by centrifugation at 50,000×g for 30 minutes at 4° C., and washed two times in 5 mM HEPES pH 7.5, 20 mM CaCl₂ and 1 M NaCl. The SPA was suspended in 5 mM HEPES, 5 mM EDTA, pH 7.5 and dialyzed for 72 hours against four changes of the solubilization buffer to remove EDTA. The purified SPA was dialyzed against four changes of endotoxin-free, highly-purified water (Invitrogen, CA) for 72 hours to remove any remaining EDTA or salts (CaCl₂ and NaCl). Finally, purified SPA was lyophilized completely and resuspended in endotoxin-free Dulbecco's phosphate buffered saline. The purified protein was filter-sterilized using a 0.2 μm low-protein binding, HT Tuffryn membrane filter (Pall Life Sciences, NY) and stored frozen at −80° C. The protein concentration of purified SPA was measured by microBCA method (Pierce, IL).

All the purification steps were performed under aseptic conditions using endotoxin-free solutions and reagents. The endotoxin concentration was measured using the End-point chromogenic Limulus Amebocyte Lysate (LAL) assay (Charles River Laboratories, MA). The purity of the SPA protein was confirmed by SDS-PAGE and Western blotting using the procedures described above.

Interaction between purified baboon lung SPA, SPA-peptides and TLR4-MD2 proteins using a microwell-based method: The binding between the purified baboon lung SPA, SPA-peptides and recombinant TLR4-MD2 and MD2 proteins was studied in vitro using a microwell-based method (Awasthi et al. (2004) Respir Res 5:28). The soluble, recombinant TLR4-MD2 protein (R&D Systems, MN) consisted of a mixture of recombinant human-TLR4 and MD2 proteins. The recombinant extracellular domain of human TLR4 protein (Glu 24-Lys 631 amino acids), was joined with a DNA sequence encoding the signal peptide from human CD33 and a 10× histidine tag at the C-terminus (Accession #O00206). For MD2 protein, a DNA sequence encoding the signal peptide from human CD33 was joined with the mature region of human MD-2 (mature region, Glu 17-Asn 160 amino acids) and a 10× histidine tag at the C-terminus (Accession #Q9Y6Y9). The chimeric proteins were expressed in a mouse myeloma cell line, NS0 (R&D Systems, MN). The proteins were obtained from the manufacturer in carrier-free condition and reconstituted in PBS containing 0.1% low-endotoxin BSA (MP Biomedicals, OH). MD2, an adaptor molecule for TLR4, is expressed by immune cells, and is known to bind TLR4 in a non-covalent manner. Thus, the binding of SPA to the recombinant MD2 protein (R&D Systems, MN) was also studied.

For the binding assay, microwell ultra-high-protein binding Immunolon 4 HBX strips (Thermo Scientific, MA) were coated with soluble recombinant-TLR4-MD2 protein or MD2 protein (R&D Systems, 250 ng per well, diluted in 0.1 M NaHCO₃, pH 9.6) overnight at room temperature. The plates were washed three times, and non-specific sites were blocked for 2 hours at room temperature using phosphate buffered saline containing 0.1% triton-X 100 and 3% nonfat milk (Buffer A). The wells were washed and incubated for 3 hours at 37° C. with purified baboon lung SPA (0.125-10 μg), SPA4 peptide (2-20 μg; SEQ ID NO:3) or adult baboon lung tissue homogenate protein (10-100 μg) diluted in 20 mM Tris (pH 7.4) buffer containing 0.15 M NaCl, 5 mM CaCl₂ or equal amount of bovine serum albumin (BSA) protein. The wells were washed with Buffer A and incubated with anti-human SPA antibody (1:1000 diluted in Buffer A) for 2 hours at room temperature followed by HRP-conjugated secondary antibody. The immune-complex was detected using 3,3′,5,5′-tetramethylbenzidine (TMB) substrate system (Sigma-Aldrich, MO). The reaction was stopped with 2 NH₂SO₄ and read at 405 and/or 450 nm spectrophotometrically (Molecular Devices, CA).

JAWS II dendritic cell culture: The JAWS II dendritic cell line is an immortalized cell line derived from bone marrow of C57BL/6 mice (ATCC, Manassas, Va.). The cells were maintained in Alpha-modified minimum essential medium (Sigma, St Louis, Mo.) supplemented with 20% fetal bovine serum (FBS), 4 mM L-glutamine, 100 U/ml penicillin, 100 μg/ml streptomycin, 50 μg/ml gentamicin (Invitrogen, Grand Island, N.Y.) and 5 ng/ml of recombinant murine granulocyte macrophage-colony stimulating factor (Peprotech, Rocky Hill, N.J.). (Awasthi et al., 2005). The culture medium was replaced with fresh medium every 48 h.

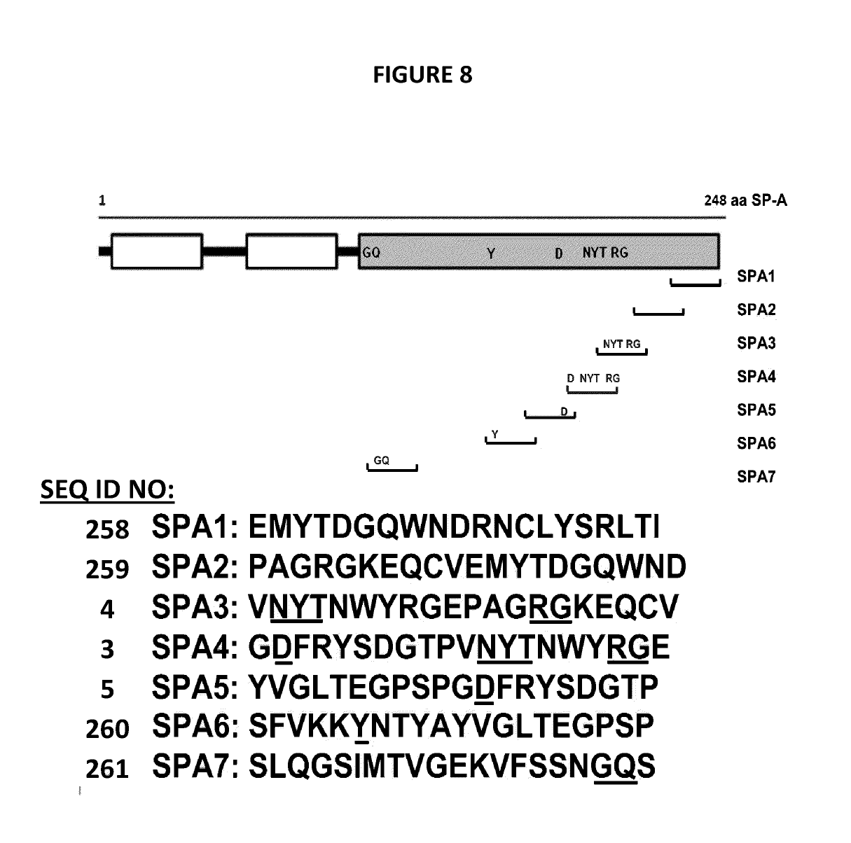

LPS treatment of JAWS II dendritic cells with and without SPA peptides: Based on the results of the bioinformatics analysis (described in Results section), SPA peptides (SPA1 (SEQ ID NO:258); SPA2 (SEQ ID NO:259); SPA3 (SEQ ID NO: 4); SPA4 (SEQ ID NO:3); SPA5 (SEQ ID NO:5); SPA6 (SEQ ID NO:260); and SPA7 (SEQ ID NO:261)) were synthesized. The amino acid sequences were derived from the C-terminal Carbohydrate Recognition Domain (CRD) of human SPA corresponding to the TLR4-interacting sites identified in the in silico model of SPA-TLR4-MD2 complex (FIG. 8). The 20-mer peptides were synthesized by Genscript Corp, NJ; and Mass Spectroscopy and HPLC analyses confirmed the characteristics and purity of synthesized peptides, respectively (data not shown). LAL test (Charles River Lab, MA) confirmed the absence of endotoxin in the peptide samples.

In Example 1, it was demonstrated that short-pulsing of baboon lung dendritic cells with purified lung SPA and recombinant TLR4-MD2 protein leads to TLR4-induced cytokine release against E. coli. Thus, it was questioned if the SPA-derived peptides from the TLR4-interacting regions will demonstrate a similar effect. The JAWS II dendritic cells (1 million) were treated with SPA peptides (1 or 10 μM) with or without E. coli derived LPS (75 ng/ml, highly purified, low protein, Calbiochem, CA). In order to observe the anti-inflammatory properties of the SPA peptides, the cells were treated with peptides for 1 hour prior to addition of LPS (pre-LPS treatment) or after 4 hours incubation with LPS (Post-LPS treatment). The incubation was continued for a total period of 5 hours. Control cells were treated with vehicle, LPS (75 ng/ml), and/or SPA peptides (1 and 10 μM) for a period of 5 hours. The cell-free supernatants were collected and stored at −80° C. for further analysis.

TLR4 expression by immunocytochemistry: The JAWS II-DCs were seeded at a density of 25,000 cells per well of an 8-well chamber slide (Nalge Nunc international, New York, USA). Post-LPS (100 ng/ml) treatment with SPA4 peptide (1 and 10 μM; SEQ ID NO:3) followed, and the cells were fixed for 20 minutes with 3.5% paraformaldehyde prepared in PBS on ice. Permeablization was carried out for 20 minutes on ice with Alpha-MEM medium containing 10% FBS and 0.05% saponin and 10 mM HEPES (Inaba et al., 1998). The cells were washed with PBS supplemented with 1% FBS and 0.05% saponin (wash buffer). Non-specific binding sites were blocked using PBS containing 10% normal mouse serum (Sigma, St Louis, Mo.) for 1 hour at room temperature in a humidified chamber. A rabbit polyclonal antibody to mouse TLR4 (Abcam, Cambridge, Mass.) was added to the cells at a dilution of 1:50 and incubated overnight at 4° C. in a humidified chamber. The cells were washed three times for 5 minutes each, followed by incubation with 10 μg/ml ALEXA FLUOR® 488-labeled donkey anti-rabbit IgG antibody (Molecular Probes, Carlsbad, Calif.) for 1 hour at room temperature in humidified chamber protected from light. The cells were incubated with 100 nM rhodamine-phalloidin (Cytoskeleton Inc, Denver Colo.) for 30 minutes at room temperature. Finally, 1 μg/ml Hoechst 33342 (Molecular Probes, Carlsbad, Calif.) dye was added to the cells. Confocal microscopic images were acquired at Imaging and core facility of Oklahoma Medical Research Foundation using the Zeiss LSM-510META laser scanning confocal microscope. Images were acquired with lens objective of 63× with the x/y stack sizes being 146.2 μm using band pass filter specifications at 435-485, 560-615 and 505-530.

Cytokine (TNF-α) measurement: The TNF-α levels were measured in cell-free supernatants of JAWS II dendritic cells treated with LPS with or without SPA peptides by enzyme linked immunosorbent assay (ELISA) as described earlier (Awasthi and Cox, 2003).

Statistical analysis: The results were analyzed by the Student t-test or ANOVA for statistical significance using Prism software (Graphpad, San Diego, Calif.). At p<0.05, the null hypothesis was rejected.

Results of Example 2:

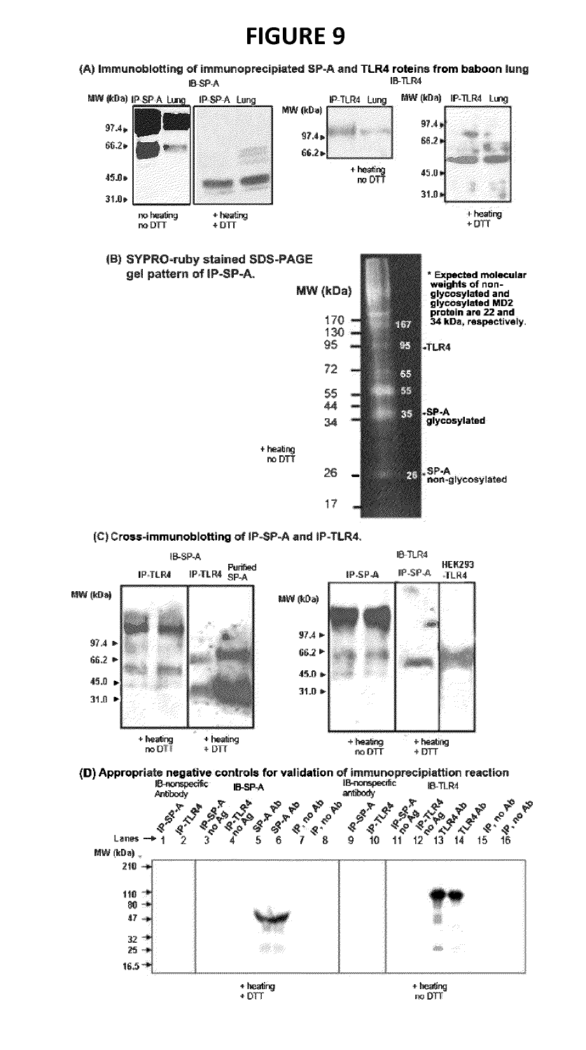

TLR4 and SPA co-immunoprecipitated from baboon lung tissue homogenates. The inventor has previously shown that human-SPA and TLR4-specific antibodies react with baboon-SPA and TLR4 proteins, respectively (Awasthi et al., 1999; Awasthi et al., 2001; Awasthi et al., 2008). Using the same antibody clones, the integrity of SPA and TLR4 was confirmed in baboon lung tissue homogenates. The immunoprecipitation of specific proteins was identified by immunoblotting the IP-SPA and IP-TLR4 eluates from adult baboon lung tissue homogenates using SPA- and TLR4-specific antibodies, respectively (FIG. 9A). The SDS-PAGE gel run of concentrated IP-SPA showed additional protein bands besides SPA, suggesting a number of SPA-binding proteins (FIG. 9B). The lung tissue homogenate protein, lysate protein of HEK293 cells stably-transfected with full-length TLR4, and purified SPA protein were run simultaneously as positive controls to confirm the identity of the IP-SPA and IP-TLR4. The sizes of the TLR4 and SPA protein bands corresponded to the respective proteins in baboon lung tissue homogenates, as published earlier (Awasthi et al., 1999; Awasthi et al., 2001; Awasthi et al., 2008). Neither SPA nor TLR4 was immunoprecipitated when a nonspecific antibody was used in the column (data not shown).

Next, it was hypothesized that if the SPA and TLR4 proteins interact with each other, the two proteins may exist together in the lung and may be co-immunoprecipitated from lung tissue homogenates. The cross-immunoblotting results indicate that SPA and TLR4 are co-immunoprecipitated from baboon lung specimens (FIG. 9C). A major protein band of >100 kDa was identified in both IP-TLR4 and IP-SPA when the IP-eluates were separated on a partially-reducing SDS-PAGE gel and cross-immunoblotted. Protein bands of 34 kDa (similar to SPA monomer) and 66 kDa (SPA dimer) were identified when IP-TLR4 was separated on a reducing SDS-PAGE gel and immunoblotted with anti-SPA antibody (FIG. 9C). A protein band of 55 kDa (TLR4) was recognized when IP-SPA was separated on reducing SDS-PAGE gel and immunoblotted with anti-TLR4 antibody (FIG. 9C). The specificity of the immunoprecipitation reaction was validated using appropriate negative controls (FIG. 9D). These results demonstrated that the IP-TLR4- and IP-SPA-eluates did not contain any noon-specific protein or antibody fractions.

Characterization of purified native baboon lung SPA: To further elucidate the interaction between SPA and TLR4, native SPA protein was first purified from bronchoalveolar lavage fluid specimens of a normal, healthy adult baboon (Awasthi et al., 1999; Awasthi et al., 2001). The purity and identity of the native baboon lung SPA was confirmed by SDS-PAGE and western blotting. Under partially-reducing conditions (heating and no DTT), SPA separated as an oligomer, a 90 kDa-100 kDa trimer, and a 66 kDa dimer on the SDS-PAGE gel. Under reducing conditions (heating+DTT), SPA ran as a 32-34 kDa monomer and a 66 kDa partially reduced dimer. The purified baboon lung SPA protein was also immunoblotted with SPA antibody which identified the SPA-specific protein bands. The solubility of purified baboon lung SPA was 51%. Since the TLR4-MD2 proteins are less abundant in biological systems and distributed throughout, it is difficult to obtain native TLR4-MD2 protein in sufficient quantity. Thus, recombinant human-TLR4-MD2 protein (RnD Systems, MN) was included.

Since TLR4 is a potent receptor for endotoxin, the presence of endotoxin can significantly influence the results. Thus, all solutions and reagents were prepared in endotoxin-free water, and all assays were performed in an aseptic environment. Endotoxin concentration was measured in the purified baboon SPA preparation and in the reconstituted TLR4-MD2 and MD2 proteins by the chromogenic LAL method. The endotoxin concentration was negligible in purified baboon SPA (0.0003 ng/μg protein) and in recombinant TLR4-MD2 and MD2 protein suspensions (≦0.006 ng/μg protein).

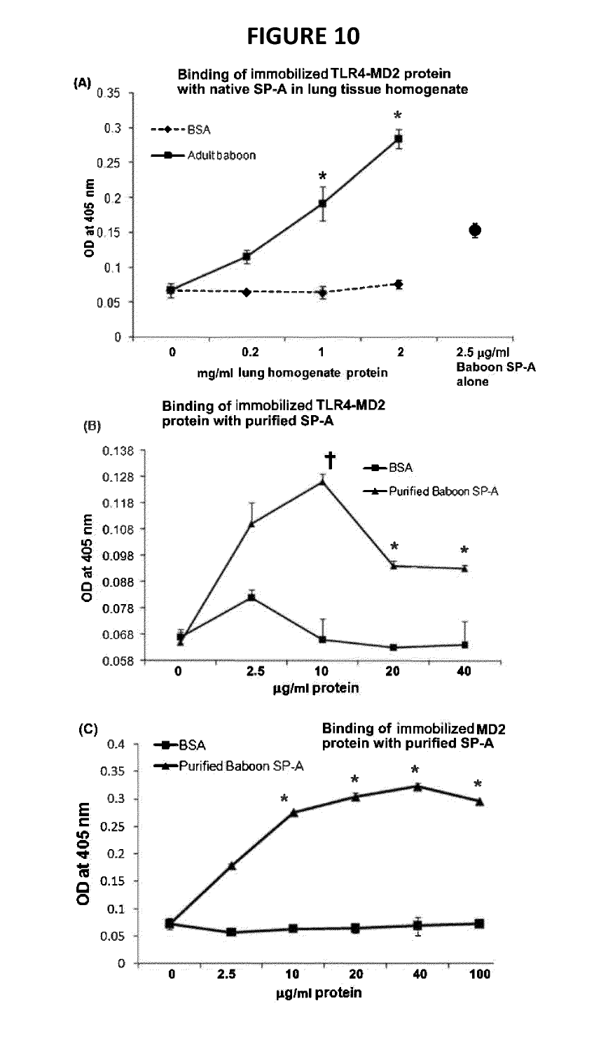

Lung SPA and recombinant TLR4-MD2 proteins interact in vitro. The surface-bound TLR4-MD2 and MD2 proteins showed binding with purified baboon lung SPA and SPA protein present in native form in lung tissue homogenate (FIGS. 10A and 10B). Purified baboon lung SPA was also found to bind to surface-bound MD2 protein (FIG. 10C). In comparison, BSA (negative control) showed negligible binding to the TLR4-MD or MD2 protein.

Protein-protein docking and prediction of interacting amino acids at the interface of SPA-TLR4-MD2 protein complex. In previous sections, the interaction between SPA and TLR4-MD2 proteins was experimentally characterized. In this section, the bioinformatics approaches used to examine the interaction are described. First it is described how data was obtained for bioinformatic analyses; then, the in silico docking of SPA with TLR4-MD2 is described, followed by a description of the rendering of the docking interface.

SPA structure: Under physiological conditions, SPA exists as an octadecamer comprising 6× trimer units, and TLR4-MD2 exists as a dimer. The trimeric crystal structure of neither the human SPA nor the baboon SPA is available in the protein data bank (PDB, www.rcsb.org/pdb). Head et al. solved the crystal structure of the trimeric carbohydrate recognition domain/neck domain of SPA. However, the PDB file and X-ray structure in the protein data bank were available for the monomeric subunit of rat SPA (PDB ID:1R13). Using bioinformatics approaches, it is possible to obtain the structure of trimer by docking three monomers to form a single complex. SymmDock (Schneidman-Duhovny et al., Proteins (2005) 60:224-231; and (2005) Nucleic Acids Res, 33:W363-367), an automated server that deduces the structure of homomultimer with cyclic symmetry when the structure of a monomeric subunit is available, was used for the above task. SymmDock server returned 100 possible trimer complexes that differed in the arrangement of monomers, accompanied by a priority score. Of all the configurations returned by the server, only the top scoring complex was identical to the structure of the trimer shown in the prior art, and the rest had different arrangements.

TLR4-MD2 structure: For TLR4-MD2 proteins, the amino acid sequences and dimer crystal structure of human TLR4-MD2 complex are available in PDB bank (PDB ID: 3FXI). Although the TLR4 and SPA proteins are considered highly conserved proteins, SPA, TLR4 and MD2 sequence homology was checked between the respective animal species using CLUSTALW multiple alignment program (Protein Information Resource, Georgetown University Medical Center, Washington D.C.). Only partial sequences were available for baboon SPA and TLR4, and there was no information available on baboon MD2. The alignment results demonstrate that the SPA, TLR4 and MD2 proteins are highly conserved among different species (including mouse, rat, macaca, baboon and human) (FIG. 11).

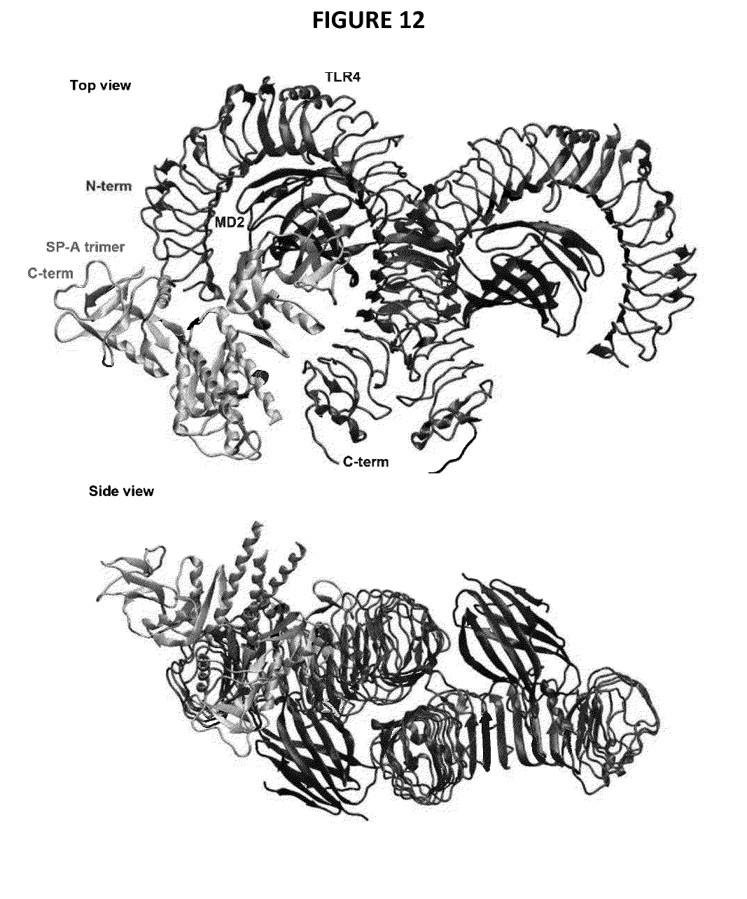

Protein-protein docking: Next, the protein-protein docking was carried out using Global Range Molecular Matching (GRAMM-X) methodology (Tovchigrechko and Vakser (2006) Nucleic Acids Res, 34:W310-314) on a public web server by submitting the PDB files (trimer assembly of SPA and dimer receptor-adaptor molecule complex of TLR4 and MD2). GRAMM-X represents a new implementation of original GRAMM methodology that uses a smoothed Lennard-Jones potential on a fine grid during the global search Fast Fourier Transformation stage, followed by refinement optimization in continuous coordinates and rescoring with several knowledge-based potential terms. The top 100 docked configurations were visually examined to select the most plausible configurations. Results from published studies and the inventor's data were considered to set the inclusion and exclusion criteria for the selection of the most plausible model of SPA-TLR4-MD2 complex. First, 90 configurations that did not show the MD2 adaptor molecule interacting with SPA in the SPA-TLR4-MD2 complex were discarded, because the microwell-based assay results indicated binding between SPA and MD2 adaptor molecule (FIG. 10). In the remaining 10 configurations, some were same configurations with the SPA docked to a different monomer of the TLR4-MD2 dimer. Finally, only three distinct configurations remained. Of these three configurations, the configuration that had the highest area of contact between the molecules was chosen, which also happened to be the configuration ranked ‘one’. It is a model in which the C-terminal portion of SPA binds to the extracellular domain of TLR4 (FIG. 12).

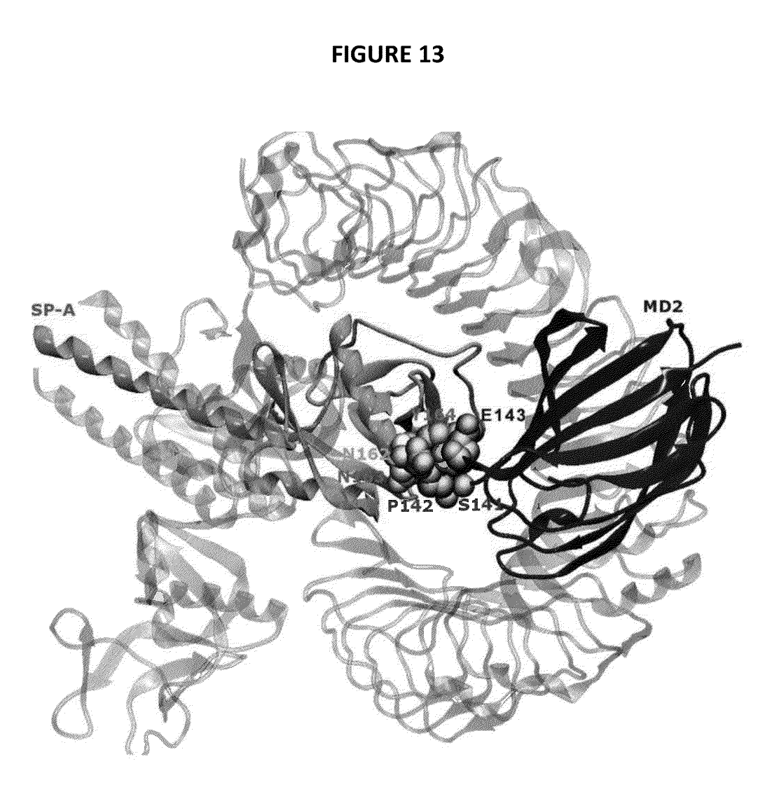

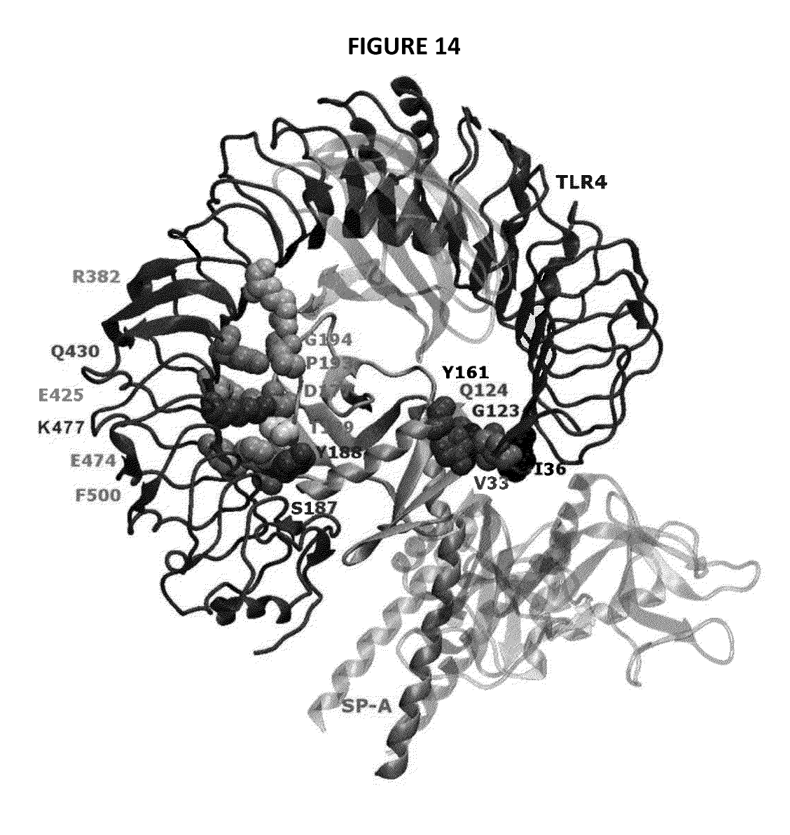

Identification of amino acids at the interface of in silico model of SPA-TLR4-MD2 protein complex: To examine the binding interface of the complex and identify the amino acids at the SPA-TLR4 and SPA-MD2 interfaces, the structures were input into another server called Knowledge-based FADE and Contacts (KFC; comprised of Fast Atomic Density Evaluator (K-Fade): shape specificity features and K-Con: biochemical contacts such as intermolecular hydrogen bonds and atomic contacts) (Darnell et al. (2007) Proteins, 68:813-823). The server predicts the binding hotspots and the associated prediction confidence based on the shape specificity features and biochemical contact features of the residues at the interface. The predicted docking configuration of the SPA-TLR4-MD2 complex with high confidence (K-Fade>0.9 or K-Con>0.9) have been highlighted in FIGS. 13 and 14 using Van der Waals representation. The rendering was carried out using Visual Molecular Dynamics program (Humphrey et al., 1996). The amino acids (SPA: Asn162-Asn163-Tyr164; MD2: Ser141-Pro142-Glu143) in the selected docked configuration were highlighted using a Van der Waals representation (FIG. 13). In the illustration (FIG. 13), the other parts of the complex (two chains of SPA and TLR4) are rendered transparent to focus on the SPA-MD2 interaction site. According to the prediction from the KFC server, the SPA and TLR4 proteins interact at four different places (FIG. 14). The amino acids involved at the interface of TLR4 and SPA (K-Fade >0.9 or K-Con>0.9) are listed in Table 2.

Functional Screening of SPA library revealed a peptide (SPA4; SEQ ID NO:3) that reduces LPS-induced TNF-α secretion. Based on the in silico observations and homology to respective SPA regions between rat and humans, the SPA peptides derived from C-terminal CRD of human SPA were synthesized. SPA peptides were tested for purity by mass spectrometry (Genscript, CA) and for endotoxin contamination by LAL test.

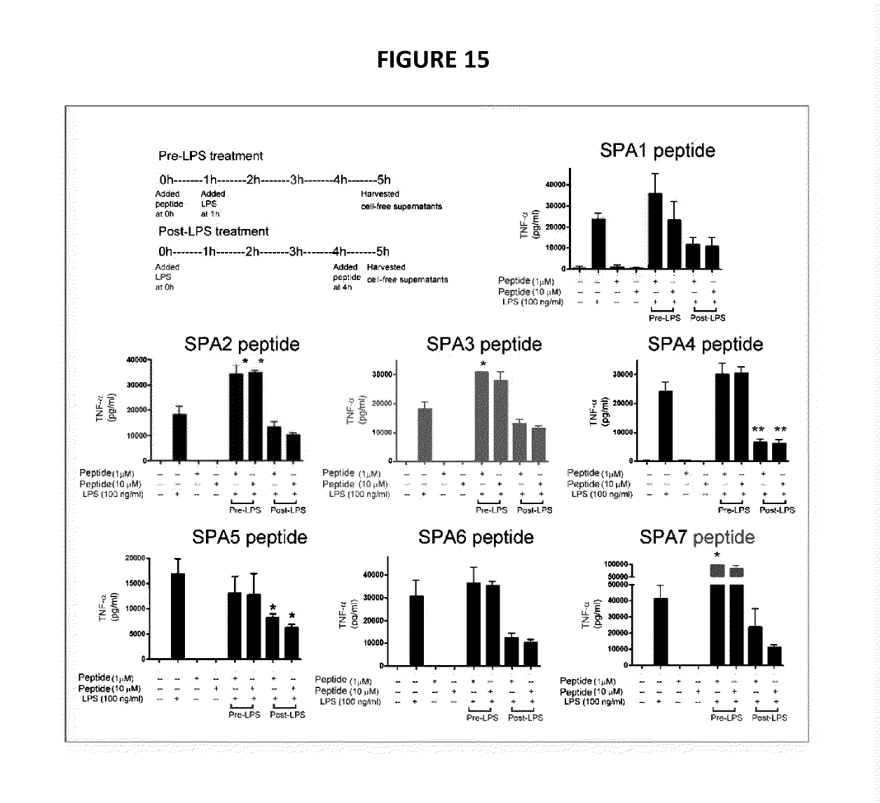

Since an exaggerated activation of TLR4 is directly linked to secretion of pro-inflammatory cytokine (TNF-α) and SPA in downregulating TLR4-induced TNF-α in lung dendritic cells (Example 1), SPA peptides were screened for any changes in LPS-induced TNF-α cytokine secretion in JAWS II dendritic cells. Pre-LPS and post-LPS inflammation models were included in this study to investigate if the peptides affect the LPS-mediated responses, prophylactically or therapeutically. It was found that most of the peptides had no effect on LPS-induced TNF-α secretion in the pre-LPS model, except SPA2 (SEQ ID NO:248), SPA3 (SEQ ID NO:249) and SPA7 (SEQ ID NO:251), which stimulated a slight increase in TNF-α secretion. In the post-LPS model, two peptides (SPA4 and SPA5 peptides; SEQ ID NOS:3 and 5, respectively) were found that inhibited the secretion of TNF-α in post-LPS treated cells at both 1 and 10 μM concentrations (FIG. 15). However, the SPA4 peptide had more effect on LPS-induced TNF-α than SPA5 peptide (mean values 6448 versus 8284 pg/ml at 1 μM concentration, and 6101 versus 6319 pg/ml at 10 μM concentration). Coincidentally, the SPA4 peptide contains most of the amino acids recognized at the interface of SPA and TLR4 in the in silico model of the SPA-TLR4-MD2 complex, and SPA5 peptide contains the first 10 amino acids of the SPA4 peptide.

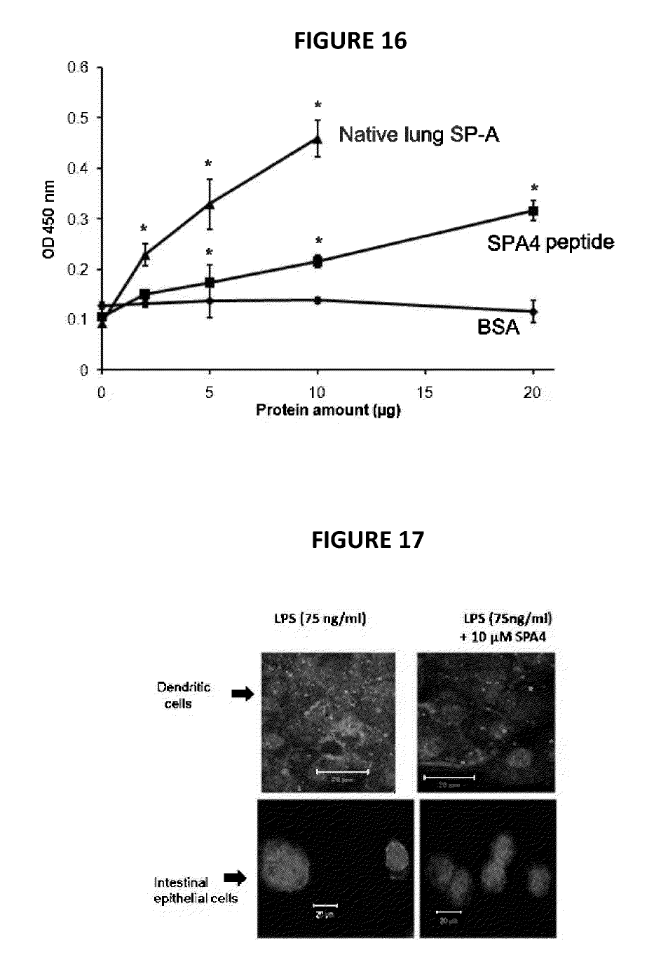

SPA4 peptide binds to TLR4 and blocks the LPS-induced TLR4 expression. Next, the binding of the SPA4 peptide with recombinant TLR4-MD2 protein was confirmed by an in vitro microwell-based binding assay. The binding results showed that similar to purified native SPA, the SPA4 peptide binds to TLR4-MD2 protein (FIG. 16). Binding of the SPA4 peptide to TLR4-MD2 protein was observed as less efficient than the whole native SPA protein, which exists as an octadecamer (composed of six trimers). The SPA4 peptide, however, represents a small portion of the TLR4-interacting region of SPA derived from a monomer. Since a polyclonal antibody was utilized to detect the binding of SPA and SPA4 peptide with TLR4-MD2 proteins, the epitope detection may differ depending on whether it is a fragment (SPA4 peptide) or a full-length protein (purified baboon lung SPA).

TABLE 2
Amino acids identified at the SPA-TLR4 interface.

| Molecule | Amino Acid | Residue # | K-Fade Conf | K-Con Conf |
|---|---|---|---|---|
| TLR4 | VAL (V) | 33 | 1 | 1 |
| (human TLR4) | ILE (I) | 36 | 0.6 | 0.92 |
| | ARG (R) | 382 | 1 | 1 |
| | GLU (E) | 425 | 1 | 1 |
| | GLN (Q) | 430 | 0.93 | 0.92 |
| | GLU (E) | 474 | 0.9 | 1 |
| | LYS (K) | 477 | 0.94 | 0.83 |
| | PHE (F) | 500 | 1 | 0.91 |
| SPA | GLY (G) | 123 | 1 | 1 |
| (rat-SPA) | GLN (Q) | 124 | 1 | 1 |
| | TYR (Y) | 161 | 1 | 1 |
| | ASP (N) | 177 | 0.92 | 1 |
| | SER (S) | 187 | 1 | 0.85 |
| | TYR (Y) | 188 | 1 | 0.81 |
| | THR (T) | 189 | 0.91 | 0.82 |
| | PRO (P) | 193 | 1 | 1 |
| | GLY (G) | 194 | 1 | 1 |

SPA4 peptide-induced changes in the expression of TLR4 were also investigated in dendritic cells. It was found that SPA4 peptide treatment reduced the basal TLR4 expression in JAWS II cells. These results further demonstrate that the LPS-induced TLR4 expression was also suppressed significantly after treatment with SPA4 peptide (* p<0.05, FIG. 17).

Discussion of Example 2:

In lung, SPA is synthesized by type II lung epithelial cells, and is secreted in alveoli as a component of surfactant. SPA plays a critical role in pathogen-opsonization, clearance, downregulation of inflammation, and maintenance of lung function. Earlier the inventor observed that the amounts of SPA secreted in alveoli are significantly reduced in preterm baby baboon having bronchopulmonary dysplasia and in mouse models of lung infection. Thus, it is reasonable to imagine that administration of SPA should enhance clearance of pathogens and inhibit inflammation. Unfortunately, currently available surfactants do not contain SPA, because it is a large and hydrophilic protein and cannot be mixed efficiently with surfactant lipids. Therefore, it is important to search for smaller fragments of SPA. Unavailability of such an SPA-derived fragment has been associated with the lack of an appropriate model to mimic such a complex scenario.

Since the discovery of TLR4 as a pathogen-recognition receptor that is mainly expressed by the antigen-presenting cells, it is now established that an exaggerated expression and activity of TLR4 leads to a deleterious inflammatory response. However, basal activity is important for antigen-presentation and adaptive immunity. Subsequent to finding the reduced levels of SPA, it was observed that the expression of TLR4 is significantly increased in lungs of baby baboons having bronchopulmonary dysplasia. Similar results (i.e., reduction in SPA and increase in TLR4 expression) have also been reported in other models by other investigators. The reduction in SPA amounts and concomitant increase in TLR4 expression corroborates with the clinical condition of patients with lung infection where reduced pathogen-clearance is observed with robust inflammation.

A number of SPA-binding proteins and receptors have been recognized; however their functions and expression by cell type remain unexplored. The binding of SPA to the TLR4 protein has also been recently shown to occur under in vitro conditions; however, the in vivo evidence had been lacking, and functional relevance remained largely unexplored. Example 1 demonstrates that simultaneous pulsing of dendritic cells with SPA and TLR4-MD2 proteins maintains the increased phagocytic uptake, but downregulates the TLR4-MD2-induced inflammatory response against infectious stimuli. It is believed that downregulation of the inflammatory response may be via interaction between SPA and TLR4. Thus, a smaller fragment of SPA containing the TLR4-interacting region should inhibit the TLR4-mediated inflammatory response while maintaining the basic functions of antigen-presenting cells.

In this Example, it was demonstrated that SPA and TLR4 proteins are co-immunoprecipitated from baboon lung tissue homogenates. This is the first report where such an interaction between SPA and TLR4 has been shown to exist in the lung by immunoprecipitation/immunoblotting and microwell-based methods using lung tissue homogenates and purified lung SPA. Earlier, interaction between SPA and TLR4 was studied with purified or recombinant forms of proteins by ligand-blot, microwell-based binding assay and BIAcore methods. Bioinformatics simulation studies further support the interaction between SPA and TLR4-MD2 protein. Although several aspects of TLR4 and SPA binding are not clearly understood, it is clear that the lung microenvironment may significantly influence their interaction. It should be noted that in the antibody-based methods employed here, the kinetics and characteristics of binding between the two proteins depend on the antigen-antibody affinity. The specific binding sites of both the SPA and TLR4 proteins and the kinetic parameters of the native-SPA-TLR4 interaction needed further investigation. It is important to note that the native SPA molecule (ligand) is quite large (octadecamer) because of the oligomerization of trimers, and TLR4 protein is a homomer and associates with other adaptor (MD2) and signaling receptors for its activity. Moreover, it was also found that SPA can bind to the TLR4 adaptor molecule MD2 as well. Thus, computer modeling of the SPA-TLR4-MD2 complex was considered; an in silico model of SPA-TLR4-MD2 complex was obtained where the binding features fitted best with the results from immunobiochemical assays (FIGS. 9 and 10). The selected in silico model was analyzed further to identify potential binding sites and amino acids.

As identified earlier, the functional significance of such an interaction is very difficult to assess in vivo. The functional relevance of such an interaction can be better examined under in vitro conditions in a controlled environment using cell culture systems and the appropriate dosage of effector molecules. Thus, the JAWS II dendritic cell system, established in the inventor's lab, was used to investigate the effects of SPA peptides derived from the TLR4-interacting region on cytokine response against a well-known inflammatory stimuli: LPS. It was found that SPA4 peptide (1) encodes most of the amino acids belonging to TLR4-interacting region in in silico model, (2) binds to TLR4-MD2 protein, and (3) reduces LPS-induced TLR4 expression and cytokine response.

These results demonstrate that SPA blocks the TLR4-MD2-mediated intracellular signaling and cytokine release against infectious stimuli. Recently in human monocytes culture system, Henning et al. found that SPA did not affect TLR4 expression, but it downregulated the TLR4-mediated signaling against LPS. However, based on the information on the interacting amino acids at the SPA-TLR4 interface in the computer-simulated SPA-TLR4-MD2 complex model, and screening of the peptides, one peptide was identified (SPA4) that not only inhibits the LPS-stimulated TLR4 expression, but also suppresses LPS-induced TNF-α release.

### Example 3

### Use of SPA4 to Modulate TLR4 Signaling for Treatment of Intestinal Inflammation

About a million people are currently suffering from inflammatory bowel diseases (IBD) in the US alone, and new cases are being diagnosed at the rate of 10 cases per 100,000 people (American College of Gastroenterology). IBD causes chronic inflammation in the intestine with alternating periods of active and latent disease, and accounts for a huge economic cost associated with multiple clinic visits and hospitalizations. Chronic inflammation can lead to debilitating complications including colon cancer. Lifelong pharmacotherapy remains the mainstay of IBD management, whereas surgery is indicated for the treatment of refractory disease or specific complications.

Conventional IBD therapies include the use of aminosalicylates, corticosteroids and immunosuppressive drugs (e.g., methotrexate, cyclosporin A). These traditional treatment modalities provide symptomatic relief to some extent depending on the severity of the disease, but exert numerous side effects. The side effects can range from perturbed physiological functioning of important organ systems to potentially fatal opportunistic infections. However, recently a better understanding of the mucosal immune system and genetics involved in the pathogenesis of IBD led to development of biologic medications. These medications include infliximab, adalimumab and certolizumab pegol, which are antibodies to block TNF-α, an inflammatory cytokine that is present in increased amounts in patients with IBD. Adverse reactions with these anti-TNF-α products include infusion or injection site reactions, upper respiratory infections and malignancies. Other new biologics that have recently entered into clinical trials include adhesion molecule inhibitor (Natalizumab), anti-IL-12, IFN-γ antibodies and growth factors. As the safety and toxic effects remain to be completely evaluated for these new biologics, Natalizumab has already been temporarily discontinued due to JC virus brain infections. At a molecular level, these inhibitors and antibodies target the pre-formed or secreted inflammatory cytokines or cell-surface molecules that can provide neither cure nor a durable effect after discontinuation. Well thought-out strategies are needed to design novel treatment modalities that can provide more sustained therapeutic effect without any significant toxicity or side effects.

Toll-like receptor-4 (TLR4) was first discovered in 1996 as an innate immune recognition receptor for Gram negative bacterial lipopolysaccharide (LPS). Besides LPS, TLR4 is now known to recognize endogenous inflammatory signals, such as heat-shock proteins, fibronectin, etc. Over a period of the last 15 years, since the discovery of TLR4, a great deal about its critical role in inflammatory responses in infectious and non-infectious diseases has been determined. Since inflammation is a hallmark of IBD, TLR4 is thought to be important. It has been recently hypothesized that TLR4-signaling probably serves a dual role in the gut as a mediator of both inflammation and mucosal repair. A hypothetical model was provided suggesting that basal TLR4-signaling is required for normal functioning and intestinal homeostasis. It is the exaggerated TLR4-signaling in response to physiological stressors (e.g., hypoxia) and infectious stimuli (e.g., LPS), that leads to intestinal inflammation. Thus, the novel therapeutic agents that can block this exaggerated TLR4/TLR4-signaling may eventually suppress the inflammatory response and help alleviate the symptoms of IBD.

Results of Example 3

SPA4 peptide (SEQ ID NO:3) inhibits the LPS-induced TLR4 expression in dendritic cells and SW480 intestinal (colonic) epithelial cells. After the shorter SPA fragment (SPA4) was designed that showed binding to TLR4-MD2 protein (Example 2), its immunomodulatory effects were studied using well-established dendritic (JAWS II; ATCC, VA) (Vilekar et al., 2010) and intestinal epithelial cells (SW480; ATCC, VA). Both of these cells constitutively express the TLR4 gene; the protein expression is low in dendritic cells under basal conditions. LPS treatment induced TLR4 expression in both cell types. First, it was determined if the SPA4 peptide inhibits LPS-induced expression of TLR4 in these cells. The cells were treated with 75 ng/ml or 1 μg/ml E. coli-derived LPS (Calbiochem, CA; highly purified, low protein, does not activate TLR2 signaling) for 4 hours prior to addition of SPA4 peptide. After a total of 5 hours, TLR4 expression was studied by confocal microscopy.

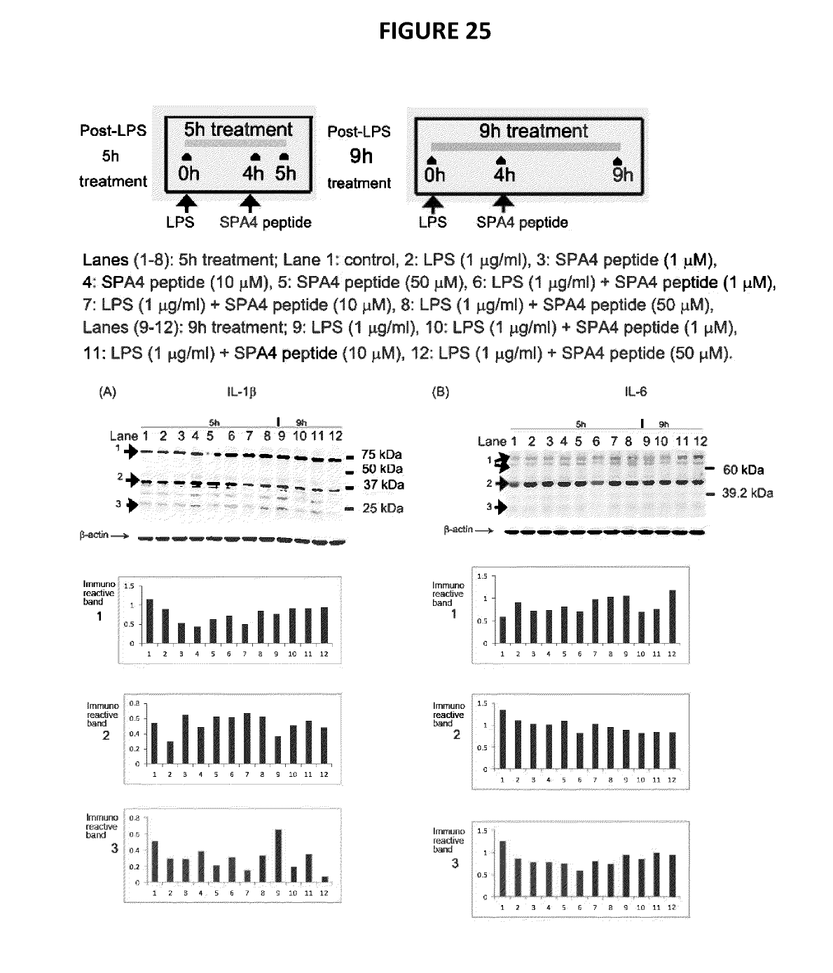

The results demonstrated that the SPA4 peptide reduced LPS-induced TLR4 expression to a basal level (FIG. 17) and inhibited the secretion of pro-inflammatory cytokines (FIGS. 15 and 25). These effects were not related to any cell toxicity, since the SPA4 peptide does not affect the viability of cells within this time period.

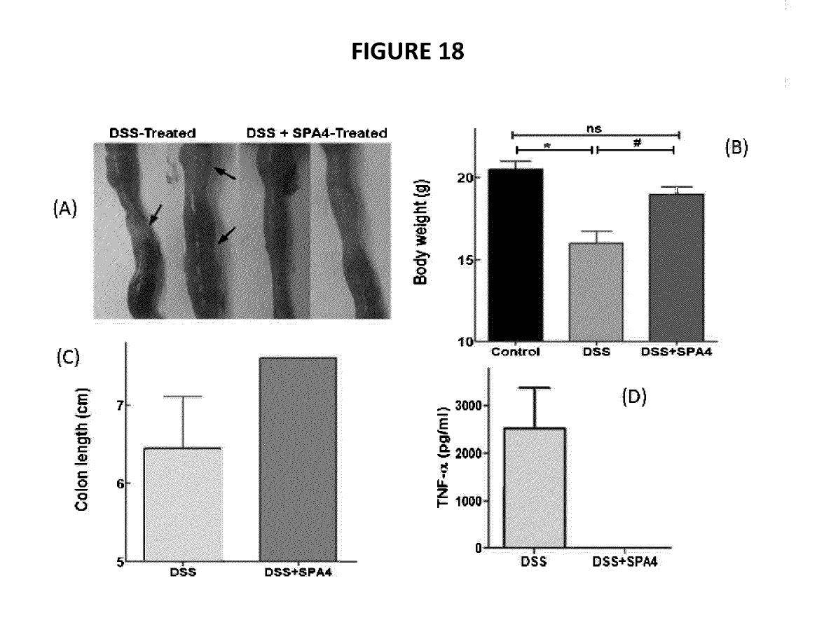

SPA4 peptide reduced serum TNF-α and inflammation in Dextran sodium sulfate (DSS)-colitis model in mice (FIG. 18). SPA4 peptide was also evaluated in the mouse model of DSS-colitis. DSS (3% in drinking water) was given to the mice for a period of 7 days. In the treatment group, mice were simultaneously injected with SPA4 peptide (100 μg daily via intraperitoneal route). After 2 days of recovery period, the mice were weighed, colons were macroscopically examined, and blood samples were harvested. The TNF-α levels were measured by ELISA.

FIG. 18 demonstrates that SPA4 peptide reduces inflammation in the DSS-colitis model. Mice with colitis lost about 25% body weight; in addition, their colons were distended and shortened. Also, increased levels of circulating TNF-α were detected in the serum. As shown in FIG. 18A, simultaneous treatment with SPA4 peptide reduced the amounts of distension and shortening of the colon, thus demonstrating that the SPA4 peptide reduced the colitis symptoms. In addition, FIGS. 18B and 18C demonstrate that simultaneous treatment with the SPA4 peptide recovered body weight (18B) and colon length (18C) when compared to the DSS-colitis mice. Finally, FIG. 18D demonstrates that SPA4 peptide treatment completely inhibited the DSS-induced serum levels of circulating TNF-α.

Therefore, this Example has demonstrated that the SPA4 peptide suppresses TLR4-signaling in intestinal epithelial and immune cells under inflammatory stress conditions. This suppression of TLR4 at the cellular level results in reducing intestinal inflammation in animals.

### Example 4

### SPA4 Inhibits Lipopolysaccharide-Stimulated Inflammatory Responses, Migration, and Invasion of Colon Cancer SW480 Cells

Colorectal cancer is the third most common cancer and leading cause of cancer-related mortality in the United States. As per a recent annual report, 141,210 new cases of colorectal cancer and 49,380-associated-deaths were reported in the US only (National Cancer Institute at the National Institute of Health, Washington D.C.). An exaggerated inflammatory response has been reported to increase the risk of colorectal cancer in patients with inflammatory bowel disease (IBD), ulcerative colitis (UC) or Cohn's colitis.

This inflammation-induced progression of cancer can potentially be suppressed by anti-inflammatory agents. Common anti-inflammatory medications include the use of aminosalicylates, corticosteroids and immunosuppressive drugs (e.g., methotrexate, cyclosporin A). While it remains to be established whether conventional anti-inflammatory agents can have chemopreventive effects against cancer, an understanding of mucosal immune system and genetics has led to the recent advancements in development of biologic medications against IBD and cancer, specifically anti-TNF-α products (Bosani et al., 2009). A number of anti-TNF-α products (antibodies and receptor antagonists) have been approved by the FDA for reducing inflammation in patients with colitis. Traditional medications and biologics provide only transient relief and have significant side effects that include increased risk of infections and perturbed physiological functioning of important organ systems. These treatment strategies provide short-lived symptomatic relief, mainly because these products act against already secreted TNF-α cytokine or other chemical mediators.

Key molecules involved in inflammatory pathways include Toll-like receptors (TLRs), nuclear factor (NF)-kB, cytokines, growth factors, kinases, cyclooxygenases and nitric oxide synthases. TLRs are unique because they not only sense the “danger signals” in the form of infectious agents or stress-ligands, but by the virtue of their intracellular Toll/Interleukin-1 receptor (TIR) domain, the TLRs are associated with a complex intracellular signaling network, including NF-κB-inflammatory pathway. Thus, new therapies targeting TLR may be of benefit in suppressing inflammation in more sustained fashion. Among a number of TLRs, Toll-like receptor-4 (TLR4) was first discovered in 1996 as an innate immune recognition receptor for Gram-negative bacterial lipopolysaccharide (LPS). TLR4 is now well-recognized as pattern-recognition receptor against a diverse array of ligands including endogenous stress ligands or damage-associated molecular patterns (DAMPS), such as but not limited to, heat-shock proteins, fibronectin, etc. A number of recent studies have reported the involvement of TLR4 in colitis and cancer progression. Constitutive activation of TLR4 augments inflammatory response in colitis-induced tumorigenesis. Colon cancer cell lines SW480 and SW620 constitutively express TLR4. In SW480 cells, LPS treatment induces cytokine synthesis/secretion, cell-migration and adhesion. Increased cell migration and adhesion are hallmarks of tumor growth and metastasis. Thus, the inventor postulated that suppression of LPS-stimulated TLR4-signaling will help control inflammation and inflammation-induced metastatic property of SW480 cells. Presumably, a therapeutic that inhibits intracellular inflammatory signaling is expected to exert sustained anti-inflammatory effects and help prevent inflammation-induced cancer.

The previous Examples describe the identification of the TLR4-interacting SPA4 peptide, as well as its ability to reduce secretion of TNF-α by a dendritic cell line in response to LPS stimuli. In this Example, the ability of SPA4 peptide to inhibit the LPS-induced TLR4-NF-κB signaling and resulting inflammatory response in SW480 colon cancer cell line was studied. Simultaneously, the effects of SPA4 peptide on migration, viability and cell cycle progression of SW480 cells were also investigated.

Materials and Methods for Example 4:

Cell culture: Human colorectal adenocarcinoma cells: SW480, derived from the colon of a cancer patient (original stock from ATCC, VA), were obtained from the laboratory of Dr. Shrikant Anant (University of Kansas Medical Center, Kansas City, Kans.). The cells were maintained in Dulbecco's minimum essential medium (D-MEM, Invitrogen, CA), supplemented with high glucose (4.5 g/l D-glucose), sodium pyruvate (1 mM), L-glutamine (4 mM), fetal bovine serum (10%) and antibiomyco (1%, Invitrogen, CA). Cells were maintained at 37° C. in a humidified 5% CO₂ incubator.

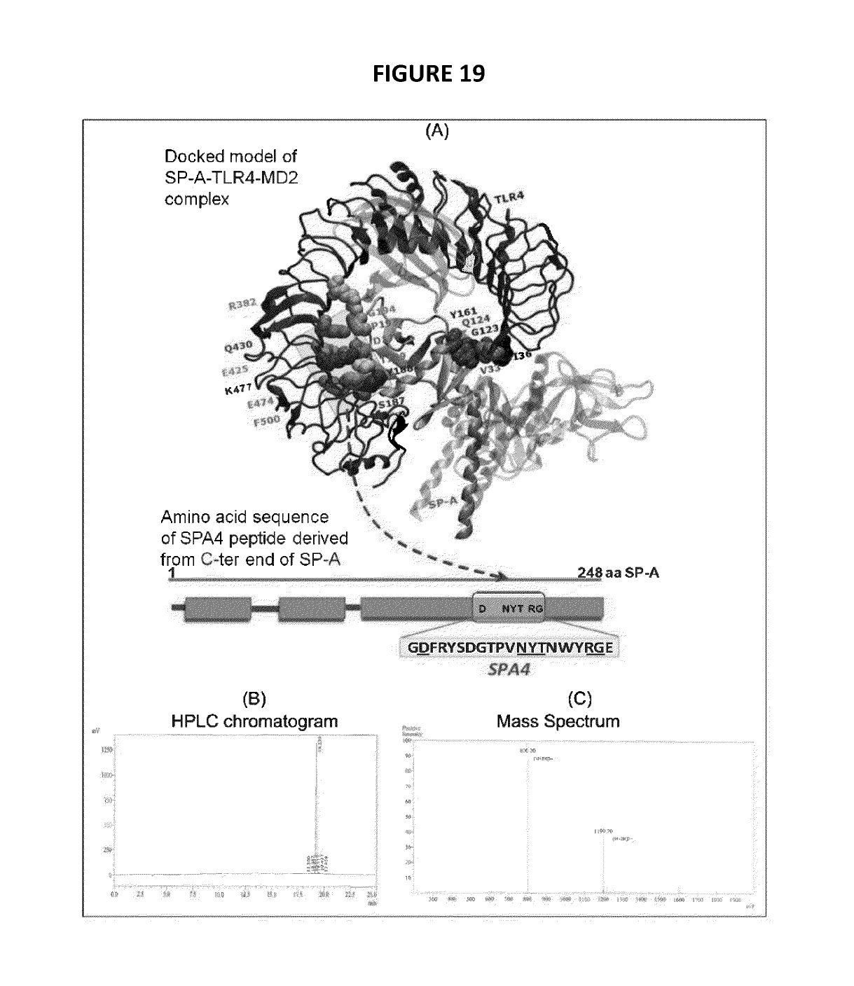

SPA4 peptide: The 20-mer SPA4 peptide (amino acid sequence: GDFRYSDGT PVNYTNWYRGE; SEQ ID NO:3) derived from the C-terminal region of SPA, was synthesized by Genscript (Piscataway, N.J.). The mass spectroscopy and high-performance liquid chromatography were performed on all the batch-preparations of the SPA4 peptide to confirm its purity (FIG. 19). The peptide was suspended in endotoxin-free HyClone Cell culture grade water, and endotoxin content was measured by Limulus Amoebocyte Lysate (LAL) assay (Charles River Laboratories International, Inc., Wilmington, Mass.).

Measurement of n-octanol/water partition coefficient (K_{o/w}) of SPA4 peptide: The n-octanol/water partition coefficient (K_{o/w}) is a measure of hydrophobicity/hydrophilicity. It is calculated as the ratio of the concentration of a chemical in n-octanol to that in water in a two-phase system at equilibrium. An equal volume of MiliQ water and n-Octanol was mixed in a microcentrifuge tube, and shaken for 4 hours at 25° C. Weighed amount of the SPA4 peptide was then added to this n-Octanol-water mixture and shaken overnight at 25° C. The SPA4 peptide-n-Octanol-water mixture was allowed to settle for 2 hours. The aqueous phase was separated by centrifugation at 16,000×g for 10 minutes. The concentration of SPA4 peptide in aqueous phase was measured by spectrophotometric absorbance readings at 280 nm. The concentration of the SPA4 peptide in n-Octanol phase was obtained after subtracting the amount of peptide in water phase from that of the total SPA4 peptide added. Finally, K_{o/w} of SPA4 peptide was determined using following formula:

$${K}_{o / w} = \frac{concentration of SPA 4 peptide in octanol phase}{concentration of SPA 4 peptide in aqueous phase}$$

Binding of SPA4 peptide to LPS: The binding of SPA4 peptide to LPS was studied by LAL assay as per the method described by Giacometti et al. (2004). Briefly, 0-20 μM SPA4 peptide or polymyxin B (positive control) solutions were added to 0.01 ng/ml Escherichia coli O111:B4 LPS (supplied with the kit, Charles River Laboratories International, Inc., Wilmington, Mass.) in the wells of a 96 well plate and incubated at 37° C. for 40 minutes. Fifty μl of LAL substrate solution was then added to each well, and the plate was incubated for another 10 minutes. Finally, substrate-buffer solution was added, and optical density readings (OD) were obtained at 405 nm after 0, 6 and 12 minutes of addition of substrate. ΔOD values for SPA4 peptide or polymyxin B incubated with LPS (ΔODₜᵣₑₐₜₘₑₙₜ), LPS alone (ΔOD_{LPS}) and blank (ΔOD_{Blank}) were calculated by subtracting the OD values obtained at 6 or 12 minutes from those obtained at 0 minutes. Percent binding of SPA4 peptide and polymyxin B was calculated at 6 and 12 minutes of reaction using following formula:

$$Percent binding = \frac{1 - ( Δ {OD}_{treatment} - Δ {OD}_{blank} )}{Δ {OD}_{LPS} - Δ {OD}_{Blank}} \times 100$$

Expression of TLR4: Next, the effect of SPA4 peptide on the expression of TLR4 in SW480 cells was investigated by immunocytochemistry and laser confocal microscopy. Briefly, about 2.5×10⁴ cells were seeded in an 8-well chamber slide (Thermo Fisher Scientific, Rochester, N.Y.) in complete medium. The cells were treated with E. coli O111:B4-derived, highly-purified, low-protein LPS (100 ng/ml or 1.0 μg/ml; Calbiochem, CA) for 4 hours following 1 hour incubation with SPA4 peptide (1, 10 and 100 μM). The cells were fixed in 3.5% paraformaldehyde in Dulbecco's PBS (DPBS) and permeabilized with 0.05% saponin solution (Inaba et al. 1998). The wells were washed with DPBS supplemented with 1% FBS and 0.05% saponin, and stained with TLR4-specific antibody (1:50 dilution, Abcam, MA) and 10 μg/ml ALEXA FLUOR® 488-labeled secondary anti-rabbit IgG antibody (Molecular Probes, Inc., Eugene, Oreg.). After washing, the cells were stained with 100 nM rhodamin-phallodin (Cytoskeleton Inc, CO) and 1 μg/ml Hoechst 33342 dyes (Invitrogen-Molecular Probes, CA). Confocal microscopic images were acquired at the Imaging core facility of the Oklahoma Medical Research Foundation, Oklahoma City, using the Zeiss LSM-510 META laser scanning confocal microscope. Images were acquired with lens objective of 63× with the x/y stack sizes being 146.2 μm using band pass filter specifications at 435-485, 560-615 and 505-530.

NF-κB activity: Since binding of LPS to TLR4 activates NF-κB through MYD88-dependent and independent pathways, the effects of SPA4 peptide on basal and LPS-induced NF-κB activity were investigated in SW480 cells transfected with a dominant negative construct of MYD88 (MYD88DN) and NF-κB firefly-luciferase reporter plasmid DNA. Both the short- (1 hour) and long-term (5 hours) effects of the SPA4 peptide on NF-κB activity were studied.

The SW480 cells were transiently-transfected with NF-κB firefly-luciferase reporter plasmid pGL4.32 (luc2P/NF-κB-RE/Hygro, Promega, WI; provided by Dr. Kelly Standifer, Department of Pharmaceutical Sciences, University of Oklahoma Health Sciences Center, Oklahoma City, Okla.) and MYD88-dominant negative plasmid construct (MYD88DN, provided by Dr. Ruslan Medzhitov, Yale University, CT). MYD88 dominant negative plasmid DNA construct lacked the death domain and intermediate domain (Medzhitov et al. (1998) Mol Cell, 2:253-258). Briefly, NF-κB-Luciferase reporter and MYD88-dominant negative plasmids (1.0 μg each) were mixed with 6 μl of FuGENE® HD reagent (Roche, IN) in 92 μl of serum-free low-glucose DMEM medium (Invitrogen, CA), and incubated for 20 minutes at room temperature. The transfection-mix was then added to the SW480 cells. The SW480 cells transfected with a plasmid DNA construct expressing enhanced green fluorescent protein (pHYG-EGFP; Clontech, CA) were observed under Leica DM4000B fluorescent microscope. The transfection efficiency was calculated as percent of cells expressing EGFP over total number of cells in the brightfield channel. An empty vector plasmid DNA (pcDNA 3.0; obtained from Dr. Brian Ceresa, Department of Cell Biology, University of Oklahoma Health Sciences Center, Oklahoma City, Okla.) was used as negative control. The cells were incubated for 18-20 hours at 37° C. in humidified 5% CO₂ chamber. After the completion of incubation period, fresh complete medium was added to the cells. Cells were then challenged with LPS (1.0 μg/ml) for 4 hours following the treatment with SPA4 peptide (1, 10, 50 μM) for 1 hour (total period of 5 hours; short-term treatment model) and 5 hours (total period of 9 hours; long-term treatment model). The LPS remained in the medium throughout the incubation period.

After completion of the incubation period, the medium supernatants were removed and cells were washed with room-temperature DPBS. The cell extracts were prepared using the reporter assay cell-lysis buffer (Promega, Fitchburg, Wis.) and stored at −80° C. for further analysis. The firefly-luciferase activity (measurement of NF-κB activity) was measured using the luciferase reporter assay system (Promega, Fitchburg, Wis.). Briefly, 50 μl of luciferase assay reagent was added to the 20 μl cell lysate by automated dispenser of Synergy HT multi-mode microplate reader (Biotek, Winooski, Vt.), and luminescence was read within 10 seconds. Total protein content in cell lysates was estimated using BCA protein assay kit (Pierce, Rockford, Ill.). The luciferase activity units were finally normalized with the total protein content of cell lysates.

Expression of NF-κB pathway molecules by immunoblotting: The expression of NF-κB pathway molecules (inhibitor kappa-Bα: IKBα, phosphorylated IKBα, p65, phosphorylated p65, RelB and COX-2) in SW480 cell-lysates treated with LPS±SPA4 peptide was studied by immunoblotting. For the immunoblotting, 10 μg of total cell-lysate proteins were fractionated on Novex 4-20% Tris-glycine gradient SDS-PAGE gel (Invitrogen, Carlsbad, Calif.) by electrophoresis. Separated proteins were electro-transferred onto a nitrocellulose membrane using iBlot gel transfer device (Invitrogen, Carlsbad, Calif.). The non-specific sites were blocked by incubating the membrane with 7% skimmed milk in Tris-buffered saline with 0.4% TWEEN®-20 (TBST) for 1 hour at room temperature. The blocked membranes were incubated overnight at 4° C. with 1:1000 diluted anti-human antibodies against NF-κB canonical pathway molecules: phosphorylated inhibitor kappa-Bα (IKBα), total-IKBα, p65, RelB (Cell Signaling Technology, Inc., Danvers, Mass.), phosphorylated p65 (Santacruz Biotech, Inc., Santa Cruz, Calif.) and cyclooxygenase-2 (COX-2; Santacruz Biotech, Inc., Santa Cruz, Calif.). The membranes were washed with TBST and incubated at room temperature for 45 minutes with 1:3500 diluted horse-radish peroxidase (HRP)-conjugated-secondary antibody (Sigma-Aldrich, St. Louis, Mo.). The immunoreactive bands were detected by Super Signal West Femto detection reagent (Thermo Fisher Scientific, Barrington, Ill.). In order to ensure equal protein loading in the wells, the membranes were stripped of probing antibodies at 60° C. for 45 minutes using a stripping solution containing 10% SDS, 0.5 M Tris and β-mercaptoethanol (35 μl/ml), and re-probed with anti-actin antibody (Sigma-Aldrich, MO; 1:1000 in TBST). The immunoblots were imaged using the Ultraquant image acquisition program (UltraLum Inc., Claremont, Calif.). The densitometric readings were obtained for immunoreactive bands with Image J 1.42q program (NIH, USA). Finally, arbitrary densitometric values for proteins of interest were normalized with those of β-actin.

Expression of IL-1β and IL-6: The post-LPS treatment model was utilized for assessing the effects of SPA4 peptide on LPS-induced cytokines: IL-1β and IL-6. Post-LPS treatment models (short-term and long-term) are described above. The cell-lysates were prepared either in commercially available cell-culture lysis reagent (Promega, WI) or directly into SDS-PAGE sample buffer containing 50 mM dithithreitol (Cell Signaling Technology, MA). Ten μg of cell-lysate proteins were separated on 4-20% Novex Tris-glycine gradient SDS-PAGE gel (Invitrogen, CA) or 10% separating-5% stacking acrylamide gel. The expression levels of cytokines were measured by immunoblotting as described above using 1:1000 diluted antibodies against IL-1β and IL-6. Both antibodies were purchased from Santacruz Biotech, CA.

Cell migration: SW480 cells were plated in 30 mm tissue-culture-treated dishes at a density of 1.0×10⁶ cells per plate. At 80-90% confluence, a “reference line” was drawn at the bottom of the plate. The cells were scratched off from one side of the reference line using a rubber policeman. A picture was taken at 0t that helped in marking the “start line”. Cells were then washed with complete medium and incubated with LPS (1.0 μg/ml) and/or SPA4 peptide (1, 10 and 50 μM). Photomicrographs of cells migrated across the “start line” were taken in different fields after each treatment at 24, 48, and 72 hours (±2 hours) following traceable inscriptions made under the plate at three different points, with a Canon digital camera. On 24, 48 and 72 hour images, a second line was drawn along the edge of cells to represent the migration of cells. Cell migration was calculated by measuring the distance cells migrated from the “start line”. Only the continuous migration of cells was considered for measurement. The islets of cells were disregarded. The cell migration was calculated using the following formula: (distance between “start line” at 0 h−“reference line”)−(distance between “72 hours line”−“reference line”).

Cell cycle analysis: The effect of SPA4 peptide on cell cycle progression was studied by flow-cytometry. About 500,000 cells were seeded per well into 6 well plates. The cells were challenged with LPS (100 ng/ml) for 4 hours. After the completion of 4 hours LPS-challenge period, SPA4 peptide (10, 50, and 100 μM) was added to the cells. Cells were further incubated for 20 and 40 hours. Vehicle-treated cells were also included. After 20 and 40 hours of total incubation period, both adherent and non-adherent cells were collected and centrifuged at 260×g for 5 minutes. The supernatant was discarded and cell pellet was washed with DPBS (Invitrogen-Gibco, NY). The cells were fixed in 70% ice-cold ethanol on ice for 1 hour and stained with a buffer containing 200 μg/ml DNase-free RNase A (Sigma, St Louis, Mo.), 0.1% v/v Triton-X 100 and 20 μg/ml propidium iodide (Molecular Probes, Carlsbad, Calif.). The cells were incubated at 4° C. for 30 minutes in the dark, before measuring cell fluorescence using Becton Dickson FACS Calibur flow cytometer. The single cells were selected by gating out the aggregates and the percent number of cell populations in different cell cycle phases were calculated by de-convoluting the results ModFIT software (Verity software house, Topsham, Me.).

Cell Invasion: The effects of the SPA4 peptide on LPS-induced invasiveness of SW480 cells were studied by a modified Boyden chamber Matrigel method using 8 μm transwell chambers. The insert wells were prepared by rehydrating the Matrigel matrix with DMEM medium for 2 hours at 37° C. The rehydration solution was carefully removed, and DMEM medium containing antibiotics and 10% FBS was added to the bottom of the insert. The cells (125,000 cells per well) suspended in DMEM medium containing antibiotics and 1% FBS were added onto the top of the insert with 8 μm filters (BD biosciences, MA). The LPS (1 μg/ml) was added onto the top of the insert at the time of seeding the cells. After 4 hours of incubation with LPS, cells were treated with SPA4 peptide (1 μM and 10 μM). After 96 hours of incubation, the medium was removed from the inserts, and the top layer of Matrigel was scrubbed. The inserts were removed, and cells at the bottom of the inserts were stained with Diff-Quik Wright-Giemsa stain as per the manufacturer's instructions (Dade Behring, IL). Stained cells at the bottom of the insert were observed under microscope using 20× objective. Multiple representative photomicrographs were taken for each well and the numbers of cells invaded through the matrix were counted.

Cell viability: The TLR4-NF-κB signaling can have multifaceted implications, including the effects on proliferation and viability of SW480 cells. Thus, the effect of SPA4 peptide on viability of SW480 cells was studied. The cells (250,000 and 500,000 per well) were seeded into 24 well plates and treated with SPA4 peptide (10, 50, and 100 μM) after LPS-challenge (100 ng/ml) for 4 hours. The LPS remained in the medium throughout the incubation period thereafter. The cells were collected after 3-5 days of treatment and stained with propidium iodide (1 μg/ml) for 20 minutes on ice. The stained cells were run on a flow-cytometer (Accuri flow cytometer, MI), and propidium iodide staining was assessed on FL2 channel. The histograms were obtained for each treatment using C Flow Plus software and compared between the groups. The % number of cells exhibiting positive (dead cells) and negative (live cells) propidium iodide staining were noted. Untreated cells and LPS-treated cells served as controls.

Statistics: The results were analyzed by one way Analysis of Variance (ANOVA) using a statistical analysis program (Graphpad Prism, CA). A p-value of <0.05 was noted, otherwise indicated.

Results of Example 4:

Characteristics of SPA4 peptide: The SPA4 peptide batch preparations were always tested for purity by HPLC chromatography and Mass spectrometry (FIG. 19) or any contamination with endotoxin by LAL assay. The endotoxin level was undetectable (below the lower limit of 0.001 ng/ml) in reconstituted SPA4 peptide suspensions of all the batches.

As per the computer simulation Solvent AccesiBiLitiEs (SABLE) program (Division of Biomedical Informatics, Children Hospital Research Foundation, Cincinnati, Ohio), the SPA4 peptide is predicted to have coiled and beta strand structures. Relative solvent accessibility (RSA) of 11 amino acid residues is above 25% (FIG. 20), suggesting that the SPA4 peptide is easily soluble in water. Furthermore, the Ko/w partition coefficient of the SPA4 peptide was measured. The Ko/w partition coefficient of SPA4 peptide was 0.56. These results further confirm that the SPA4 peptide is hydrophilic in nature.

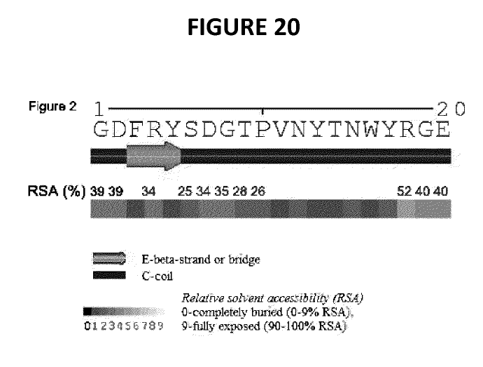

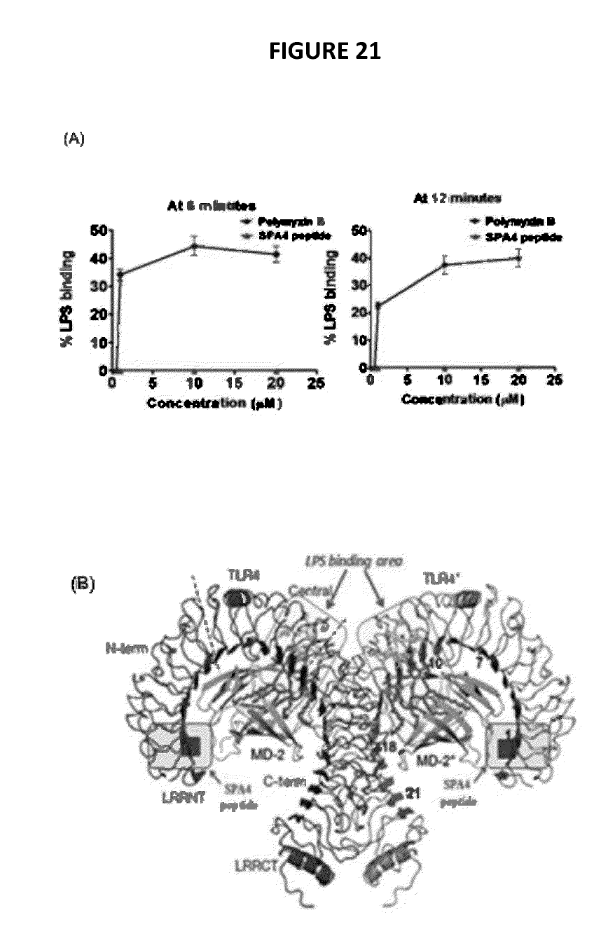

SPA4 peptide does not bind to LPS. Example 2 illustrated that the SPA4 peptide binds to recombinant TLR4-MD2 protein. In order to further validate that the anti-inflammatory effects of SPA4 peptide are not through the binding of SPA4 peptide to TLR4-ligand LPS, the binding of SPA4 peptide to LPS was studied in vitro. The results in FIG. 21A show that the SPA4 peptide does not bind to LPS.

Additional evidence was provided by superimposing the predicted SPA4 peptide-binding site on a computer model exhibiting LPS-binding site within the TLR4-MD2 complex (Carpenter et al., 2009). Herein it was observed that the binding site of the SPA4 peptide to TLR4 remains farther away from the LPS-binding site (FIG. 21B).

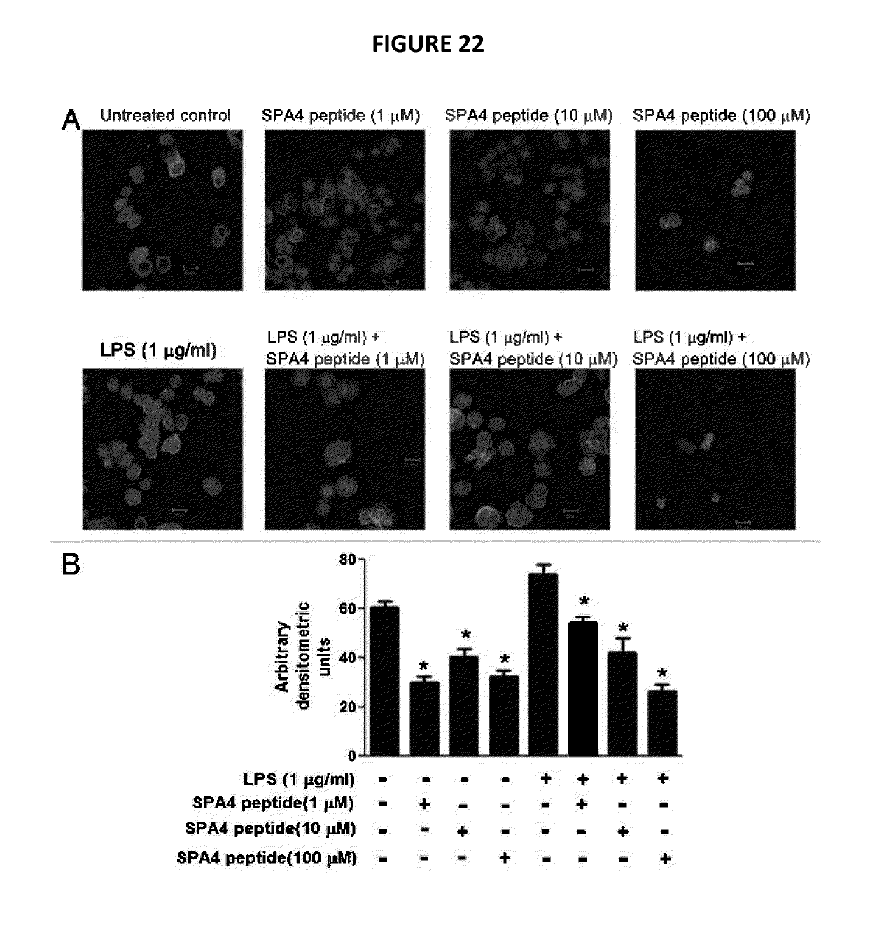

SPA4 peptide reduces the expression of TLR4. Next, the effects of SPA4 peptide on the expression of TLR4 were investigated by confocal microscopy. The results show that SW480 cells constitutively express TLR4. As expected, the TLR4 expression is further increased in response to Gram-negative bacterial LPS. However, the SPA4 peptide treatment reduced the LPS-stimulated TLR4 expression to basal level (FIG. 22), which was more pronounced with 100 μM SPA4 peptide, the maximum concentration tested.

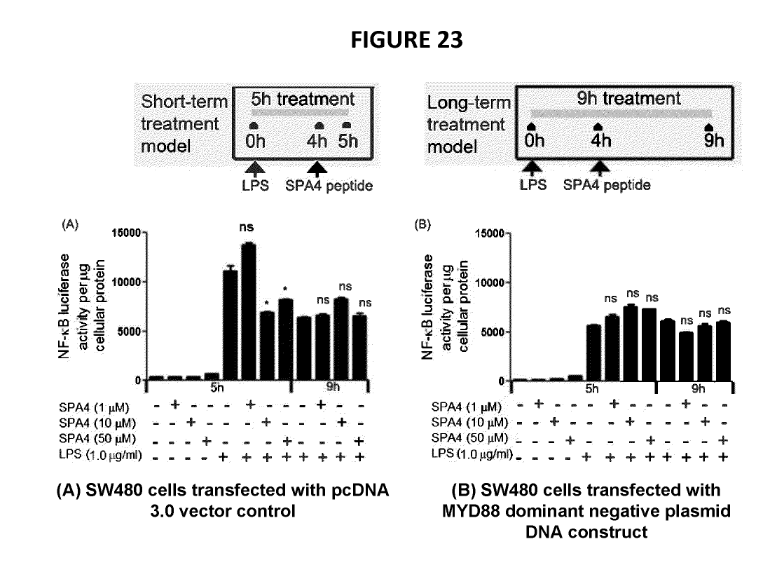

SPA4 peptide inhibits the LPS-induced MYD88-dependent NF-κB activity: Myeloid differentiation primary response gene (88; MYD88) is a known adaptor molecule of TLR4 and serves as an important molecule downstream of LPS-TLR4-MD2 binding, but upstream of activation of transcription factors (AP-1 and NF-κB) and transcription of cytokine and chemokine genes. The MYD88 engages IL-1 receptor-associated kinase (IRAK) molecule through its death domain and transduces the signal. In this Example, SW480 cells were transfected with MYD88 dominant negative construct (MYD88DN) lacking the death domain. The transfection efficiency was observed as 63-68%. The NF-κB activity was measured using a reporter plasmid (pGL4.32, Promega, WI) that contained five copies of NF-κB-response element driving the expression of luciferase reporter gene. The results show that the SPA4 peptide (at 10 and 50 μM concentrations) reduces the LPS induced-NF-κB activity after 5 hours. At 9 hours of treatment, the LPS induced NF-κB activity was, however, not significantly inhibited by 1, 10 or 50 μM concentrations of SPA4 peptide. The MYD88DN-transfected cells exhibited reduced NF-κB activity against LPS stimuli as compared to that in pcDNA3.0 vector plasmid DNA-transfected cells. The SPA4 peptide-treatment did not further reduce the NF-κB activity in cells transfected with dominant negative construct of MYD88, illustrating that the SPA4 peptide treatment affects only the MYD88-dependent NF-κB activity and does not affect the MYD88-independent NF-κB activity (FIG. 23).

Expression of NF-κB signaling molecules is affected by SPA4 peptide: The effects of SPA4 peptide on LPS-induced TLR4-NF-κB signaling were studied in cells treated with SPA4 peptide (1, 10 and 50 μM) for a short-term and a long-term basis. The expression of NF-κB-signaling molecules was measured in cell lysates by immunoblotting. The SPA4 peptide led only to subtle changes in the expression levels of NF-κB related signaling molecules, yet it caused a considerable decrease in the phosphorylation of p65. Conversely, LPS stimulated p65 phosphorylation.

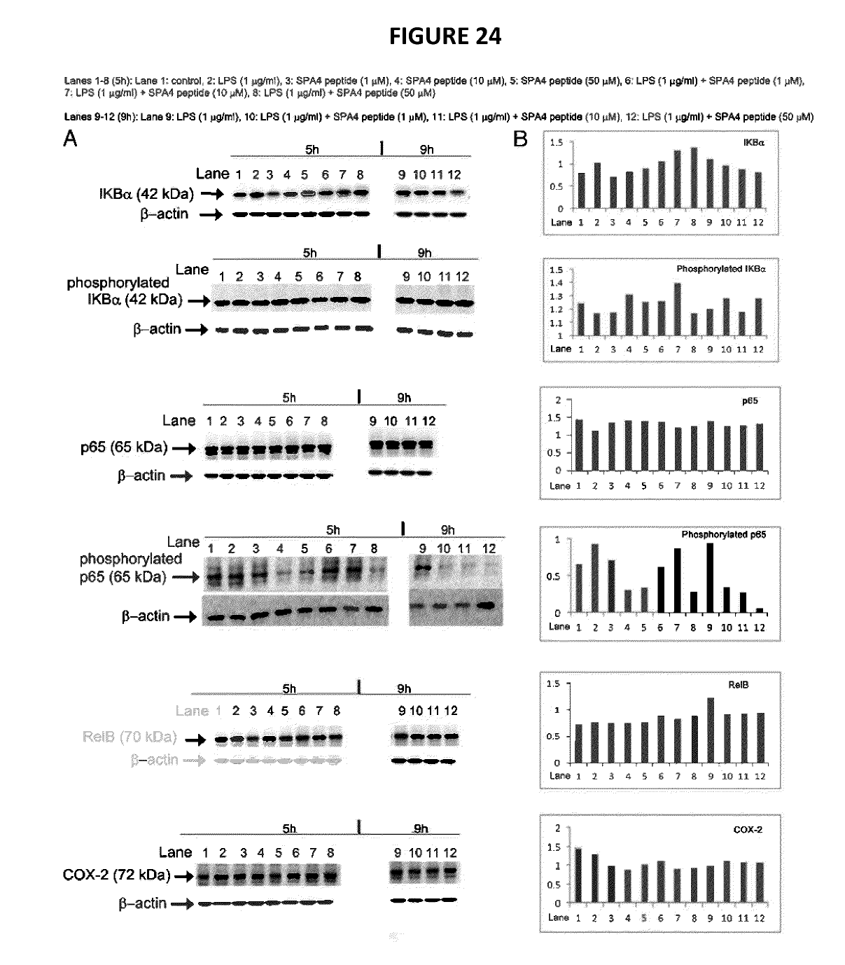

When LPS-challenged SW480 cells were treated with SPA4 peptide for 1 hour, a significant decrease was observed in the phosphorylation state of p65. This decrease was also evident when the treatment with SPA4 peptide was extended to 5 hours. The SPA4 peptide led to a modest initial increase in the expression of IKB alpha, which later reached to control level. However, SPA4 peptide did not alter, or did so only slightly, the LPS-stimulated changes in the expression or activation of other NF-kappa B related signaling molecules (FIG. 24).

SPA4 peptide inhibits the intracellular expression of IL-1β and IL-6. Immunoblotting with anti-human IL-1β antibody recognized three major immunoreactive bands (identified as numerals 1, 2 and 3 in FIG. 25) in SW480 cell lysates. IL-1β is produced as an inactive 31 kDa precursor (also known as pro IL-1β that undergoes enzymatic cleavage to a biologically active form (17.5 kDa) (identified as 2 and 3). The uppermost immunoreactive band most likely represents the IL-1β/binding protein complex. On comparison of β-actin-normalized densitometric units, the SPA4 peptide was found to inhibit the generation of the active (17.5 kDa) IL-1β for in a dose-dependent manner.

Next, the expression of IL-6 in cell-lysates of SW480 cells treated with LPS±SPA4 peptide was studied. Three major immunoreactive bands of IL-6 were detected (FIG. 25). IL-6 has been recognized to be secreted as a heterogenous set of proteins with molecular weights ranging from 19-70 kDa. Immunoreactive bands 2 and 3 represent the IL-6 dimer and monomer, respectively. The heaviest immunoreactive protein band represents the multimeric form of Il-6. Only subtle changes were observed in IL-6 expression by SW480 cells treated with LPS and the SPA4 peptide for 1 h or 5 h.

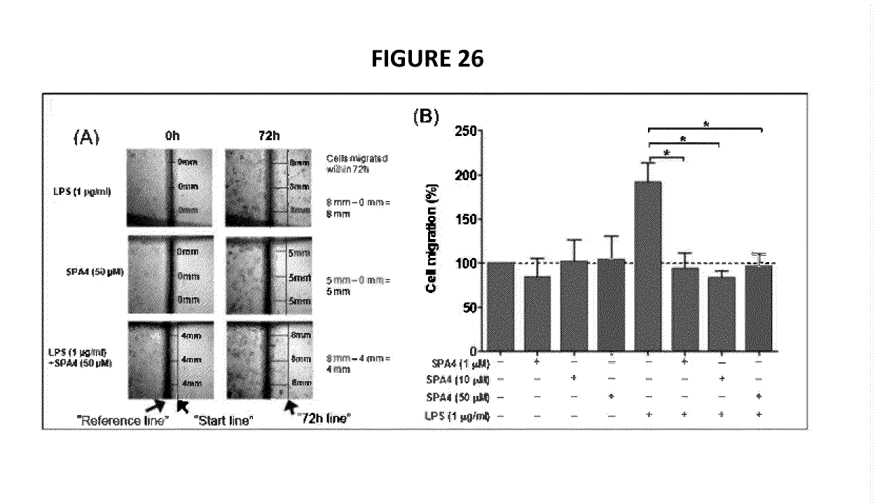

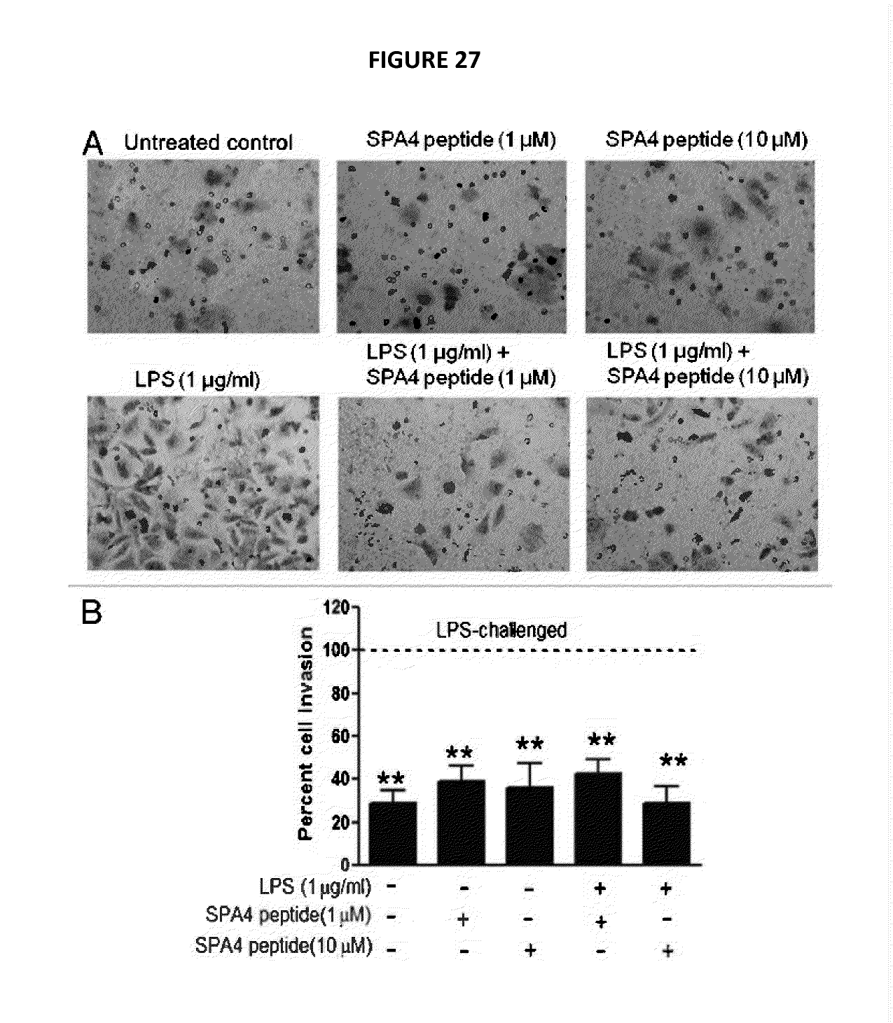

SPA4 peptide treatment inhibits the LPS-induced migration and invasion of SW480 cells. Increased cell migration and invasion are known characteristics of tumor metastasis. As expected, LPS was found to induce the metastatic properties of cell migration and invasion in SW480 cells (FIGS. 26 and 27, respectively).

Treatment with the SPA4 peptide inhibited the LPS-induced migration of SW480 cells (FIG. 26). The experiments were designed in a manner that the SPA4 peptide was added to the cells after 4 hours of LPS-challenge; the LPS and SPA4 peptide remained in the medium for the total duration of 5 hours (in FIGS. 22-25) or thereafter. The inhibitory effect of SPA4 peptide on LPS-induced cell migration was apparent as early as within 24 hours of treatment (data not shown) and remained consistent till 72 hours of treatment. Thus, it is likely that the inhibition of migration of SW480 cells is initiated early and is maintained on long term basis. Furthermore, it was found that the LPS-stimulated invasion of SW480 cells through Matrigel matrix was significantly inhibited by SPA4 peptide treatment over a period of 96 hours (p<0.001; FIG. 27). Treatment with SPA4 peptide alone, however, did not affect the invasion of SW480 cells.

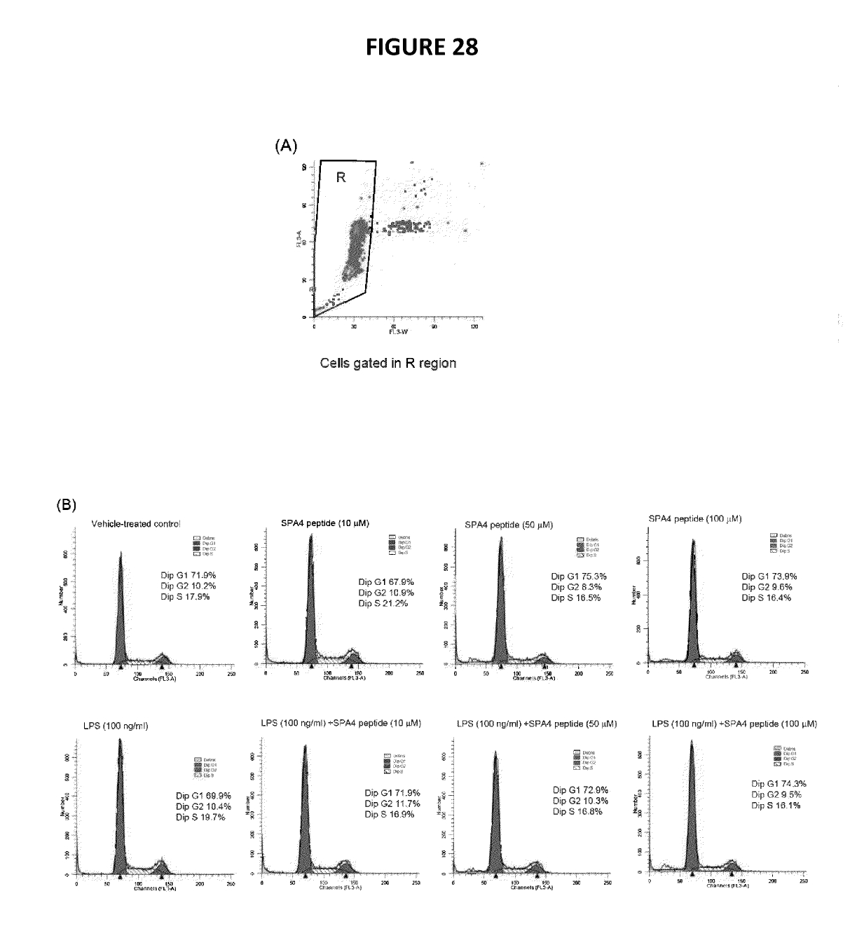

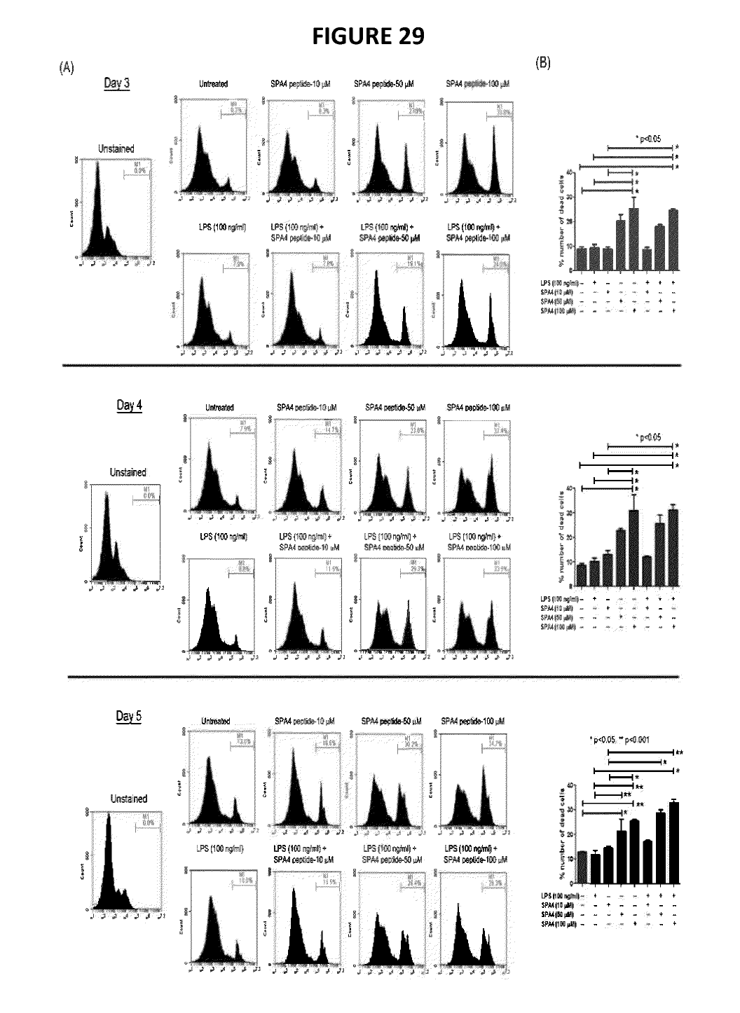

SPA4 peptide treatment does not affect the cell cycle progression, but inhibits cell viability. It was found that LPS-treatment did not affect cell cycle progression or viability. Treatment with SPA4 peptide (10, 50, and 100 μM concentrations) did not affect the cell cycle progression of SW480 cells over a period of 40 hours (FIG. 28). However, the viability of SW480 cells was reduced by SPA4 peptide as compared to untreated or LPS-stimulated cells (about 70% cells viable after SPA4 peptide treatment versus 90% cells viable after LPS- or no-stimulation; FIG. 29). The changes in cell-viability were observed as early as within three days of SPA4 peptide treatment. The inhibition of cell-viability was dependent on the concentration and duration of the treatment with the SPA4 peptide.

Discussion of Example 4:

Toll-like receptor 4 (TLR4) has been well-recognized for its critical role in sensing of pathogens or pathogen-derived signals and immune regulation. Although its involvement in cancer has not been fully-established, an increased expression of TLR4 is associated with inflammation induced cancer. Correspondingly, reduced TLR4 activity was found to inhibit inflammatory cytokine secretion, cancer cell proliferation and cancer-associated pain. On the basis of these initial findings, it is proposed herein that TLR4-blocking novel therapeutics will help reduce the inflammation and inflammation-induced cancer-progression. SW480 colorectal cells were utilized as the model system for inflammation-induced cancer progression; the SW480 cells constitutively express TLR4. This Example was focused on studying the effects of a TLR4-interacting SPA4 peptide on TLR4-signaling, inflammatory response, cell migration, cell cycle and viability of SW480 cells.

The SPA4 peptide is derived from an endogenous host-defense protein: surfactant protein-A (SPA). SPA is mainly expressed as a component of lung surfactant; its expression has been noted at other mucosal surfaces, such as intestine, skin, eye, and urinogenitary systems. In lung, SPA maintains normal lung function and exerts anti-microbial and anti-inflammatory effects against pathogens and stress-ligands. Since during infection and inflammation, the amounts of SPA are reduced significantly, an SPA based surfactant or therapeutic may be of clinical value. It has not been possible to formulate an SPA-based therapeutic because of its large size and its amenability to degradation. Using computer-modeling and functional screening of a small peptide library, a shorter region of SPA was identified from the TLR4-interacting region and referred to herein as the SPA4 peptide. Example 2 illustrates that the SPA4 peptide inhibits the LPS-induced inflammatory response in JAWS II dendritic cell line.

TLR4-signaling is induced in response to a number of endogenous and exogenous ligands, including bacterial LPS. LPS binds to TLR4-MD2 complex and induces inflammatory response via activation of a complex intracellular signaling network. The SW480 cells constitutively express TLR4, and LPS is known to induce inflammatory response, adhesion and migration. Thus, this system was utilized to investigate the effects of SPA4 peptide on LPS-induced TLR4-NF-κB signaling, inflammation and cancer cell properties. In order to understand the mechanism of action of SPA4 peptide, first it was confirmed that the SPA4 peptide does not directly bind to stress-ligand LPS, and the binding site of LPS onto the TLR4-MD2 complex is farther away from the binding site of SPA4 peptide. (FIG. 21). These results indicate that the SPA4 peptide neither binds to LPS nor interferes with the binding of LPS to TLR4-MD2 complex. Furthermore, since SPA4 peptide is hydrophilic (Ko/w=0.56), the SPA4 peptide most likely does not enter into the cytoplasm of the cell by crossing the hydrophobic cell membrane, and does not directly affect the intracellular inflammatory signaling. Overall, these results support the notion that the anti-inflammatory effects of SPA4 peptide are exhibited through its interaction with TLR4.

LPS is known to induce inflammation via activation of TLR4-NF-κB signaling in MYD88-dependent as well as MYD88-independent manner. Full-length SPA has been identified to inhibit NF-κB activity, but upstream molecular events occurring after SPA-TLR4 interaction remain unknown. As such, the LPS-TLR4-MD2-signaling is quite complex. Nothing is known in particular for SPA4 peptide. This Example shows that the SPA4 peptide inhibits NF-κB-activity in a MYD88-dependent manner (FIG. 23). Furthermore, SPA4 peptide induces the expression of IKBα, an inhibitor of NF-κB, but does not affect the Ser 32-phosphorylated IKBα. The increase in the expression of IKBα is in accordance with the published data on full-length SPA (Wu et al., 2004). Since NF-κB is composed of five different subunits, c-Rel, RelB, p65, p50 and p52, it was investigated if the stimulation of IKBα translates into the inhibition of these downstream factors. Inhibition of phosphorylation of p65 by SPA4 peptide translates into the inhibition of NF-κB activity (FIG. 24). These results illustrate that SPA4 peptide inhibits the TLR4-NF-κB signaling in response to inflammatory stimuli.

An oscillating pattern of expression of signaling molecules were observed in 5 hour and 9 hour treatment-models; it is possible that different mechanisms in conjunction with NF-κB are affected downstream of the binding of SPA4 peptide to TLR4. In conjunction with the inhibition of NF-κB signaling by SPA4 peptide, a significant decrease in the amounts of LPS-induced IL-1β and IL-6 cytokines were observed. This reduction in inflammatory signaling and cytokine response correlated with inhibition of LPS-induced cell migration. Although a change in cell cycle progression was not observed, significant reduction in cell viability was observed upon treatment with SPA4 peptide. The percent cell-death increased as per the increase in duration and treatment dose of SPA4 peptide. These results will facilitate development of a novel chemopreventive immunotherapeutic to control inflammation-induced cancer progression in patients with colitis.

### Example 5

### Inhibition of LPS-Induced Acute Inflammation by SPA4

In Example 5, the efficacy of SPA4 peptide in inhibiting LPS-induced acute inflammation was investigated in a mouse model of LPS-inflammation. Balb/c Mice were challenged with E. coli 0111:B4 highly purified, low protein-derived LPS (0.1 or 1 or 15 μg/g body wt) via an intraperitoneal route, one hour prior to treatment with SPA4 peptide (2.5 μg/g body weight) or full-length purified SPA (0.5 μg/g body weight) via an intraperitoneal route. The mice were monitored for endotoxic-shock like clinical symptoms (including, but not limited to, ruffled fur, eye exudates, diarrhea, prostration, and lack of reactivity), and these symptoms were scored on the basis of severity from 0 to 3, and average clinical symptom indices were calculated for each animal based on the symptom grades (Metkar et al. (2007) Infect Immun, 75:5415-5424). Endotoxic-shock like clinical symptoms were noted (at 5-7 hours), and mice were sacrificed at different time-intervals after LPS challenge. Major organs and blood specimens were collected. Secreted amounts of TNF-α cytokine were measured in serum and lung tissue homogenates by ELISA method.

At the molecular level, the effects of SPA4 peptide on LPS-induced NF-κB activity were studied in dendritic cells (JAWS II cells) co-transfected with NFκB luciferase reporter plasmid construct and dominant negative construct of MYD88 (an adaptor protein downstream of TLR4; MYD88DN). Briefly, JAWS II dendritic cells (0.5×10⁶) were transfected with NF-κB luciferase reporter and/or MYD88DN using TranslT-TKO transfection reagent as described earlier (Awasthi et al. (2003) Biotechniques, 35:600-602, 604). After 20-24 hours of incubation, the transfected cells were challenged with LPS (100 ng/ml) for 4 hours and treated with SPA4 peptide (10 μM) for 1 hour. After the completion of 5 hours incubation, the cell lysates were prepared in cell lysis buffer and NF-κB-luciferase reporter activity was measured as per the manufacturer's instructions (Promega, WI).

SPA4 peptide inhibits LPS-induced NF-κB activity upstream of MYD88 in dendritic cells. The MYD88 is an intracellular adaptor protein which transduces the LPS-TLR4-signal to NF-κB. Although LPS-TLR4 binding is known to induce NF-κB in MYD88-independent manner also, the inhibition of the NF-κB activity in MYD88-/-transfected dendritic cells by SPA4 peptide was equivalent to that in untreated MYD88-/-transfected dendritic cells against LPS challenge (FIG. 37). These results illustrate that SPA4 peptide does not affect MYD88-independent NF-κB activity. These results are in accordance with the results presented in SW480 cells in Example 4 (FIG. 23).

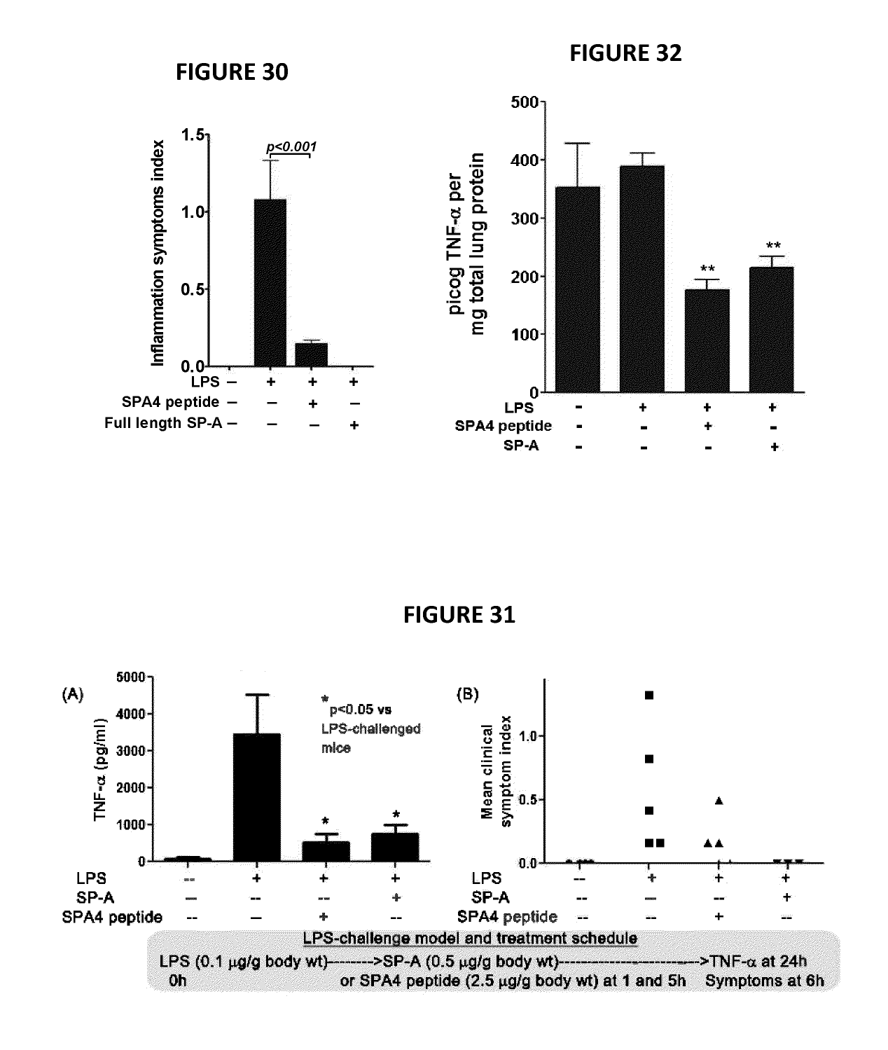

The endotoxic shock-like symptoms induced after LPS challenge were measured (Metkar et al., 2007). Results presented in FIG. 30 show that SPA4 peptide and full-length SPA inhibit the LPS (0.1 μg/g body weight)-induced clinical symptoms.

LPS is known to induce the secretion of TNF-α, and a significant amount is detected in the blood. In FIG. 31, it was found that LPS (0.1 μg/g body weight)-induced circulatory TNF-α amounts are reduced after treatment with SPA4 peptide and full-length SPA. The results are in accordance with published data in the literature on anti-inflammatory effects of full-length SPA. Similar to SPA, SPA4 peptide also inhibited LPS-induced TNF-α in vivo through its interaction with TLR4 and suppression of NF-κB activity. Also, these results corroborate with findings presented in Examples 2 and 3.

SPA4 peptide treatment also reduced LPS-induced TNF-α levels in lung tissue homogenates. FIG. 32 illustrates that the amounts of LPS-induced TNF-α are low in lung tissues of mice challenged with LPS (1 μg/g body weight) and treated with full-length SPA and SPA4 peptide. Similar observations are expected for SPA4 peptide inhibition of the inflammatory response in other organs.

In summary, the results show that the treatment with SPA4 peptide inhibited NF-κB luciferase activity in dendritic cells, inflammatory symptoms, and TNFα levels in serum and lung tissue homogenates of mice against LPS stimuli. Moreover, the inhibition of LPS-induced NF-κB activity by SPA4 peptide was indistinguishable from those challenged with LPS alone, in absence of functional MYD88. Taken together, the results indicate that the SPA4 peptide exerts its inhibitory effect on the TLR-NF-κB pathway upstream of MYD88 adaptor protein and thereby regulates the TNFα response.

### Example 6

### SPA4 Peptide Inhibits Lipopolysaccharide-Induced Lung Inflammation

An uncontrolled inflammatory response against infectious stimuli can lead to severe damage or failure of important organs. Endotoxic shock-induced acute respiratory distress syndrome (ARDS) and multiple organ failure represent this condition. Conventional corticosteroids and selective blockade of isolated aspects of inflammatory state are practiced; none of them have been proven completely beneficial for the treatment of ARDS. Corticosteroids are known to affect functions of many cells and systems within the body including the suppression of the immune system. Others target the preformed already-secreted cytokines and chemical mediators, for example: anti-TNF-α, IL-1β and IL-6-antibodies and receptor antagonists. These agents provide only a transient relief and pose significant side-effects including the serious risk of secondary infections. Therapeutic inhibition of inflammatory signaling may provide better results and help alleviate the clinical symptoms.

Toll-like receptor-4 (TLR4) recognizes pathogen-associated molecular patterns derived from pathogens, including the lipopolysaccharide (LPS) of Gram-negative bacteria. It also recognizes endogenous damage-associated molecular patterns, for example: fibronectin, heat-shock proteins, released as a consequence of an inflammatory response. Upon binding to the ligand, TLR4 induces a complex intracellular signaling through its Toll/interleukin-1 receptor domain and transcription factors, including nuclear factor (NF)-κB, which leads to synthesis of cytokines/chemokines and other inflammatory mediators. Surfactant protein-A (SPA) expressed by epithelial cells at various mucosal surfaces in our body is recognized as the secretory pathogen-recognition receptor. The inventor and others have shown that the SPA interacts with multiple receptors, including TLR4. Through interaction with TLR4, the SPA modulates host defense functions of immune cells against infectious stimuli: SPA reduces the release of TLR4-stimulated pro-inflammatory TNF-α cytokine, but preserves the TLR4-induced phagocytosis. Thus, a TLR4-interacting region of SPA may mimic host defense functions of SPA. A 20-mer SPA4 peptide with the amino acid sequence GDFRYSDGTPVNYTNWYRGE (SEQ ID NO:3) has recently been identified that interacts with TLR4 and inhibits the LPS-stimulated release of TNF-α in an immortalized dendritic cell line (see Example 2); the underlined amino acid sequences of SPA4 peptide were observed to be in close proximity of TLR4 in an in silico model of SPA-TLR4-MD2 protein complex. In this study, the SPA4 peptide was assessed for its ability to suppress the LPS-TLR4-stimulated inflammation via its interaction with TLR4 and help to alleviate the inflammatory parameters and clinical symptoms in a mouse model.

The results of this Example demonstrate that the SPA4 peptide region contributes to SPA-TLR4 interaction, inhibits the LPS-induced inflammatory parameters (TNF-α secretion, NF-κB activity, influx of cells), and alleviates the endotoxic shock-like symptoms in a mouse model of LPS-induced inflammation.

Materials and Methods of Example 6:

Animals: The animal studies were approved by the Institutional Animal Care and Use and Institutional Biosafety Committees at the University of Oklahoma Health Sciences Center (OUHSC), Oklahoma City, Okla. BALB/c mice (female, 5-6 weeks old) were included and housed for one week for acclimatization at the College of Pharmacy Animal Facility, OUHSC, Oklahoma City, Okla., prior to conducting any experiment.

Protein-protein interaction by mammalian two-hybrid assay: Interaction between (i) SPA and TLR4, and (ii) an SPA-mutant lacking SPA4 peptide region and TLR4, was assessed in HEK293 cells by a mammalian two-hybrid assay (Promega, WI). The functional relevance of the SPA-TLR4 interaction was simultaneously studied by measuring NF-κB activity in this system.

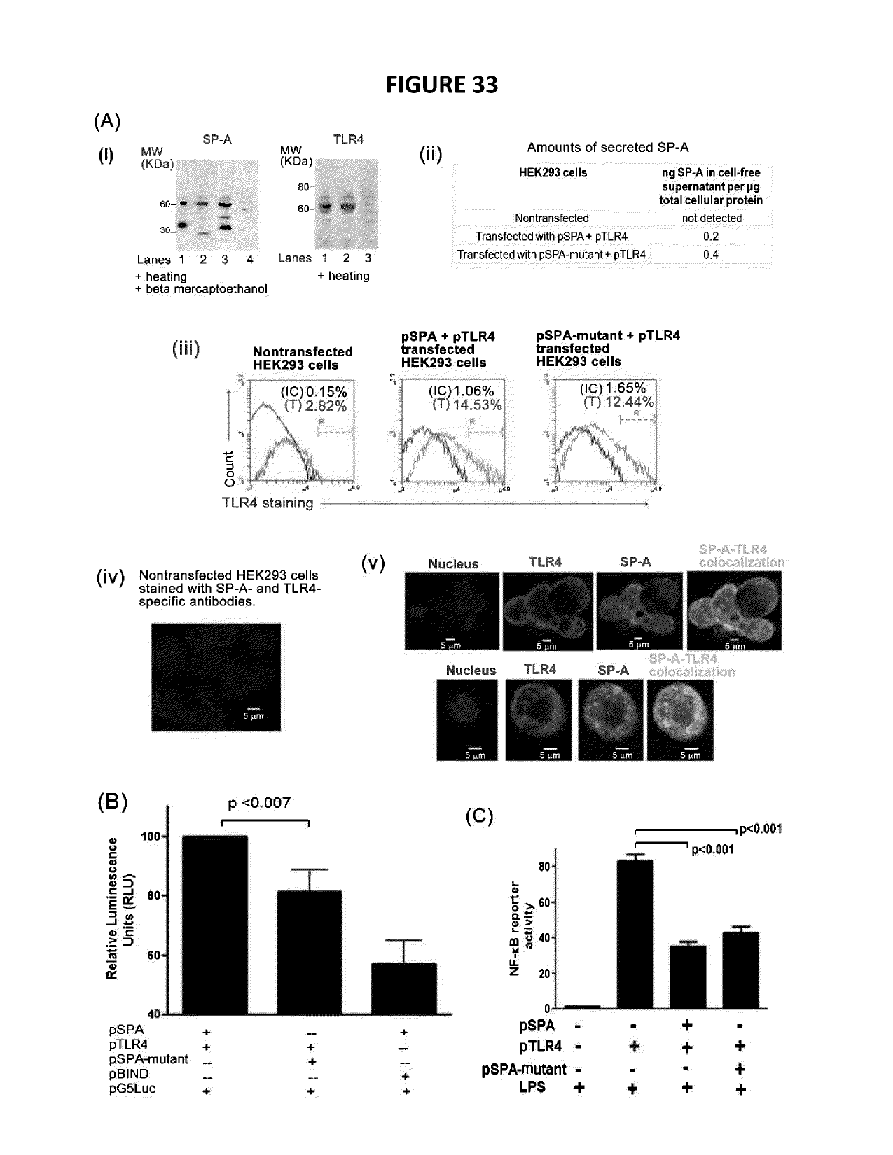

HEK293 cell culture: Human embryonic kidney epithelial cells (HEK293 obtained from Dr. Kelly Standifer, Department of Pharmaceutical Sciences, OUHSC, Oklahoma City) were included to study the protein-protein interaction because HEK293 cells do not express endogenous SPA or TLR4 (FIG. 33). The HEK293 cells were maintained in Dulbecco's modified Eagle medium (DMEM) containing 0.37% sodium bicarbonate, 5% heat-inactivated fetal bovine serum (FBS), 100 U/ml penicillin and 100 μg/ml streptomycin antibiotics (Invitrogen, NY).

Plasmid DNA constructs: Plasmid DNA constructs encoding full-length human SPA (pSPA), SPA-mutant lacking SPA4 peptide region (pSPA-mutant) and full-length human TLR4 (pTLR4) were prepared using recombinant DNA methods (Mutagenex, NJ). The SPA- and TLR4-cDNA inserts were obtained from pCR-BluntlI-TOPO (Open biosystems, CO) and pUNO1-hTLR04a (Invivogen, CA) plasmid DNAs, respectively. SalI and MluI restriction sites were added to the SPA-cDNA fragment and cloned into the pACT plasmid DNA to obtain pSPA (Promega, WI). Subsequently, deletion mutant of SPA lacking SPA4 peptide region (pSPA-mutant) was prepared by site-directed mutagenesis. The pTLR4 construct was prepared by PCR subcloning of the TLR4-cDNA insert into the pBIND plasmid DNA backbone at the BamHI-MluI sites (Promega, WI). The pSPA and pSPA-mutant plasmid DNA constructs were designed to encode SPA and SPA-mutant proteins as fusion proteins with VP16, and pTLR4 plasmid construct to encode TLR4 as fusion protein with GAL4, respectively. The cloning direction, reading frame and insert size within the plasmid constructs were confirmed by DNA sequencing and restriction digestion analysis. The plasmid DNAs were prepared using endotoxin-free plasmid DNA extraction kit (Qiagen, CA), and endotoxin content was checked by Limulus amebocyte lysate (LAL) assay kit (Charlesriver Lab, MA).

Mammalian two-hybrid assay: The cells were transfected with pSPA or pSPA-mutant, pTLR4 and pG5Luc (encoding Firefly luciferase) plasmid DNAs. Transfection conditions were optimized using different cell numbers and plasmid DNAs to Lipofectamine 2000 transfection reagent (Invitrogen, CA) ratios. In the comprehensive experiments, HEK293 cells were seeded at the density of 2.5×10⁵ cells per ml and transfected using 0.2 μg plasmid DNA each per 1 μl Lipofectamine 2000 transfection reagent. After 24 hours of transfection, the cells were washed and scraped in 20 μl lysis buffer provided with the Dual luciferase reporter assay kit (Promega, WI). Firefly and Renilla luciferase-associated luminescence were read using the Synergy HT multi-mode microplate reader (Biotek, VT). The lysates of (i) nontransfected cells, and cells transfected with (ii) pACT and pBIND vector plasmid DNAs, and (iii) pSPA and pBIND (vector backbone for pTLR4), served as negative controls. Plasmid DNAs: pACT-MyoD and pBIND-Id, provided with the kit, served as assay controls.

The following calculations were performed after obtaining the raw data for Firefly and Renilla luciferase. The Firefly luciferase activity-associated luminescence value for each cell lysate was divided by its Renilla luciferase activity-associated luminescence value and multiplied by 1000. The luciferase activity was then expressed in relative luminescence units (RLU). In all the experiments, the RLU for cells transfected with pSPA and pTLR4 was set at 100.

Immunoblotting for SPA and TLR4: The cells were homogenized in a homogenization buffer containing a cocktail of protease inhibitors (1 mM ethylenediaminetetraacetic acid; EDTA, 1.1 μM leupeptin, 1 μM pepstatin, 0.2 mM phenylmethyl sulphonyl fluoride; PMSF) and detergents (0.1% sodium dodecyl sulfate; SDS, 1% Igepal CA630; Awasthi et al. (2001) Am J Respir Crit Care Med. 163(2):389-397). Total protein concentration was measured in lung tissue homogenates by bicinchoninic acid (BCA) protein assay kit. The total cell lysate proteins were separated on Novex 4-20% tris-glycine SDS-PAGE gradient gel (Invitrogen, CA) and transferred onto nitrocellulose membrane. The non-specific sites were blocked using 7% non-fat milk solution. The membrane was then incubated with 1:1,000 diluted anti-human SPA or 1:500 diluted anti-human TLR4 (Abcam, MA) antibodies. The membrane was washed and incubated further with 1:1,000 diluted anti-rabbit horse radish peroxidase-conjugated antibody for 45 minutes. The immune complexes were visualized using the chemiluminescent substrate reagent (Pierce, IL). Immunoblots were imaged using the Ultraquant Acquisition program (Ultralum Inc, CA). Purified human lung SPA (provided by Dr. Jo Rae Wright, Department of Cell Biology, Duke University Medical Center, Durham, N.C.) was included as positive control for SPA.

Enzyme linked immunosorbent assay (ELISA) for SPA: The secreted levels of SPA were measured in freeze-concentrated cell-free supernatants by ELISA as per the method described earlier (Awasthi et al. (1999) Am J Respir Crit Care Med. 160(3):942-949). Briefly, the cell-free supernatants diluted in 0.1 M NaHCO₃ buffer at pH 9.6, were incubated overnight in multiwell Immulon strips. The nonspecific sites were blocked, and 1:1,000 diluted rabbit anti-human SPA specific antibody was added to the wells. After washing the wells, the immune complexes were incubated with 1:1,000 diluted secondary anti-rabbit horse radish peroxidase-conjugated antibody following the incubation with 75 μl of 3, 3′, 5, 5′ tetramethylbenzidine substrate solution. The reaction was stopped using 0.2 NH₂SO₄. The optical density was read spectrophotometrically. Diluted amounts of purified human lung SPA were used to prepare the standard curve. Measured amounts of SPA were normalized with total cellular protein.

Immunocytochemistry: To confirm the expression and co-localization of SPA and TLR4 proteins in transfected cells, the immunocytochemistry was performed in 8 well chamber slides (Nunc, NY). Nonspecific sites were blocked with 1% bovine serum albumin and cells were incubated with 1:500 diluted anti-human SPA (Chemicon, MA) and 200 μg/ml anti-human TLR4 (Imgenex, CA) antibodies overnight at 4° C. Subsequently, cells were washed and incubated with Alexa fluor 488-conjugated anti-rabbit antibody for SPA and Alexa fluor 568-conjugated anti-mouse antibody (10 μg/ml, both antibodies were from Invitrogen, CA) for TLR4. Finally, Hoechst dye (1 μg/ml) was added for nuclear staining and the slides were mounted with Vectashield (Vector Laboratories Inc, CA). All the images were acquired at 63× oil immersion objective under Zeiss confocal microscope and processed using Zeiss LSM Image Examiner or ZEN 2011 programs.

Flow cytometry: The cell-surface expression of TLR4 was also investigated by flow cytometric analysis of cells transfected with pSPA or pSPA-mutant, pTLR4 and pG5Luc plasmid DNAs. After 24 hours of transfection, cells were washed two times with ice-cold Dulbecco's phosphate buffered saline (DPBS), stained with phycoerythrinin (PE)-conjugated anti-TLR4 antibody (clone HTA125; eBioscience, CA) for 45 minutes and fixed in 0.5% paraformaldehyde solution. Finally, the fixed cells were run on Accuri flow cytometer (BD Biosciences, CA), and TLR4-staining was analyzed using C6 software.

NF-κB reporter activity assay: The functional relevance of interaction of SPA or SPA-mutant with TLR4 was assessed in the two-hybrid HEK293 cell system by NF-κB reporter activity assay. Briefly, HEK293 cells (50,000 per well) were seeded in a 96-well tissue-culture plate (BD Falcon, NJ). The cells were transfected with 0.2 μg plasmid DNA each of pSPA or pSPA-mutant, pTLR4 and pGL4.32 NF-κB reporter plasmid DNA (luc2P/NF-κB-RE/Hygro, Promega, WI) using 1 μl Lipofectamine 2000 reagent per well. After 24 hours of transfection, cells were washed once with plain DMEM and treated with highly-purified, low lipoprotein Escherichia coli O111:B4 LPS (100 ng/ml; Calbiochem, CA) for 5 hours. After completion of incubation, cells were washed once with ice-cold DPBS and scraped in 20 μl of cell lysis buffer provided with the Dual luciferase reporter assay kit (Promega, WI). Firefly and Renilla luciferase readings were recorded using the Synergy HT multi-mode microplate reader (Biotek, VT). Renilla luciferase activity provided a measurement of transfection efficiency. The luminescence units obtained for NF-κB-associated Firefly luciferase activity were normalized with those readings for Renilla luciferase. The pTLR4-transfected cells challenged with LPS alone served as control.

Synthetic SPA4 peptide: After assessing the role of SPA4 peptide region in SPA-TLR4-interaction and function in HEK293 two-hybrid assay system, the 20-mer SPA4 peptide (amino acid sequence: GDFRYSDGTPVNYTNWYRGE (SEQ ID NO:3)) was included to investigate its direct anti-inflammatory effects in a dendritic cell line and in a mouse model. The SPA4 peptide was synthesized by Genscript, NJ, and purity was confirmed by mass-spectroscopy and high-performance liquid chromatography. Endotoxin content in SPA4 peptide suspensions was measured by LAL assay. Predictions about primary structure, physico-chemical features and three-dimensional (3D) confirmation were obtained using PepDraw (Tulane University, New Orleans, La., USA; White and Wimley (1998) Biochim Biophys Acta. 1376(3):339-352) and PEP-FOLD programs (Maupetit et al. (2009) Nucleic Acids Res. 37 (Web Server issue):W498-W503), respectively. A Kyte and Doolittle hydropathy plot was drawn to determine the hydrophobicity/hydrophilicity of the SPA4 peptide (J Mol Biol. (1982) 157(1):105-132).

SPA4 peptide activity at cellular level: As HEK293 cells do not represent the immune antigen-presenting cells, the biological effects of synthetic SPA4 peptide were studied in a dendritic cell system.

JAWS II dendritic cell culture: The JAWS II dendritic cells (ATCC, VA) derived from bone marrow of C57BL/6 mice were maintained in Alpha-modified minimum essential medium (α-MEM; Sigma, Mo.) supplemented with 20% FBS, 4 mM L-glutamine, 100 U/ml penicillin, 100 μg/ml streptomycin, 50 μg/ml gentamicin (Invitrogen, NY) and 5 ng/ml of recombinant murine granulocyte macrophage-colony stimulating factor (Peprotech, NJ) (Awasthi et al. (2005) J Immunol. 175(6):3900-3906).

Expression of phospho-NF-κB-p65: The JAWS II dendritic cells (1×10⁶ cells) were challenged with highly-purified, low lipoprotein Escherichia coli O111:B4 LPS (100 ng/ml; Calbiochem, CA) for 4 hours and subsequently treated with SPA4 peptide (1 and 10 μM) for a period of 1 hour. The cell lysates were prepared in 200 μl homogenization buffer containing protease inhibitors, detergents (as described above) and phosphatase inhibitors (0.25 mM sodium orthovandate and 500 mM sodium fluoride). The cell lysate proteins were separated on 4-20% tris-glycine SDS-PAGE gel under complete reducing condition. The expression of phosphoryated-NF-κB-p65 (Ser 276) was investigated by immunoblotting with 1:500 diluted anti-phospho-NF-κB-p65 antibody (Santacruz Biotechnology, CA) and 1:2,000 diluted anti-rabbit horse radish peroxidase-conjugated secondary antibody, as per the method described above. The membrane was stripped off the probing antibodies at 60° C. for 45 minutes using a stripping solution containing 10% SDS, 0.5 M tris and β-mercaptoethanol (35 μl per ml) and reprobed with 1:1,000 diluted anti-β-actin antibody (Biolegend, CA) to confirm equal loading. Immunoblots were imaged using the Ultraquant Acquisition program (Ultralum Inc, CA), and densitometric analysis of immunoreactive bands was performed with Image J 1.42q program. Finally, the arbitrary densitometric units for the phospho-NF-κB-p65 were normalized with those for β-actin.

NF-κB activity assay: The JAWS II cells (1×10⁶ cells) were co-transfected with pcDNA3.0 vector (obtained from Dr. Brian Ceresa, Department of Cell Biology, OUHSC) or myeloid differentiation primary response gene (MYD88)-dominant negative (MYD88DN) lacking the death and intermediate domains (obtained from Dr. Ruslan Medzhitov, Yale University, CT) and pGL4.32 NF-κB reporter plasmid DNA (luc2P/NF-κB-RE/Hygro, Promega, WI) plasmid DNA constructs (1 μg DNA each) using TranslT-TKO transfection reagent (Mirus, WI).(21, 22) The ratio of transfection reagent to DNA (2 μl per 1 μg of DNA) was kept constant. After 4 hours of incubation, cells were supplemented with an additional 250 μl of α-MEM medium containing 20% plain FBS and incubated for an additional 14-16 hours. Cells were then washed and treated with highly-purified, low lipoprotein Escherichia coli O111:B4 LPS (100 ng/ml; Calbiochem, CA) for 4 hours and subsequently treated with SPA4 peptide (1 and 10 μM) for a period of 1 hour. Luminescence of the NF-κB-associated Firefly luciferase activity was read using the Synergy HT multi-mode microplate reader as described above. Total cellular protein content was estimated using a BCA protein assay kit (Pierce, IL), and was used to normalize the NF-κB-associated luciferase activity.

Cytokine (TNF-α) measurement: The TNF-α levels were measured in cell-free supernatants of JAWS II cells treated with LPS+SPA4 peptide by enzyme linked immunosorbent assay (ELISA) (Vilekar et al. (2012) Int Immunol. 24(7):455-464). The secreted levels of TNF-α were normalized with total cellular protein.

Mouse model of LPS-induced lung inflammation: A mouse model of LPS-induced lung inflammation was included to assess the in vivo efficacy of SPA4 peptide. In the pilot experiments, mice were injected with 1.0, 10, 15, and 20 μg of LPS per g body weight through the intraperitoneal route, and observed for endotoxic shock-like symptoms and histological evidence of influx of leukocytes. The dose of 15 μg LPS/g body weight was found optimum to induce endotoxic shock-like symptoms and severe inflammation in lung without causing any mortality within the study-period. Thus, for the comprehensive experiments, the mice were challenged with 15 μg LPS/g body weight. Control mice received an equal volume of endotoxin-free saline. After 1 hour of LPS challenge, mice were injected with SPA4 peptide: 2.5 μg/g body weight or purified lung SPA: 0.5 μg/g body weight (provided by Dr. Jo Rae Wright, Department of Cell Biology, Duke University Medical Center, Durham, N.C.) via the intraperitoneal route. The treatment dose of SPA4 peptide was kept 5 times higher as compared to that of purified lung SPA, because of the difference in their binding affinity to TLR4 (Awasthi et al. (2011) J Pharmacol Exp Ther. 336(3):672-681).

Mice were monitored for signs of endotoxic shock-like symptoms. After 6 hours of LPS-challenge, the endotoxic shock-like symptoms (ruffled fur, eye exudates, prostration, signs of diarrhea and lack of reactivity) were noted for each mouse on the scale of 0-3. An average symptom index score was obtained for each mouse in a group (Metkar et al. (2007) Infect Immun. 75(11):5415-5424).

Subsequently, mice were anaesthetized and euthanized. A sample of blood was collected via cardiac puncture, centrifuged to obtain serum, aliquoted and stored at −80° C. Major organs were harvested under aseptic conditions. Lung tissues were either snap-frozen in liquid nitrogen or fixed in 10% buffered formalin. At the time of analyzing inflammatory parameters, frozen lung tissues were thawed and homogenized in a homogenization buffer containing a cocktail of protease inhibitors and detergents as described earlier (Awasthi et al. (2001) Am J Respir Crit Care Med. 163(2):389-397). Total protein concentration was measured in lung tissue homogenates by BCA protein assay kit.

Tissue histopathology: After fixing overnight in 10% buffered formalin, the lung tissue specimens were transferred to 75% ethanol. Tissues were processed to further dehydrate, clear and infiltrate into 70%-100% alcohol and xylene, and embedded into paraffin. Lung sections (5 μm in thickness) were obtained and stained with hematoxylin and eosin (H&E).

Measurement of cytokine (TNF-α): The TNF-α levels were measured in diluted serum samples and lung tissue homogenates by ELISA (Awasthi et al. (2003) Biotechniques. 35(3):600-602, 604). The amounts of TNF-α measured in lung homogenates were normalized with total protein.

Levels of myeloperoxidase (MPO) in lung tissue homogenates: Myeloperoxidase (MPO, EC 1.11.1.7) is a lysosomal hemeprotein located in the azurophilic granules of neutrophils and monocytes. Increased MPO expression is associated with inflammation.(25) Thus, the MPO levels were measured in lung homogenates using a commercially-available ELISA kit (Invitrogen-Molecular Probes, CA). Briefly, plate wells were coated with 500 ng/ml mouse anti-MPO antibody for 1 hour at room temperature. After washing the wells, MPO standard solutions (0.75-100 ng/ml) and diluted lung homogenates were added and incubated for 1 hour at room temperature. The antigen-antibody immune complexes were then incubated with 1 μg/ml rabbit anti-MPO secondary capture antibody and 100 ng/ml goat anti-rabbit horse radish peroxidase labeled (HRP)-IgG. Finally, the Amplex UltraRed reagent, a fluorogenic substrate for HRP, was added and fluorescence was read at the setting of 530 nm (excitation) and 590 nm (emission) wavelengths on the Synergy HT multi-mode microplate reader.

Lung immunohistochemistry for NF-κB-p65: Five μm sections of lung were deparaffinized in xylene and rehydrated through a graded ethanol series. Antigen retrieval was performed in pH 6.0 citrate buffer prior to staining. Non-specific binding sites were blocked with normal mouse serum. The tissue sections were then incubated overnight with NF-κB-p65 antibody (1:5,000 dilution, Santa Cruz Biotechnology, CA). Finally, NF-κB-p65 localization was detected using Vectastain ABC anti-rabbit IgG kit and Vector Blue alkaline phosphatase substrate system (Vector Laboratories, CA). The lung sections were counterstained with nuclear fast red, cleared with a xylene substitute, and coverslips were permanently mounted using non-xylene based mounting medium. The slides were examined for NF-κB-p65 expression and nuclear localization under light microscope.

Statistical analysis: The results were analyzed for statistical significance by Student t-test or ANOVA using Prism software (Graphpad, CA). The p values at <0.05 were considered significant or otherwise noted.

Results for Example 6:

As shown earlier Examples, the SPA4 peptide region was identified from the TLR4-interacting site in an in silico model of SPA-TLR4-MD2 complex, and direct binding of the synthetic SPA4 peptide to TLR4 was studied using an in vitro microwell-binding assay. Relevance of the SPA4 peptide region in SPA-TLR4-interaction remained unknown in a cellular system. In this Example, the mammalian two-hybrid assay was utilized to assess the contribution of the SPA4 peptide region in the SPA-TLR4 interaction in HEK293 cells which do not express endogenous SPA and TLR4 and provide a cleaner system for analysis of SPA-TLR4 interaction. The two-hybrid system is based on the modular domains found in some transcription factors: a DNA-binding (DB)-domain, which binds to a specific DNA sequence, and a transcriptional activation (TA)-domain, which interacts with the basal transcriptional machinery. A TA-domain in association with a DB-domain promotes the assembly of RNA polymerase II complexes at the TATA box and increases transcription. The DB-(GAL4) and TA-(VP16) domains are produced by separate plasmids. The two-hybrid assay has been utilized for studying interaction between various protein-partners. Similarly, the transcription machinery was expected to be activated by interaction between TLR4 fused to a GAL-4 DB-domain and SPA protein fused to a VP16 TA-domain. The SPA-TLR4 interaction would then result in the transcription of Firefly luciferase reporter gene.

HEK293 cells were utilized because the HEK293 cells did not express endogenous SPA or TLR4 proteins as confirmed by real-time PCR (results not shown), western blotting, flow cytometry, and immunocytochemistry (FIG. 35A). All the experiments were performed in endotoxin-free conditions; the plasmid DNA suspensions had <0.000285 ng endotoxin per μg DNA as detected by LAL assay kit. Under optimized experimental conditions, about 70% transfection efficiency was obtained, as assessed by visualizing the green fluorescence in cells transfected with pHYG-EGFP plasmid DNA (Clontech, CA) encoding enhanced green fluorescent protein. Later, Renilla luciferase in each cell extract served as an internal control for transfection.

Expression of SPA, SPA-mutant, and TLR4 proteins in HEK293 cells transfected with plasmid DNA constructs. The HEK293 cells transfected with pSPA or pSPA-mutant and pTLR4 constructs expressed SPA and TLR4 proteins (FIG. 33A). As expected, the molecular weight of the SPA-mutant protein expressed by HEK293 cells transfected with pSPA-mutant construct was smaller than the full-length SPA protein expressed by HEK293 cells transfected with pSPA construct (FIG. 33A). The secreted levels of SPA were also measured in cell-free supernatants by ELISA; 0.2 and 0.4 ng amounts of SPA per μg total cellular protein were measured in the supernatants of HEK293 cells transfected with pSPA and pSPA-mutant constructs, respectively (FIG. 33A).

TLR4 was expressed on the cell surface of the pSPA or pSPA-mutant and pTLR4 co-transfected HEK293 cells as observed by immunocytochemistry and flow cytometry (FIG. 33A); intracellular expression of TLR4 was also observed.

SPA4 peptide region is important for SPA-TLR4-interaction and inhibition of NF-κB activity. Consistent with the earlier Examples, it was observed that the SPA interacts with TLR4 in HEK293 cells. Confocal imaging revealed the co-localization of SPA and TLR4 in the cytoplasm as well as at the cell-surface (FIG. 33A). Importantly, the RLU values were reduced in cells co-transfected with pSPA-mutant and pTLR4 constructs as compared to the cells co-transfected with pSPA and pTLR4 constructs (FIG. 33B). The data demonstrate that the loss of SPA4 peptide region results into the reduction in SPA-TLR4 interaction.

The experiments were extended further to assess the effects of SPA- or SPA-mutant-TLR4 interaction on the LPS-induced NF-κB activity. The pGL4.32 NF-κB-reporter plasmid DNA was added to the two-hybrid assay system instead of pG5Luc plasmid DNA; the rest of the assay conditions were kept same. The cells were then incubated with LPS for 5 hours. As anticipated, the HEK293 cells transfected with pTLR4 plasmid construct showed a significant increase in NF-κB activity against LPS-stimuli (88 relative luminescence units; RLU, FIG. 33C). These results demonstrate that the LPS binds with TLR4 protein expressed by pTLR4-transfected cells and induces intracellular signaling. This increase in NF-κB activity was suppressed significantly in cells co-transfected with pSPA and pTLR4 constructs (88 versus 35 RLU; p<0.001). The cells co-transfected with pSPA-mutant and pTLR4 also showed suppressed NF-κB activity (88 versus 44 RLU; p<0.001). Although not significantly different, the NF-κB activity level was slightly increased in cells transfected with pSPA-mutant as compared to cells transfected with pSPA. These results demonstrate that reduction of interaction results in only partial inhibition of NF-κB activity.

Altogether the results of two-hybrid assay demonstrate that the SPA4 peptide region contributes to the SPA-TLR4 interaction. The two-hybrid assay results with SPA-mutant protein and TLR4 presented here confirm the findings of in silico protein-protein docking and peptide-screening analyses described earlier.

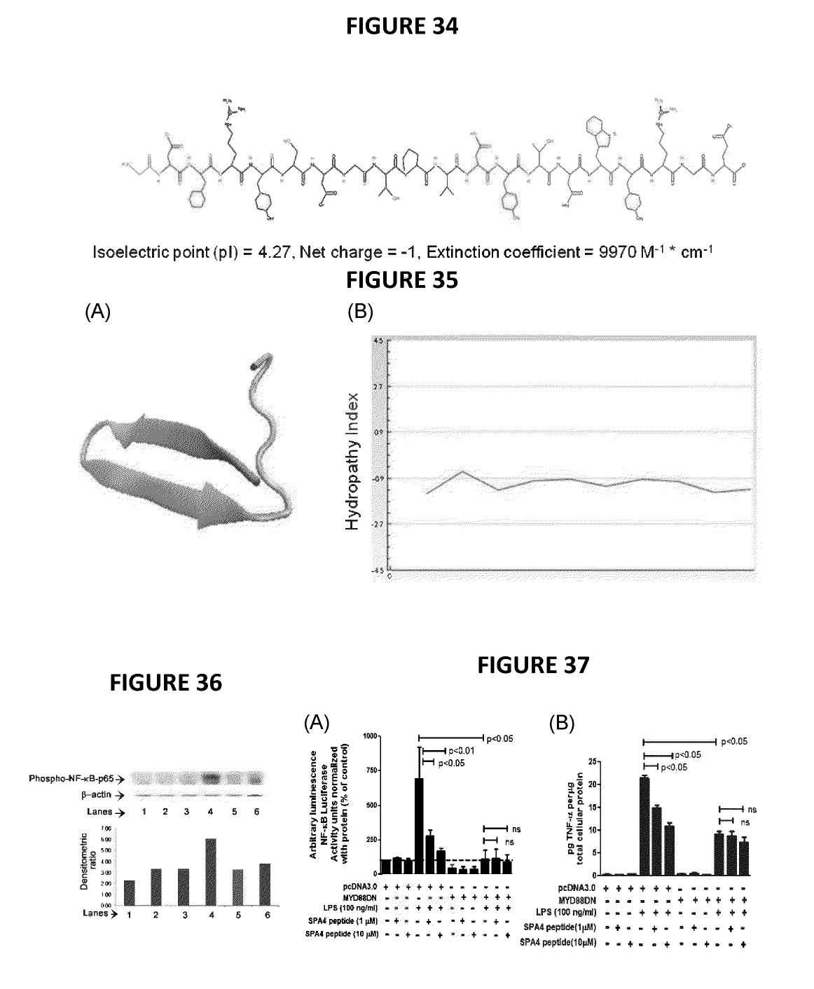

Physico-chemical characteristics of SPA4 peptide: Based on the results shown in earlier Examples and the results from two-hybrid assay presented here, the biological effects of the synthetic SPA4 peptide were then studied. The SPA4 peptide was synthesized by a commercial vendor (Genscript, NJ). Mass spectrograms and high performance liquid chromatograms confirmed the purity of each batch of synthetic SPA4 peptide (data not shown). The SPA4 peptide is predicted to have an isoelectric point of 4.27, net charge of −1, and extinction coefficient of 9970 M⁻¹*cm⁻¹ (FIG. 34). The predicted 3D structure of SPA4 peptide exhibits beta strands and coils (FIG. 35A). A plot of hydropathy index using the constants of Kyte and Doolittle indicates that the peptide is hydrophilic in nature (FIG. 35B).

As endotoxin is a well-characterized ligand for TLR4, the presence of endotoxin can significantly influence the results. Thus, all the solutions and reagents were prepared in endotoxin-free water, and all the assays were performed in an aseptic environment. The endotoxin was not detectable in purified SPA preparation, and was <0.04 pg per μg in SPA4 peptide suspensions.

SPA4 peptide inhibits LPS-induced NF-κB activity and TNF-α. Anti-inflammatory activity of SPA4 peptide was studied in an established JAWS II dendritic cell system. The NF-κB is a transcription factor that is induced by LPS-TLR4 via MYD88 and TIR-domain-containing adaptor-inducing interferon-β (TRIF)-dependent pathways. The activation of NF-κB, in turn, stimulates synthesis and secretion of pro-inflammatory cytokines. The effect of SPA4 peptide on LPS-induced NF-κB was determined by investigating the expression of phosphorylated-NF-κB-p65 and NF-κB-reporter activity in a dendritic cell line. The results show that the treatment with SPA4 peptide inhibits the LPS-stimulated phospho-NF-κB-p65 expression in JAWS II dendritic cells (FIG. 36). The results further revealed that the SPA4 peptide (1 and 10 μM) treatment significantly inhibited the LPS-induced MYD88-dependent NF-κB activity (FIG. 37A) and TNF-α release (FIG. 37B) in dendritic cells without any effect on the MYD88-independent NF-κB activity or the secreted levels of TNF-α. These results further support the inhibition of TLR4-induced inflammatory response by SPA4 peptide.

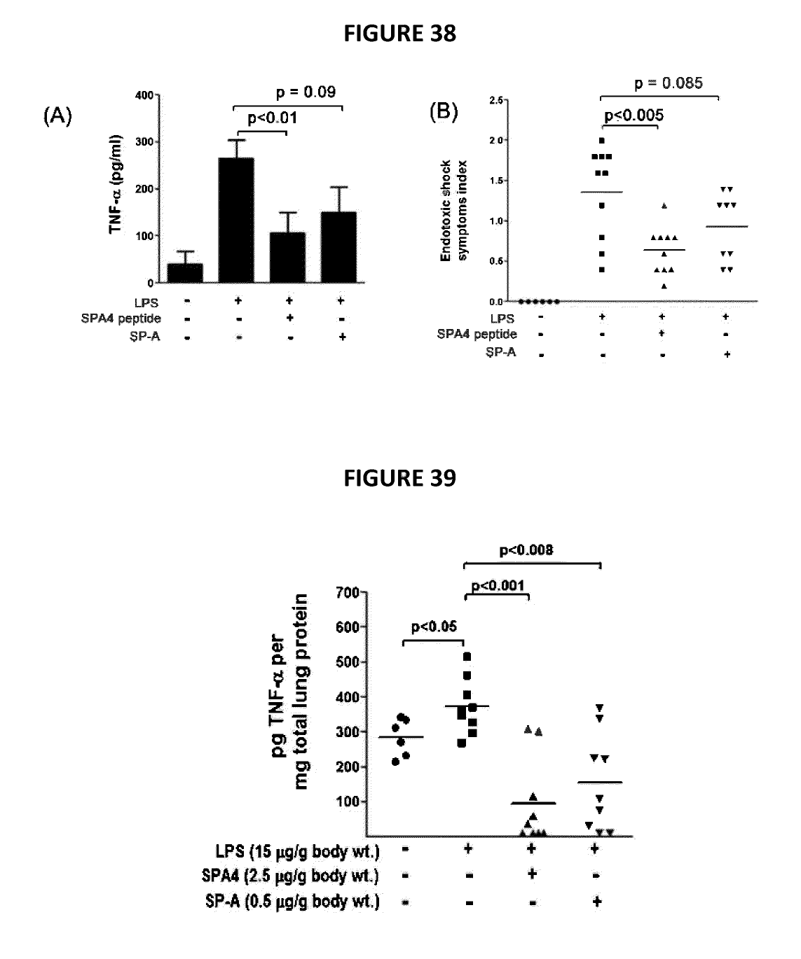

Biological effects of SPA4 peptide in a mouse model of LPS-induced lung inflammation. The activity of SPA4 peptide was then assessed by studying inflammatory parameters (nuclear localization of NF-κB-p65, TNF-α, influx of leukocytes) and endotoxic shock-like symptom indices in a mouse model of LPS-induced lung inflammation. Results were compared with those observed in SPA-treated mice. Inhibition of systemic TNF-α levels by SPA4 peptide translates to improvement in endotoxic shock-like symptoms. The circulating levels of LPS-induced TNF-α in mouse serum were significantly reduced after SPA4 peptide and SPA treatment (FIG. 38A). The inhibitory effect of SPA4 peptide on TNF-α was more pronounced than that of SPA (p<0.01 versus p=0.09).

An evaluation of the endotoxic shock-like symptom indices in animals revealed an alleviation of symptoms after treatment with SPA4 peptide and SPA. Endotoxic shock-like symptom index for each animal is demonstrated within FIG. 38B. The intraperitoneal challenge with LPS stimulated typical symptoms of endotoxic shock (mean symptom index 1.4) evident by ruffled fur (hair-raised and heterogenous), lack of reactivity and prostration (not reactive, difficulty in sitting, and rear legs tend to be extended), diarrhea (fluidy fecal matter stuck on fur), and eye exudate (exudates and eye closed). Treatment with SPA4 peptide and SPA led to a decrease in the LPS-stimulated endotoxic shock symptoms index (mean score 0.64 for SPA4 treated animals, p<0.005; mean score 0.93 for SPA treated animals, p=0.085).

SPA4 peptide suppresses the LPS-induced TNF-α, nuclear localization of NF-κB-p65, and leukocyte influx in lung. As expected, significantly increased levels of TNF-α were noted in lung homogenates of LPS-challenged mice (p<0.05, FIG. 39). The SPA4 peptide and SPA-treatment suppressed the LPS-induced TNF-α in lung homogenates (p<0.001 and p<0.008, FIG. 39). No significant differences were observed in MPO levels in lung tissue homogenates of LPS-challenged mice or in the LPS-challenged mice treated with SPA4 peptide or SPA (data not shown).

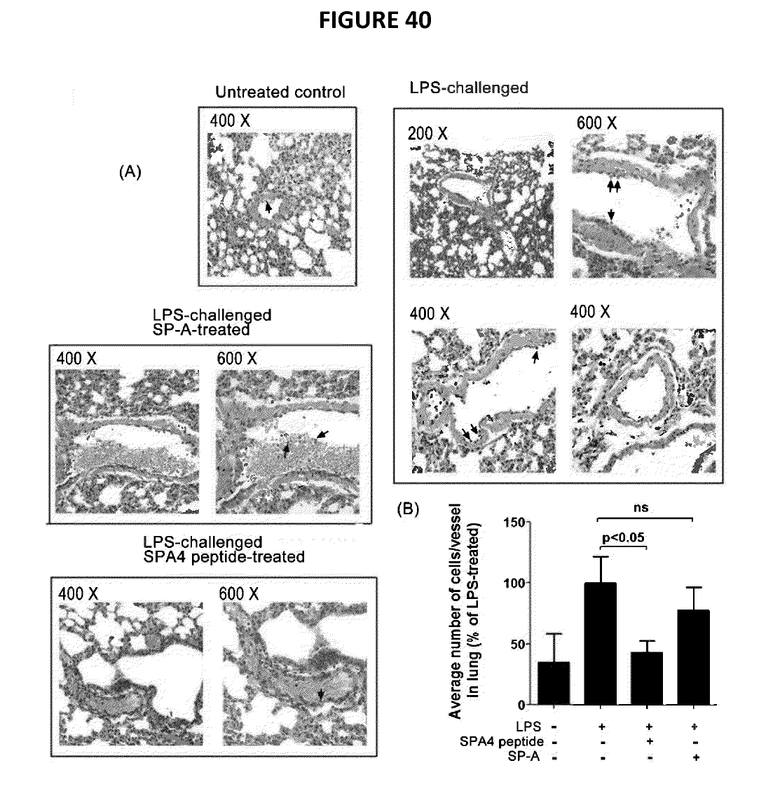

The H&E-stained lung sections were examined by a single-blinded, board-certified veterinary pathologist for the maximum lung damage present in each animal within the group. No or minimal damage was also reported. Firstly, LPS-induced histological changes in lung were noted primarily in the form of an accumulation of neutrophilic leukocytes within the lumen of the pulmonary vessel and/or pavemented along the endothelial lining (FIG. 40A). Secondly, the leukocytes present within the lumen of the pulmonary vessels were counted. In the LPS-challenged animals, more leukocytes were observed within the central part of the lumen and pavemented along the endothelial lining. The average number of leukocytes per vessel was set at 100% in LPS-challenged mice. Percent reduction in average number of cells was calculated for SPA4 peptide- and SPA-treated animal groups in comparison to those in LPS-challenged mice. On comparison, the lungs of SPA4 peptide-treated mice revealed a 50% reduction in the number of leukocytes per vessel. SPA treatment only resulted into 25% reduction of cell influx (FIG. 40B).

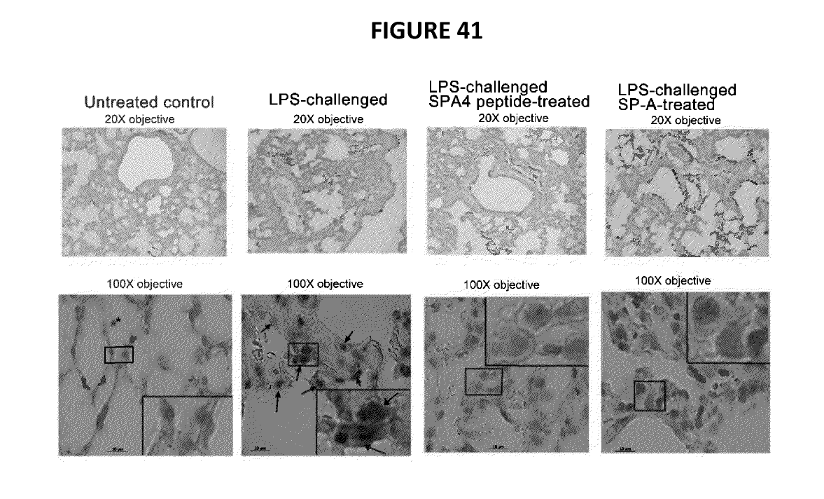

An intraperitoneal challenge with LPS stimulated the nuclear localization of NF-κB-p65 in mouse lung cells. It was observed that the LPS-induced nuclear staining of NF-κB-p65 was significantly reduced after treatment with SPA4 peptide and SPA (FIG. 41). However, the decrease in nuclear staining of NF-κB-p65 was more conspicuous in SPA4 peptide-treated mice than in the SPA-treated mice. The suppression of LPS-induced nuclear localization of NF-κB-p65 in lung cells was in agreement with the inhibitory activity of synthetic SPA4 peptide and purified lung SPA on other inflammatory parameters.

Discussion for Example 6:

Surfactant protein-A (SPA) plays an important role in host defense against a variety of pathogenic insults; SPA induces phagocytosis of bacterial and fungal pathogens, and suppresses the inflammatory response. As per the published results in animal models and patients, a decrease in the amounts of SPA in bronchoalveolar lavage fluids is associated with fulminant lung infection and inflammation; thus, the utilization of SPA as a therapeutic has been of a contemporary interest. In the past, it has not been possible to develop an SPA-based therapeutic or SPA-containing clinical surfactant because of the large size and hydrophilicity of SPA. In general, large-sized proteins tend to induce a non-specific immune response and are cleared rapidly; its hydrophilic nature also makes it difficult to mix SPA with hydrophobic lipids of clinical surfactants. An interesting study from Gardai et al., demonstrated that the N-terminal region of SPA can also induce pro-inflammatory effects against infectious challenge through its interaction with calreticulin. In view of these published results and formulation-related issues with full-length SPA, the small SPA fragments mimicking the beneficial host-defense characteristics of SPA can be of therapeutic use.

Example 1 demonstrated that the SPA interacts with TLR4, and SPA-TLR4 interaction suppresses the inflammatory response but maintains the phagocytic uptake of bacteria. These results corroborated with published reports in the literature. In Example 2, the peptide SPA4, derived from the C-terminal region of SPA, was identified and shown to suppress the release of TNF-α against LPS stimulus. This Example investigated the contribution of SPA4 peptide region in SPA-TLR4 interaction in HEK293 cells using a two-hybrid assay and evaluated the biological effects of synthetic SPA4 peptide in a dendritic cell system and in a mouse model of inflammation induced by the TLR4-ligand LPS. Presumably, the intraperitoneal LPS-challenge model in mice would closely mimic the pathological scenario as seen in patients with endotoxic shock-induced ARDS. It was anticipated that TLR4-signaling would be activated. Thus, the introduction of SPA4 peptide may help improve the host defense and alleviate the clinical symptoms.

To understand the activity of SPA4 peptide at cellular level, HEK293 cells and JAWS II dendritic cells were included. The HEK293 cells provided a cleaner system to test the SPA-TLR4 interaction without any interference from endogenous SPA and TLR4. The standard methods (co-localization, co-immunoprecipitation-immunoblotting) commonly employed for studying the protein-protein interaction have limited capacity of quantitating the affinity or avidity and identifying the regions involved in protein-protein interaction. Thus, the two-hybrid assay in HEK293 cells could provide a better alternative to identify interacting domains and regions. The results presented in this Example indicate that the two-hybrid assay provides quantitative measurement of protein-protein interaction and functional relevance, and allows investigation of particular domains and regions. The type II lung epithelial cells possess a highly-organized system for post-translational modification, packaging (e.g., lamellar body structures) and secretion of SPA. Although HEK293 cells may not have this machinery, a measurable quantity of secreted SPA was detected in the supernatants of pSPA and pSPA-mutant-transfected HEK293 cells (FIG. 33A). The work presented here in HEK293 cell system establishes that the SPA4 peptide region is important for interaction with TLR4 and inhibition of LPS-TLR4-induced NF-κB. Overall, these results demonstrate that the two-hybrid assay in HEK293 cells can provide a useful high-throughput tool for identifying other regions of SPA that bind to TLR4 and modulate immune responses.

As the HEK293 cells are not derived from peripheral mucosal sites and may not mimic the natural scenario, the biological activity of synthetic SPA4 peptide was assessed in vitro in a murine bone marrow-derived dendritic cell system and in vivo in a mouse model of LPS-induced lung inflammation. The results obtained in mouse dendritic cells reveal that the SPA4 peptide inhibits the LPS-stimulated phosphorylation of NF-κB-p65 unit, NF-κB activity, and TNF-α release in MYD88-dependent manner (FIGS. 36 and 37). It was also found that the SPA4 peptide does not bind to LPS. Overall, these results strengthen the broad hypothesis that the activity of SPA4 peptide against LPS stimuli (TLR4-ligand) is most likely through its interaction with TLR4 and not by sequestering its ligand: LPS. Detailed studies are required to delineate the mechanism of action of SPA4 peptide and other TLR4-interacting regions of SPA.

In Example 4, an established human colonic cancer epithelial cell line that constitutively expresses TLR4 was utilized; however, the biological effects of the SPA4 peptide in other TLR4-expressing cells and animal models remain to be explored. Thus, a mouse model of LPS-induced lung inflammation was included herein. The results reveal that the SPA4 peptide suppresses the LPS-induced inflammatory parameters (TNF-α, NF-κB activity and leukocyte influx) and alleviates LPS-induced symptoms. Interestingly, the anti-inflammatory effects of SPA4 peptide were equal to or more pronounced when compared to full-length SPA (FIGS. 38-41). Several possibilities exist, including the specific targeting of TLR4 and inhibition of inflammation by SPA4 peptide. Full-length SPA, however, can exert both pro-inflammatory and anti-inflammatory effects through a number of cell-receptors and mechanisms.(47-49) The mechanism of action of SPA4 peptide may differ from that of full-length SPA.

The results of this study are of clinical importance because an overwhelming inflammation leads to ARDS and multiple-organ failure, and an increased expression and activity of TLR4 has been linked with deleterious inflammatory response. Thus, the TLR4-interacting SPA4 peptide and other regions of SPA may have therapeutic potential in ARDS. Moreover, none of the clinical surfactants have SPA or SP-D.

### Example 7

### Pro-Phagocytoic and Anti-Inflammatory Activity of SPA4 Reduces Bacterial Burden and Inflammation in a Mouse Model of Pseudomonas aeruginosa Lung Infection

Antibiotic resistance and acquisition of new virulence traits by bacterial pathogens have contributed to an increase in the incidence of bacterial infections and associated morbidity and mortality. Lack of effective antibiotics makes it difficult to control infection and clinically manage patients. This has required the use of antibiotics which were formerly discarded because of their side effects. For example, colistin, an antibiotic introduced into clinical practice 50 years ago and abandoned due to nephrotoxicity, is now being used as a last-line treatment for antibiotic-resistant Gram-negative bacterial infections. Since infections are the leading cause of deaths worldwide, new therapeutic approaches with minimal or acceptable side effects are urgently needed to control Gram-negative bacterial infections.

Surfactant protein-A (SPA) is synthesized by epithelial cells at mucosal surfaces (lung, intestinal, and genitourinary) in our body. In the lung, secreted SPA helps maintain surface tension and normal lung function, and contributes to host defense. SPA can directly bind and kill a number of pathogens as well as enhance phagocytosis and clearance of pathogens by antigen presenting cells. Unfortunately, secreted levels of SPA are reduced in bronchoalveolar lavage fluids of patients as well as in animal models of lung infection. It is believed that replenishing SPA might help to restore homeostasis at the mucosal surface and aid in the elimination of pathogens. Despite better understanding of the host defense role of SPA, it has been difficult to utilize SPA for therapeutic purposes. Large-size and hydrophilicity of SPA have been major limitations for development of an SPA containing lipid-based surfactant product since large proteins are prone to degradation and rapid clearance in vivo. Also, the N-terminal region of SPA induces pro-inflammatory effects through its binding to calreticulin/CD91. It is conceivable that the SPA regions or domains can contribute to host defense in a distinct manner depending on the type of pathogenic stimuli, and interaction with pathogenic ligand and host cell receptors.

In certain embodiments, the presently disclosed inventive concepts are focused on harnessing the host defense properties of SPA by exploiting its interaction with Toll-like receptor 4 (TLR4). TLR4 is primarily expressed by immune cells, and its expression increases during infection. While TLR4 recognizes and phagocytoses pathogens, as well as coordinates the innate and adaptive immunity, uncontrolled activation of TLR4 leads to exaggerated inflammation. Example 1 demonstrated that the purified native lung SPA interacted with TLR4, promoted phagocytosis, and suppressed the inflammatory cytokine response against Gram-negative bacterial lipopolysaccharide (LPS) in dendritic cells. These findings led the inventor to consider whether short TLR4-interacting SPA-derived peptides can also exert pro-phagocytic and anti-inflammatory activity. Shorter SPA-derived peptides can overcome formulation issues and provide better therapeutic options. Earlier Examples revealed that the SPA4 peptide (GDFRYSDGTPVNYTNWYRGE; SEQ ID NO:3) bound to recombinant TLR4 protein in complex with myeloid differentiation protein 2 (MD2), and suppressed TLR4-induced inflammatory response against Gram-negative bacterial lipopolysaccharide (LPS; a potent ligand of TLR4) in cell systems and in a mouse model of LPS-induced endotoxic shock. This Example examines the effectiveness of the SPA4 peptide against live Gram-negative bacteria during infection.

In this Example, it was found that the SPA4 peptide does not directly interact or kill Escherichia coli and Pseudomonas aeruginosa but rather enhances uptake and intracellular trafficking of bacteria to acidic phagolysosomes, where lysis takes place through its interaction with TLR4. Also, the SPA4 peptide simultaneously decreases the TNF-α response to the pathogen. Furthermore, it was found that the SPA4 peptide treatment reduces bacterial burden and inflammation and alleviates clinical symptoms in a mouse model of P. aeruginosa lung infection.

Material and Methods for Example 7:

SPA4 peptide: SPA4 peptide (GDFRYSDGTPVNYTNWYRGE, SEQ ID NO:3)) was synthesized at Genscript, Piscataway, N.J. Fluorescein isothiocyanate was conjugated to SPA4 peptide (FITC-SPA4) at the N-terminal end (Genscript, Piscataway, N.J.). FITC was conjugated at the N-terminal end of the peptide through H-AHX(6)-OH(C₆H₁₃NO₂) spacer, and was not directly linked to any of the amino acids of the peptide, particularly not to the amino acids of the motif “NYTXXXRG” (SEQ ID NO:2), which were predicted to be in close proximity of TLR4. The fluorescence excitation and emission properties of the FITC-SPA4 were determined, and the steady state fluorescence spectrum was recorded with a Perkin Elmer fluorescence spectrometer (Perkin Elmer, Waltham, Mass.). The purity of each batch of peptide was confirmed by mass spectroscopy and high performance liquid chromatography (HPLC). SPA4 peptide was reconstituted in endotoxin-free water. Batch preparations of the peptide were checked for endotoxin contamination by Limulus amebocyte lysate assay (Charles River, Charleston, S.C.) as per the manufacturer's instructions.

Binding affinity of SPA4 peptide to recombinant TLR4-MD2 protein: In this Example, direct binding between FITC-SPA4 peptide and recombinant extracellular TLR4-MD2 protein (R & D Systems, Minneapolis, Minn.) was studied using a fluorescence polarization binding assay (Moerke, (2009) Curr Protoc Chem Biol. 1:1-15; and Liu et al. (2011) Chembiochem, 12:1827-1831.). The recombinant TLR4-MD2 protein encoded for human TLR4 (amino acids: Glu 24-Lys 631) and MD2 (amino acids: Glu 17-Asn 160). In principle, if the fluorescent FITC-SPA4 peptide will bind to TLR4-MD2 protein and get excited with the plane-polarized light, the resulting SPA4 peptide-TLR4-MD2 complex will tumble slowly in the solution, and the fluorescence emission will be polarized.

To assess the binding of SPA4 peptide to TLR4-MD2 protein, a fixed concentration of FITC-SPA4 peptide (2 μM) was incubated with 0-5.6 μM of recombinant TLR4-MD2 protein in the dark at room temperature within a total volume of 25 μl of 0.05 M sodium phosphate buffer (pH 7.0) (Qi et al. (2011) Enzyme research, 2011:513905). Changes in fluorescence polarization were measured as an indicator of FITC-SPA4 peptide binding to recombinant TLR4-MD2 protein at an excitation wavelength of 485 nm and an emission wavelength of 528 nm on a Synergy 2 multi-mode microplate reader (Biotek Instruments, Winooski, Vt.). Regression analysis was carried out using the Graphpad Prism program (Graphpad software, La Zolla, Calif.). Experimental data were curve-fitted using Graphpad Prism program, and the binding affinity (Kd) was determined.

Cell culture and maintenance: A murine derived JAWS II dendritic cell line (ATCC, Manassas, Va.) was used. These cells were maintained in alpha-modified minimum essential medium (α-MEM; Cellgro, Manassas, Va.) supplemented with 20% FBS, 100 U/ml penicillin, 100 μg/ml streptomycin, 50 μg/ml gentamicin (Life Technologies, Grand Island, N.Y.), and 5 ng/ml of recombinant murine granulocyte macrophage colony stimulating factor (Peprotech, Rocky Hill, N.J.).

Primary mouse alveolar macrophages: Alveolar macrophages were harvested from age- and sex-matched C57BL/6 mice (female, 5-6 week old) (Jackson Animal Laboratory, Bar Harbor, Me.). An angiocatheter was placed in the trachea and bronchoalveolar lavage fluid (BALF) was collected using ice-cold, endotoxin-, calcium- and magnesium-free Dulbecco's phosphate buffered saline (DPBS)(Awasthi et al. 2004. Respir Res 5:28). BALF was centrifuged at 400×g for 10 minutes at 4° C. Pelleted cells were suspended into RPMI 1640 medium containing 5% heat-inactivated FBS, 5 mM 4-(2-hydroxyethyl)-1-piperazineethanesulfonic acid (HEPES), 10 μg/ml gentamicin, 50 U/ml penicillin and 50 μg/ml streptomycin, and seeded at the density of 1×10⁵ cells per well in a 96-well tissue culture plate and incubated at 37° C. for 2 h. Nonadherent cells were removed, and adherent cells were washed multiple times prior to conducting the experiments.

Viability and morphologic characteristics of cells were monitored by the trypan blue dye exclusion method and Wright-Giemsa staining, respectively (Awasthi and Cox. 2003. Biotechniques 35:600-602, 604).

Characteristics of bacterial strains: Escherichia coli 19138 serotype O6:K2:H1 (ATCC, Manassas, Va.), Pseudomonas aeruginosa PAO1 (Raymond et al. 2002. J Bacteriol. 184:3614-3622) and green fluorescent protein (GFP)-expressing P. aeruginosa 8830 (GFP-P. aeruginosa; obtained from Dr. William McShan, Department of Pharmaceutical Sciences, College of Pharmacy, OUHSC, OK) strains were used (Olvera et al. 1999. FEMS microbiology letters 179:85-90). These bacterial strains have been previously used to induce infection in mice. The bacterial cultures were maintained in Luria-Bertani (LB), tryptic soy or nutrient broth or agar media.

Competent E. coli 19138 was transformed with 1 μg of plasmid DNA encoding GFP (pGFPuv; Clontech Lab, Mountain View, Calif.; obtained from Dr. Nathan Shankar, Department of Pharmaceutical Sciences, College of Pharmacy, OUHSC, OK) as per the published method (Crameri et al. 1996. Nat Biotechnol 14:315-319). Biochemical characteristics of P. aeruginosa and E. coli strains were determined by using oxidase and catalase reagents (BD Biosciences, San Jose, Calif.), per the manufacturer's instructions. Gram staining was performed to ensure the purity of the bacterial cultures.

Bacterial growth curve: Bacteria were grown in culture medium overnight at 37° C. with shaking. The overnight bacterial cultures were sub-cultured in pre-warmed fresh medium and incubated at 37° C. An aliquot of culture was removed at different time intervals and its optical density was read at 600 nm (OD₆₀₀). At the same time, an aliquot of bacterial culture was serially diluted in sterile Dulbecco's Phosphate Buffered Saline (DPBS; Life Technologies, Grand Island, N.Y.) and plated onto agar plates (Jett et al. 1997. Biotechniques 23:648-650). Bacterial colonies were counted, and a linear regression equation was determined by plotting CFU/ml versus OD₆₀₀. For subsequent experiments, the bacterial cultures were collected from the mid-logarithmic phase of growth curve after 2-6 hours of sub-culturing.

Phagocytosis assay: Dendritic cells were suspended in endotoxin-free phagocytosis assay buffer containing 1 mM CaCl₂, 1 mM MgCl₂ and 1 mM HEPES. The phagocytosis assay buffer was optimized to maintain cell viability (90-95%), size and volume as evidenced by the trypan blue staining and flow cytometric forward and side scatter pattern of the cells, respectively. One million dendritic cells were incubated with GFP-E. col±19138 or GFP-P. aeruginosa 8830 at different multiplicities of infection (MOI; cell to bacteria ratio) with or without 1% normal mouse serum for optimization. Different amounts of SPA4 peptide were added to the cell and bacterial mix. The reaction mix was incubated at 37° C. for 45 minutes while shaking, and swirled after every 15 minutes. Cells were finally washed three times with DPBS to remove free bacteria, and run on an Accuri flow cytometer (BD Biosciences, San Jose, Calif.). Cytochalasin D (Sigma, St. Louis, Mo.) served as negative control. The cells and bacteria alone provided additional controls for flow cytometric analysis (BD Accuri C6 program). The flow cytometric pattern of cells and bacteria was used for setting the gates. Any shift in the FL1 (green) fluorescence of the gated cells was considered for bacterial phagocytosis.

Transient transfection of dendritic cells with wild-type TLR4 plasmid DNA construct: Dendritic cells (1×10⁶ cells per well) were transfected with 2 μg of each plasmid construct: pDisplay control vector or the plasmid construct encoding wild-type mouse TLR4 on the same vector backbone, using the TranslT-TKO transfection reagent (Mirus, Madison, Wis.). Both plasmid constructs were obtained from Dr. Lynn Hajjar, Department of Comparative Medicine, University of Washington, Seattle, Wash. (Jou et al. 2006. Am J Pathol 168:1619-1630). The ratio of transfection reagent to plasmid DNA (2 μl per 1 μg of DNA) was kept constant. After 4 hours of incubation, cells were supplemented with an additional 250 μl of α-MEM medium containing 20% plain FBS and incubated for an additional 14-16 h. Transiently-transfected cells were washed and scraped gently, and re-seeded at the density of 10⁵ cells per well in Opti-MEM reduced-serum medium (Life Technologies, Grand Island, N.Y.).

pHrodo-labeled E. coli and P. aeruginosa: After the bacteria are taken up by the antigen-presenting cells, the bacteria are internalized in the endosome structures. The endosomes are then fused with lysosomes and form phagolysosomes, where bacterial lysis takes place. The pHrodo dye fluoresces only at an acidic pH. This property has been used to investigate localization of bacteria inside the acidic phagolysosomes. As shown in the previous Examples, pHrodo-labeled bacteria did not fluoresce outside the cells, and the labeled bacteria fluoresced red only when inside the cells. Therefore, this assay specifically determined the intracellular localization of bacteria in acidic compartments of the cells.

In this Example, commercially-available pHrodo-conjugated E. coli K12 (Life Technologies, Grand Island, N.Y.) was employed. The heat-killed P. aeruginosa PAO1 were conjugated to pHrodo dye as per the manufacturer's instructions. P. aeruginosa PAO1 was heat-killed at 65° C. for 10 minutes and washed twice with DPBS. Bacterial cells (1.2-1.5×10⁹ CFU) were suspended in 300 μl of freshly-prepared 100 mM sodium bicarbonate solution (pH 8.5). The bacterial suspension was then incubated with 0.93 mM pHrodo succinimidyl ester (Life Technologies, Grand Island, N.Y.) solution for 45 minutes at room temperature in the dark. The bacteria were washed with Hank's Balanced Salt Solution (HBSS). The pHrodo-labeled bacterial pellet was washed in 1 ml of 100% methanol and HBSS before suspending in HBSS containing 20 mM HEPES. The pHrodo-labeled bacteria were sonicated at room temperature to remove aggregates. Freshly reconstituted pHrodo-labeled E. coli or freshly prepared pHrodo-labeled P. aeruginosa were used for studying their intracellular localization inside the phagolysosomes.

Localization of bacterial particles in the acidic compartments of the cells: Nontransfected or genetic-transfected JAWS-II dendritic cells and alveolar macrophages were seeded in Opti-MEM reduced-serum medium (Life Technologies, Grand Island, N.Y.) at a density of 1×10⁵ cells per well. Sonicated pHrodo-conjugated E. coli or P. aeruginosa (one dendritic cell to 300-680 bacteria) were added to the cells. After 1.5 hours of incubation, the cells were treated with 75 μM SPA4 peptide, the maximum concentration used in the flow-cytometric phagocytosis assay. Fluorescence readings were then taken at 3.5 hours of incubation at 530 nm excitation and 590 nm emission wavelengths, using the Synergy 2 multi-mode microplate reader (Biotek, Winooski, Vt.).

The fluorescence readings of the pHrodo-conjugated bacteria alone were subtracted from the control (basal without any effector molecules) and experimental phagocytosis reaction wells. Finally, the percent localization of pHrodo-conjugated bacteria into acidic phagolysosomes was calculated using the following formula:

Percent localization=(localization in control or experimental wells/localization in control wells)×100

The cell-free supernatants were collected after taking the fluorometric readings, and stored at −80° C. for further analysis. The cellular protein content was measured by the Bicinchonic acid assay (BCA) assay as per the manufacturer's protocol (Pierce Biotechnology, Rockford, Ill.) and was used to normalize the fluorometric measurements.

Confocal microscopy: After the assessment of phagocytosis or localization of bacteria inside the acidic compartments by flow cytometry or fluorometry was completed, representative samples of cells were transferred to glass chamber slides (Nunc, Rochester, N.Y.) and fixed in 3.5% paraformaldehyde solution for 20 minutes on ice. The fixed cells were washed three times with ice-cold DPBS and stained with the Hoechst 33342 dye (1 μg/ml) for nuclear staining. The slides were mounted with Vectashield (Vector Laboratories Inc, Burlingame, Calif.) and examined by confocal microscopy.

Lysosomal-associated membrane protein 1 (LAMP1) is expressed on lysosomal structures in cells, including phagolysosomes. Thus, representative samples of cells were fixed in 3.5% paraformaldehyde solution for 20 minutes on ice in 8 well chamber slides (Nunc, Rochester, N.Y.) to confirm localization of bacteria within LAMP1-expressing phagolysosomes. Fixed cells were permeablized with 0.05% saponin solution, and non-specific sites were blocked with 1% bovine serum albumin. Alexa Fluor 488-conjugated anti-mouse LAMP1 antibody (25 μg/ml; Biolegend, San Diego, Calif.) was added to the cells. After 2 hours of incubation in a humidified chamber in the dark, cells were washed and Hoechst 33342 dye (1 mg/ml) was added for nuclear staining. All the images were acquired using a 63× oil immersion objective in a Zeiss confocal microscope and processed using the Zeiss ZEN 2011 program.

Direct binding of SPA4 peptide to bacteria: It was also examined whether the SPA4 peptide directly binds to live bacteria and affects bacterial growth outside of the cells. Bacterial cells harvested from 4 hours mid-logarithmic growth phase were washed in endotoxin-free phagocytosis assay buffer, and incubated with 10, 50, 75 and 100 μM of FITC-SPA4 peptide. The reaction mix was incubated at 37° C. on a shaking water bath (85 rpm) for 45 min, and swirled after every 15 min. Bacterial cells were washed three times with DPBS to remove free FITC-SPA4 peptide, and then run on an Accuri flow cytometer (BD Biosciences, San Jose, Calif.). Polymyxin B is a cyclic cationic peptide antibiotic that binds to anionic lipids, specifically the Gram-negative bacterial LPS. The Oregon green 514-conjugated polymyxin B (Life Technologies, Grand Island, N.Y.) was included as a positive control for binding to the bacteria.

The binding of FITC-SPA4 peptide or Oregon green 514-conjugated polymyxin B to bacterial cells was then studied by confocal microscopy. Bacterial suspensions treated with FITC-SPA4 peptide or Oregon green 514-labeled polymyxin B were air dried on a glass slide and mounted with Vectashield (Vector Laboratories, Burlingame, Calif.). Since the fluorescence spectrum was slightly different for FITC-SPA4 peptide (excitation 488 nm, emission 515 nm) than that of Oregon green 514-labeled polymyxin B (excitation 514 nm, emission 532 nm; Life Technologies, Grand Island, N.Y.), the emission capture was set at 493-523 nm for FITC and 519-646 nm for Oregon green dye in the confocal microscope. All of the images were acquired with 63× oil immersion objective in a Zeiss confocal microscope and processed using the Zeiss ZEN 2011 program. Bacteria alone without any treatment with SPA4 peptide or polymyxin B served as a negative control.

The direct antibacterial activity of SPA4 peptide was assessed as previously described (Kim et al. 2002. J Immunol 168:2356-2364). An aliquot of mid-logarithmic culture of E. coli and P. aeruginosa was pelleted at 5,000×g at 4° C. and washed with sterile DPBS. Diluted bacterial suspension was added to the wells of a Honeycomb 2 plate (Oy Growth Curves Ab Ltd., Helsinki, Finland) which contained 1, 10, or 100 μg/ml SPA4 peptide or an equivalent amount of vehicle. Positive controls contained antibiotics (ampicillin for E. coli or kanamycin for P. aeruginosa). Absorbance readings (OD₆₀₀) were taken at 37° C. every 15 minutes for 17 hours using the BioScreen C (Oy Growth Curves Ab Ltd., Helsinki, Finland). After 17 hours of incubation, an aliquot of the culture was serially diluted in DPBS, plated on agar plates, and incubated overnight at 37° C. Bacterial colonies were counted to obtain CFU/ml.

Evaluation of SPA4 peptide in a mouse model of P. aeruginosa lung infection: Animal experiments were approved by the Institutional Animal Care and Use and Biosafety Committees at the University of Oklahoma Health Sciences Center (OUHSC), Oklahoma City, Okla. C57BL/6 mice (female, 5-6 weeks old; Jackson Animal Laboratory, Bar Harbor, Me.) were included and housed for 1 week for acclimatization prior to conducting any experiments.

Fresh log phase cultures of P. aeruginosa PAO1 were harvested by centrifugation, washed, and suspended in sterile saline. Bacterial inoculums were confirmed by plating serial 10-fold dilutions of bacterial suspension on agar plates. Mice were anaesthetized, positioned on the intubation platforms, and challenged with bacteria intratracheally using a gel-loading pipette tip and laryngoscope (Penn-Century, Wyndmoor, Pa.). After one hour of bacterial challenge, SPA4 peptide (50 μg) was administered intratracheally. Symptoms and reactivity to tail-holding stimulus were scored and recorded after 5 hours of bacterial challenge. Blood and whole lung or lung tissue pieces were collected aseptically at the time of necropsy. The lungs were weighed and homogenized. Serially-diluted lung homogenates were plated on agar plates and incubated at 37° C. Bacterial colony forming units (CFU) were noted and normalized with wet lung weight. Protease inhibitors (1 mM EDTA, 1.1 μM leupeptin, 1 μM pepstatin, 0.2 mM phenylmethyl sulphonyl fluoride) and detergents (0.1% SDS, 1% Igepal CA630) were added to homogenization buffer to prepare lung tissue homogenates. The lung tissue homogenates were stored frozen at −80° C. for TNF-α cytokine analysis as described below. In separate sets of experiments, the lung tissue pieces were fixed in 10% formalin, transferred into 70% ethanol, sectioned and stained with hematoxylin and eosin. Tissue sections stained with hematoxylin and eosin were examined for the extent of tissue damage and inflammatory cells by a board-certified veterinary pathologist at the OUHSC.

Cytokine response: TNF-α levels were measured in cell-free supernatants or lung tissue homogenates as previously described (Vilekar et al. 2012. Int Immunol 24:455-464). Levels of secreted TNF-α were normalized to total cellular or tissue protein content.

Statistics: Statistical significance was analyzed using the Student's t-test or ANOVA (Prism software, La Zolla, Calif.). Statistical significance was defined as a p value of ≦0.05 or otherwise indicated.

Results of Example 7:

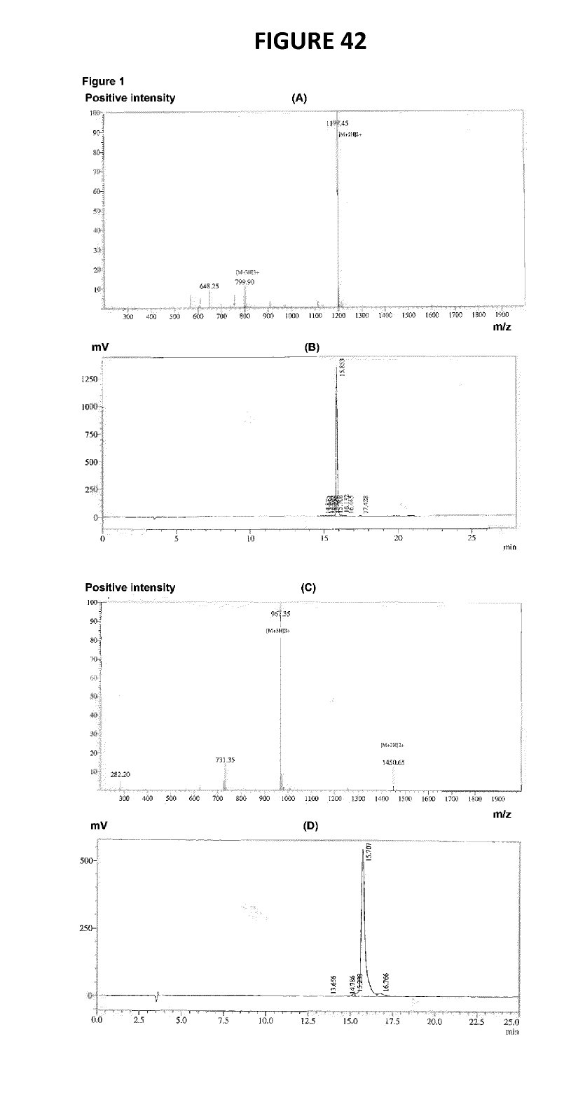

The bacterial strains included in the study were characterized for catalase and oxidase activity, as well as Gram-negative staining. As expected, E. coli colonies were catalase positive and oxidase negative, while P. aeruginosa colonies were positive for both catalase and oxidase enzymes. All of the bacterial strains maintained Gram-negative staining, and colony and growth characteristics throughout the study. The HPLC and mass spectrograms confirmed the purity of SPA4 peptide and FITC-SPA4 peptide (FIG. 42).

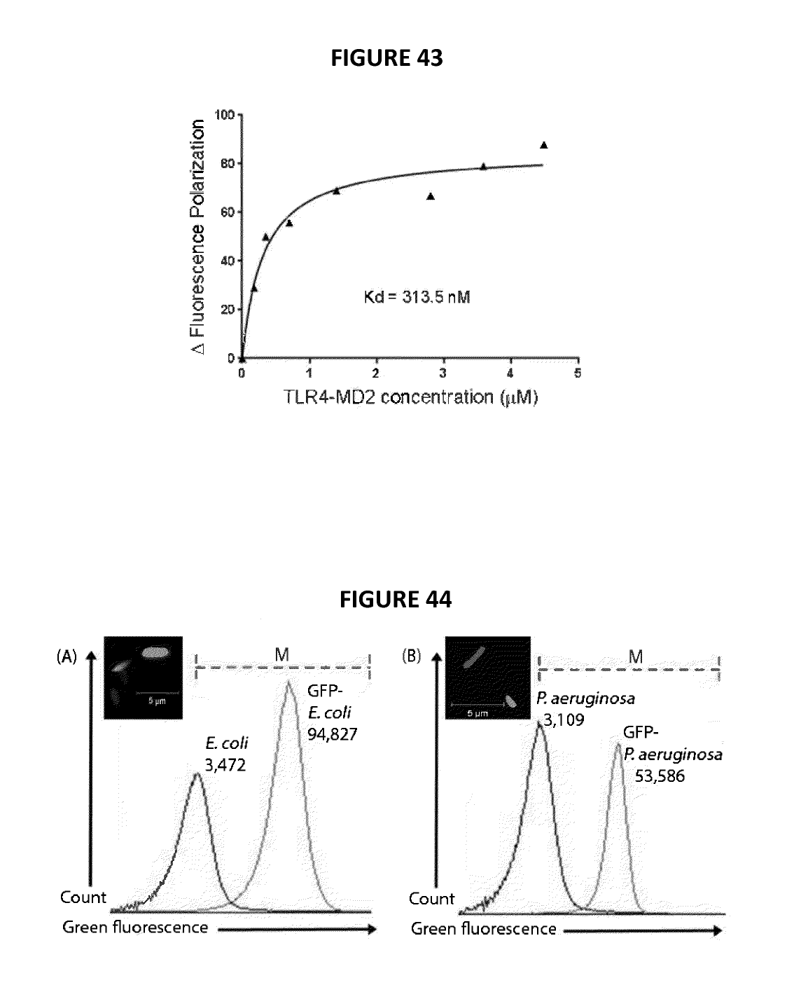

SPA4 peptide binds to extracellular TLR4-MD2 protein. In the previous Examples, in silico analysis, immunoassay, and a mammalian two-hybrid approach were utilized to demonstrate that the SPA4 peptide region of SPA is the key TLR4-binding site. In this Example, an in vitro biophysical binding assay was developed to test direct binding of FITC-SPA4 peptide to recombinant TLR4-MD2 protein based on the changes in fluorescence polarization as described above. The FITC-SPA4 peptide in bound form with TLR4-MD2 protein polarized light, and an increase in polarization values was noted in the direct binding assay. FITC was conjugated at the N-terminal end of the SPA4 peptide through a linker; thus it was speculated that FITC conjugation would not have affected the binding of SPA4 peptide. The polarization values of FITC-SPA4 peptide incubated with recombinant TLR4-MD2 protein were subtracted from the background value (without any FITC-SPA4 peptide) and curve-fitted using the Graph pad Prism program. The coefficient of determination of binding was noted to be >0.95, indicating a good fit. Lower values of the Kd indicate strong binding affinity. Average binding affinity (Kd) of FITC-SPA4 peptide was 0.255 μM±0.06 (SEM) without blank subtraction, 0.407 μM±0.09 with blank subtraction at a 75 arbitrary unit of sensitivity. Representative binding curve is shown in FIG. 43. These results demonstrate that the SPA4 peptide has relatively strong binding affinity to recombinant TLR4-MD2 protein.

SPA4 peptide enhances bacterial phagocytosis. TLR4 is important for pathogen-recognition, phagocytosis, and inflammation. Thus, it was determined whether SPA4 peptide binding to TLR4 alters the bacterial phagocytosis by dendritic cells. Dendritic cells were incubated with GFP-E. coli or GFP-P. aeruginosa in the presence or absence of SPA4 peptide, and the cells were washed and phagocytosis measured by detecting the shift in cell-associated green fluorescence by flow cytometry. Results were confirmed by visualization of intracellular phagocytosed bacteria using confocal microscopy.

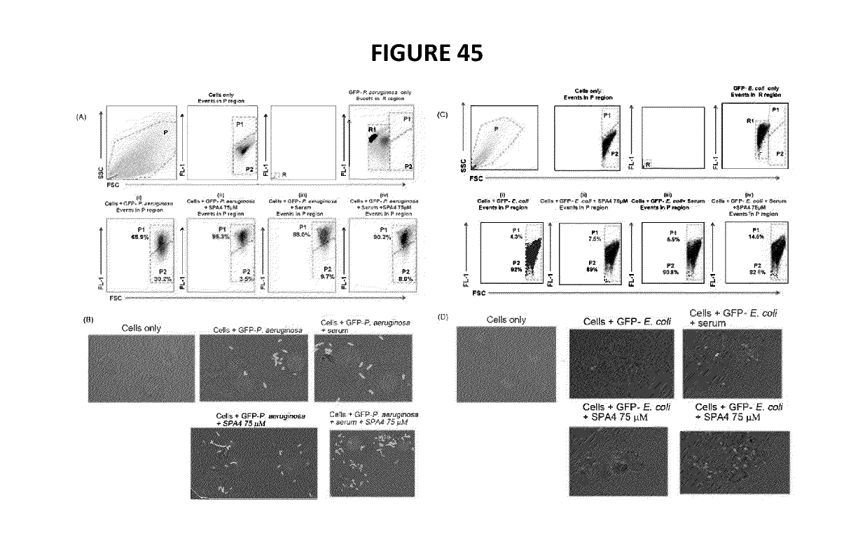

First, the stability of GFP-expression by GFP-E. coli and GFP-P. aeruginosa was confirmed under the phagocytosis assay conditions. A shift in the flow cytometric histogram of live bacteria in the FL1 channel and green fluorescent colonies confirmed GFP expression by GFP-E. coli and GFP-P. aeruginosa (FIGS. 44A, 44B). At the same time, the flow cytometric histogram of the JAWS II dendritic cells used in the assay was noted in the FL-1 channel for each experiment. The histogram plots of GFP-expressing bacteria and dendritic cells were well-separated in the FL-1 channel, and were stable over time. Therefore, any shift in the histogram plot of dendritic cells when incubated with GFP-expressing bacteria indicated uptake of GFP-expressing bacteria.

Next, the effect of SPA4 peptide on phagocytosis of bacteria was examined using the JAWS II murine dendritic cell line. Phagocytosis was determined after incubation of cells with GFP-bacteria for 45 minutes. Uptake of GFP-bacteria was indicated by a shift in the flow cytometric histogram of dendritic cells. The assay was performed in phagocytosis assay buffer optimized in order to maintain cell size and volume as assessed by their forward and side scatter flow cytometry properties. In the initial experiments, the MOIs (cell to bacteria ratios) used were 1:25, 1:50, and 1:100, and SPA4 peptide concentrations used were 10, 50 and 75 μM. The MOI of 1:100 and SPA4 peptide concentration of 75 μM were used for comprehensive experiments in the absence and presence of 1% normal mouse serum. Experiments under these conditions consistently demonstrated that the SPA4 peptide induced bacterial phagocytosis by dendritic cells (FIGS. 45A-D).

To confirm the results determined by flow cytometry, representative samples were analyzed by confocal microscopy (FIGS. 45B, 45D). A Z-stack of cells was captured using confocal microscopy. The percentage of cells with internalized GFP-bacteria (phagocytosed) was noted at the specific plane of the Z-stack when the cell nucleus was visible. Analysis using confocal microscopy showed an increase in the percentage of cells with phagocytosed bacteria after SPA4 peptide treatment; these results were consistent with the flow cytometry analysis. Very few bacteria were observed towards the outer edge of the cells; these bacteria could be tightly attached to the outside of the cell. Therefore, it was determined whether these measurements reflected phagocytosis activity, or whether they could be affected by tightly bound external bacteria. Treatment with cytochalasin D reduced the percentage of cells with green fluorescence to 40% as compared to 100% basal phagocytosis of GFP-bacteria without any effector molecule as assessed by flow-cytometry. This demonstrates that at least 60% of the cell-associated fluorescence measured in the assays was due to phagocytosed, and not just cell-bound bacteria.

SPA4 peptide enhances localization of pHrodo-conjugated E. coli and P. aeruginosa into phagolysosomes and suppresses the release of TNF-α. The effects of SPA4 peptide on bacterial uptake were further investigating by observing the localization of bacteria in the LAMP1-expressing acidic phagolysosomes within the dendritic cells. Heat-killed E. coli and P. aeruginosa bacteria labeled with pHrodo dye, which fluoresces only at an acidic pH and reveals localization within acidic compartments of the cells, including LAMP1-expressing phagolysosomes, were utilized. Expression of LAMP1 was detected using an Alexa Fluor 488-conjugated anti-mouse LAMP1 antibody (see Materials and Methods). The fluorescence associated with the localization of bacteria inside the acidic phagolysosomes was measured after 3.5 hours of infection.

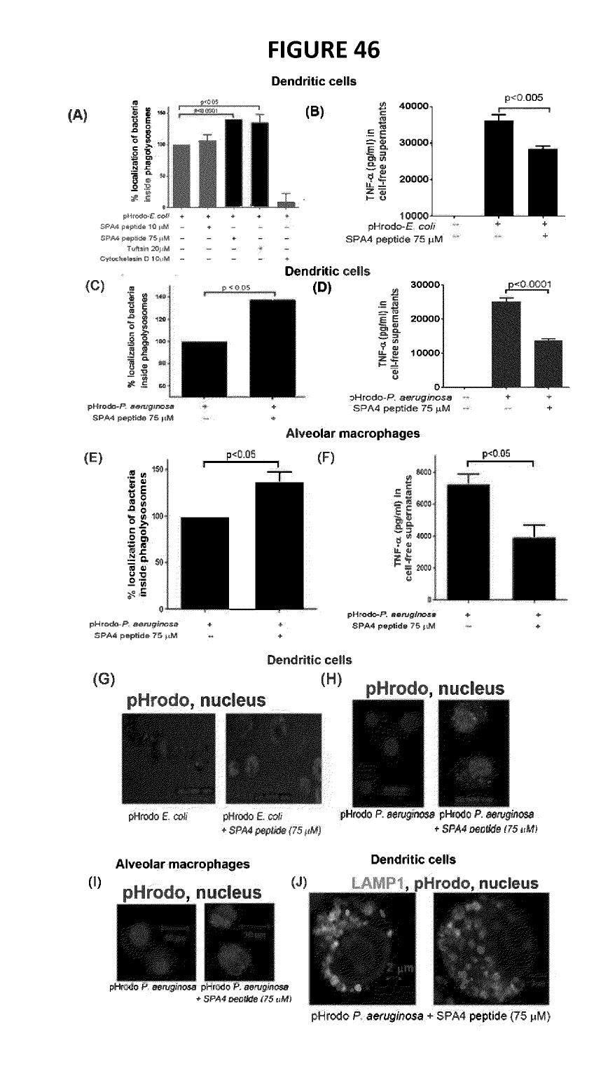

SPA4 peptide treatment enhanced localization of E. coli and P. aeruginosa in the acidic compartments of dendritic cells and macrophages by 10-40% as compared to basal phagocytosis (without any effector molecule). Tuftsin was included as positive control which enhanced localization by 35% as compared to untreated cells (FIG. 46). Cytochalasin D, an inhibitor of actin polymerization, significantly inhibited the phagocytosis and trafficking of bacteria to the acidic compartments, as expected (FIG. 46). Localization of bacteria (red fluorescence) inside the LAMP1-expressing phagolysosomes (green fluorescence) was confirmed by confocal microscopy (FIG. 46).

Secreted levels of TNF-α were also measured in the supernatants of dendritic cells and alveolar macrophages challenged with pHrodo-labeled E. coli and P. aeruginosa. It was found that the SPA4 peptide suppressed the TNF-α levels after challenge with E. coli or P. aeruginosa (FIG. 46).

Altogether, these results show the promising effects of SPA4 peptide-enhanced phagocytosis and trafficking of bacteria to phagolysosomes, while decreasing the levels of inflammatory cytokine.

Pro-phagocytic and anti-inflammatory activity of SPA4 peptide is through its interaction with TLR4. In order to determine whether these effects of SPA4 peptide are through TLR4, SPA4 peptide activity was evaluated in dendritic cells overexpressing TLR4. For these experiments, the dendritic cells were transfected with a plasmid DNA encoding wild-type TLR4. First, it was assessed whether overexpression of TLR4 increased the dendritic cell response to a TLR4-ligand, E. coli-derived LPS, using methods described in the previous Examples. Briefly, dendritic cells were co-transfected with plasmid constructs encoding wild-type mouse TLR4 and NF-κB luciferase reporter plasmid DNA. Cells were then challenged with LPS (100 ng/ml) for 4 h, and luciferase reporter activity was measured as an assessment of NF-κB activation. As expected, overexpression of TLR4 increased NF-κB activation by LPS as compared to control vector-transfected cells (results not shown). These results demonstrate that overexpression of TLR4 in cells increases its downstream effector function.

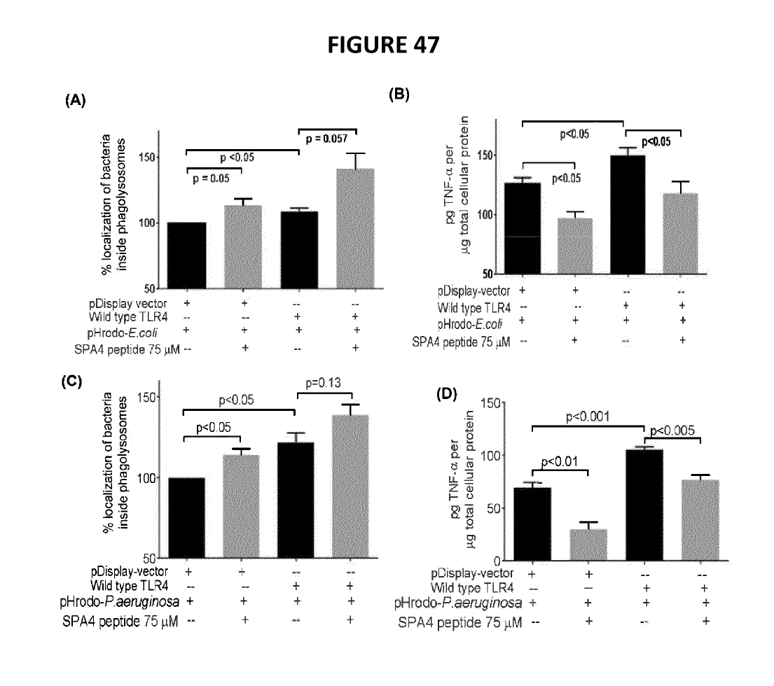

It was also found that increased expression of TLR4 increased localization of pHrodo-labeled bacteria in acidic phagolysosomes and also enhanced the secreted levels of TNF-α. Treatment of TLR4-transfected cells with SPA4 peptide maintained the TLR4-induced bacterial uptake and intracellular trafficking for lysis (FIGS. 47A, 47C), and yet significantly decreased the secreted levels of TNF-α (FIGS. 47B, 47D). These results demonstrate that the pro-phagocytic and anti-inflammatory activities of SPA4 peptide are mainly through its interaction with TLR4.

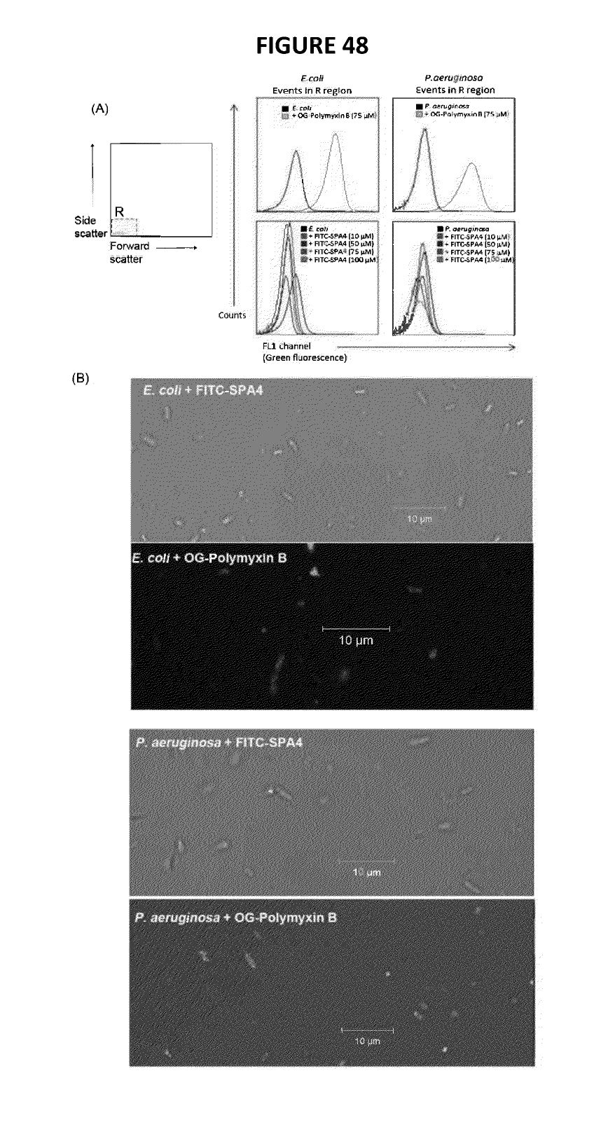

SPA4 peptide neither directly binds to bacteria nor affects bacterial growth. Full-length native SPA binds to a number of pulmonary pathogens, including Gram-negative bacteria, and directly kills the pathogens by increasing membrane permeability.(32) Thus, it was assessed whether SPA4 peptide mimics the SPA function of directly binding to the bacteria and suppressing bacterial growth. Direct binding of the FITC-SPA4 peptide to live bacterial cells was assessed by flow cytometry and confocal microscopy. Polymyxin B binds strongly to LPS in bacterial cell walls. Incubation of bacterial cells with Oregon green 514-conjugated polymyxin B caused a significant shift in the fluorescence peak in flow cytometric histograms indicating binding (FIG. 48A). However, no shift was observed when FITC-SPA4 peptide was incubated with live bacterial cells. Bright green fluorescence of Oregon green 514-conjugated polymyxin B bound to bacteria was clearly visible in confocal images (FIG. 48B).

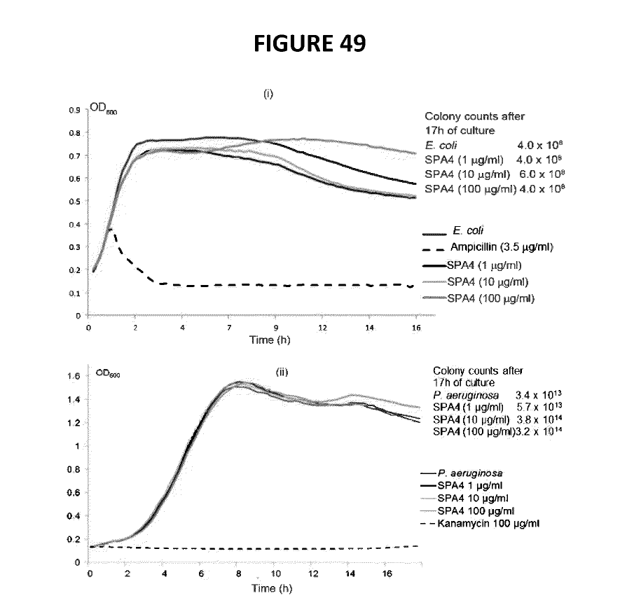

The effect of SPA4 peptide on the growth of E. coli 19138 and P. aeruginosa PAO1 was also measured. Mid-log phase bacteria were cultured in liquid culture medium in the presence of SPA4 peptide or vehicle. Addition of the SPA4 peptide did not affect subsequent bacterial growth, as assayed by OD₆₀₀ or colony counts (FIG. 49). These findings demonstrate that the SPA4 peptide neither binds to the bacteria nor directly kills or affects bacterial growth.

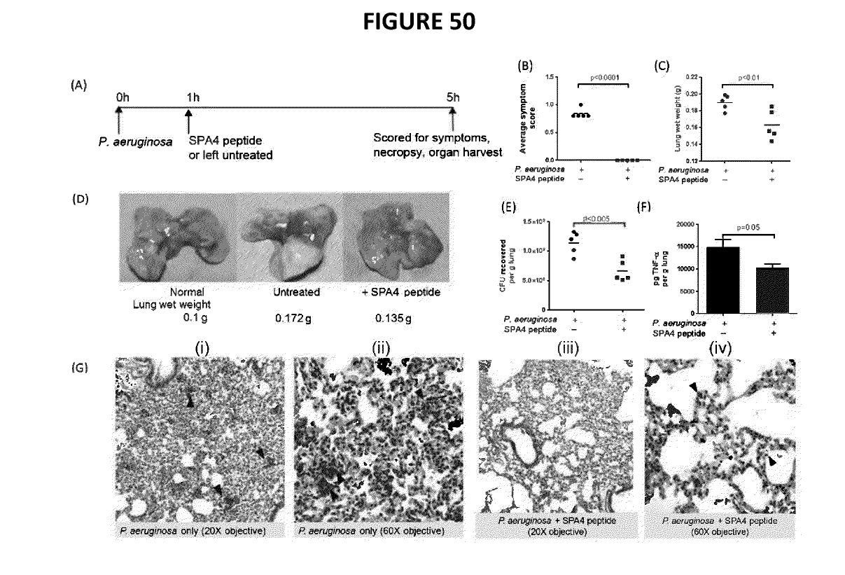

SPA4 peptide treatment reduces bacterial burden, TNF-α, and tissue damage in lungs of P. aeruginosa challenged mice. Next, it was tested whether pro-phagocytic and anti-inflammatory activity of SPA4 peptide tested in vitro translates to improvement of host defense in vivo. C57BL/6 mice were challenged with 1×10⁷−1×10⁸ viable CFU of P. aeruginosa. Previous Examples have demonstrated that the SPA4 peptide is effective when given post LPS challenge. Thus, this therapeutic model was used for in vivo studies, and the mice were treated with SPA4 peptide 1 hour after the infectious challenge. Clinically, it was observed that mice treated with SPA4 peptide were more alert and reactive to tail-holding stimuli and had less symptoms as compared to untreated infected mice (FIG. 50B, p<0.0001). SPA4 peptide treatment also decreased lung injury in infected mice, as evidenced by reduced lung wet weight compared to untreated controls (FIG. 50C, D; p<0.01). A single dose of SPA4 peptide also reduced bacterial burden by 1-1.5 logs and decreased the TNF-α response by about half in lungs of infected mice after 5 hours of infectious challenge (FIGS. 50E, 50F; p<0.005 and p=0.05, respectively). Consistent with these results, histologic sections of lung from SPA4 peptide-treated mice exhibited less tissue damage and fewer foci of inflammatory cells as compared to those in untreated, P. aeruginosa infected, control mice (FIG. 50G).

Altogether, these findings show that SPA4 peptide treatment reduces symptoms, bacterial burden, inflammatory cytokine response, and lung inflammation and injury in a mouse model of lung infection. These results demonstrate that SPA4 is a promising therapeutic immunomodulator that can help control lung infection and inflammation.

Discussion of Example 7:

Native SPA exerts its host defense function using several mechanisms, including modulation of TLR4-pathways. The immunomodulatory role of TLR4-interacting regions of SPA has thus been a focus of the presently disclosed inventive concepts. Using a combination of approaches, the SPA4 peptide from the C-terminal region of SPA has been identified that interacts with TLR4. This Example focused on investigating the antibacterial, phagocytic, and anti-inflammatory functions of SPA4 peptide against Gram-negative bacterial stimuli and its biological effects in a mouse model of P. aeruginosa lung infection.

TLR4 recognizes and induces uptake of live Gram-negative bacteria and ligands, and produces an inflammatory response. While a certain level of inflammatory response is required for an effective innate and adaptive immune response, an exaggerated inflammation can cause tissue injury. Endogenous damage-associated molecules, such as hyaluronan and high mobility group box-1 (HMGB-1), released during tissue injury can also serve as TLR4 ligands, further contributing to dysregulated inflammation which aggravates tissue injury. The previous Examples have shown that SPA4 peptide suppressed the LPS-TLR4-induced inflammatory response. However, it was not known whether these findings would translate to therapeutic activity against live Gram-negative bacterial infection in vivo. The results show that SPA4 peptide not only promotes phagocytosis and intracellular trafficking of bacteria to phagolysosomes for lysis and clearance, but it also suppresses the inflammatory response and decreases symptomatology and lung inflammation and injury in an animal model. These findings demonstrate that this dual activity is through SPA4 peptide interaction with TLR4. Binding affinity of SPA4 peptide to recombinant TLR4-MD2 protein is in the nM range indicating relatively strong binding. Pro-phagocytic and anti-inflammatory activity of SPA4 peptide is increased in cells overexpressing TLR4.

Antibiotic therapy is frequently successful in directly killing common pathogens, but in many cases accompanying inflammation and end-organ damage results in significant morbidity and mortality. These problems are exacerbated when antibiotic-resistant organisms are involved. Acute Respiratory Distress Syndrome (ARDS) secondary to lung infection is one such example where lung injury and inflammation worsen the outcomes. While many agents have been tried, no immunomodulators have clearly shown efficacy in this syndrome. The weakness of these therapies appears to be that while they may inhibit inflammation, they also inhibit antimicrobial host defense. Early trials of corticosteroids in ARDS, for example, increased infections. More recent trials using lower doses of corticosteroids improved clinical parameters but did not clearly show any improvement in mortality. This may be due to its inhibition of immune cell functions, including phagocytosis and clearance of pathogens or ligands. An agent which promotes the phagocytic effect or pathogen-clearance, but simultaneously suppresses an inflammatory response, would provide a better option for the treatment of ARDS. In this regard, an attractive target for immunomodulation is TLR4. Several TLR4-immunomodulators are currently being developed as a way to control the inflammation mainly during sepsis. These results show that a TLR4-binding SPA4 peptide has anti-inflammatory properties as well, but also exerts a pro-phagocytic response against Gram-negative bacteria, leading to an increase in bacterial localization in the phagolysosomes (FIG. 46) where lysis and clearance take place. The activity of SPA4 peptide may vary according to the virulence factors expressed by Gram-negative bacteria. The pro-phagocytic effect of SPA4 peptide was more pronounced against P. aeruginosa as compared to those with E. coli. Variations in bacterial cell wall characteristics (capsulated versus non-capsulated, mucoid versus non-mucoid) may have contributed to these differences. A comprehensive study is warranted to further investigate and validate the immunomodulatory effects of the SPA4 peptide against Gram-negative bacteria with different cell wall characteristics and LPS structures.

Unlike full-length SPA, the SPA4 peptide did not directly bind to live bacterial cells (FIG. 48) or LPS. This result, taken together with the demonstrated lack of effect of SPA4 peptide on bacterial growth (FIG. 49), further strengthens the evidence that this peptide acts by host immunomodulation rather than through direct antibacterial effects. SPA4 peptide interaction with TLR4 and its resultant dual pro-phagocytic and anti-inflammatory effects could provide an advantage over the other TLR4-immunomodulators that are currently being developed or are under clinical trials. Most of the TLR4-antagonists are small molecular weight compounds (an antibody: NI0101; a lipid A analog: Eritoran E5564; and small molecules: Resatorvid TAK242 and Ibdilast AV411) (Connolly et al. 2012. Curr Opin Pharmacol 12:510-518). Effects of these molecules on pathogen-uptake, processing and clearance are unknown. While the TLR4-interacting SPA4 peptide may lack the direct bacterial-killing function of native full-length SPA, these results demonstrate that the pro-phagocytic and anti-inflammatory activity of SPA4 peptide through its interaction with TLR4 reduces bacterial burden and inflammation. These effects alleviate the clinical symptoms in a mouse model of bacterial lung infection (FIG. 50).

### Example 8

### Synergistic Effect of SPA-Derived Peptides in Combination with Surfactants

The surfactant CUROSURF® (Chiesi Farmaceutici S.p.A. Corp., Parma, Italy) is a sterile, non-pyrogenic natural porcine pulmonary surfactant consisting of 99% lipids and 1% hydrophobic low-molecular weight surfactant protein (SP)-B and SP-C. CUROSURF® is used for the treatment of Respiratory Distress Syndrome in preterm infants. It does not contain SPA or SP-D. In this Example, it was assessed whether the TLR4-interacting SPA-derived peptides would improve the efficacy of CUROSURF® through their immunomodulatory mechanism.

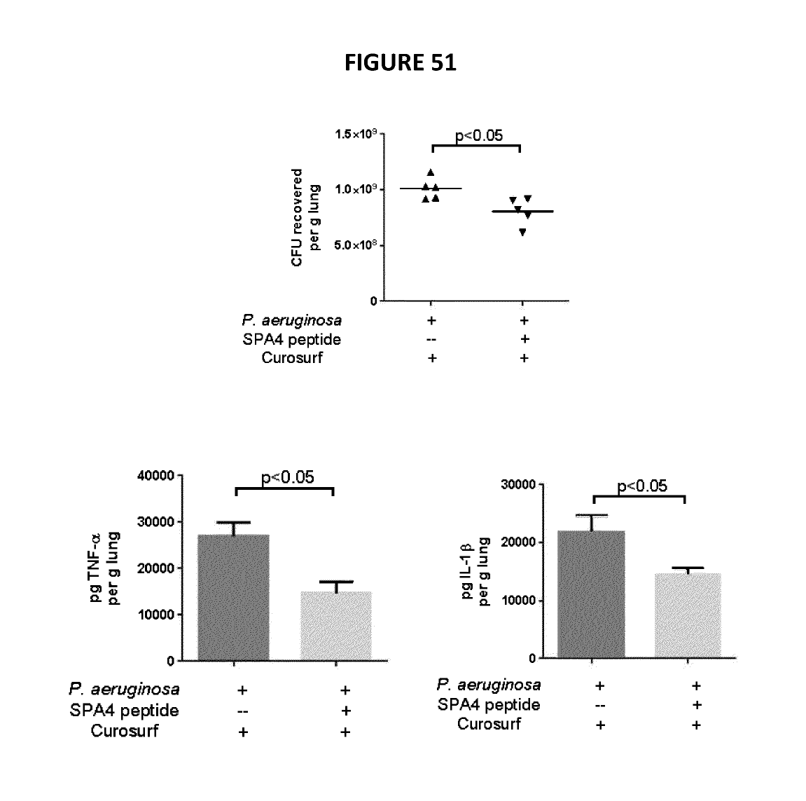

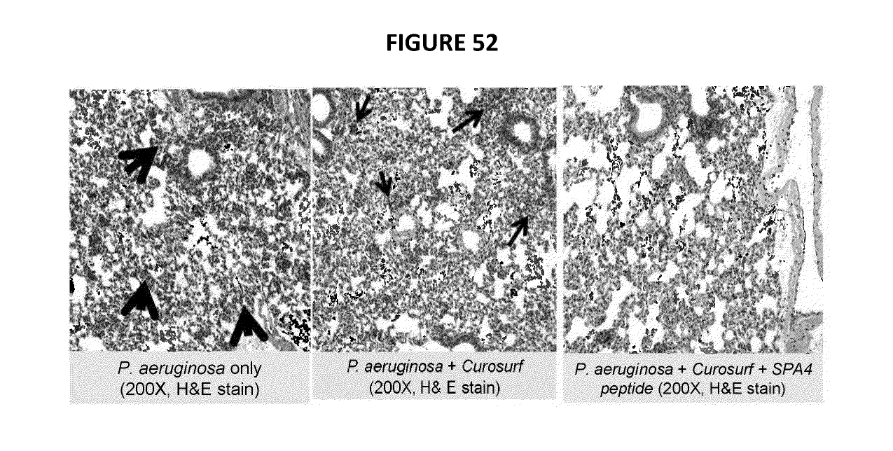

FIGS. 51-52 illustrate experiments performed with CUROSURF® in combination with the SPA4 peptide in a mouse model of bacterial lung infection. In FIG. 51, it is evident that the P. aeruginosa bacterial burden and TNFα and IL-1β cytokine levels in the lung were reduced in the presence of SPA4 peptide, and that these levels were reduced even further in the presence of both SPA4 and CUROSURF®. FIG. 52 contains representative images of H&E stained lung tissue sections after P. aeruginosa challenge and subsequent CUROSURF®+/−SPA4 treatment. As can be seen, similar levels of leukocyte influx were observed in the P. aeruginosa challenged and CUROSURF® treatment groups; however, significantly less lung damage (as evidenced by inflammatory cell infiltrate) was observed in the CUROSURF®+SPA4 treatment group.

While the studies presented in FIGS. 51-52 tested a single treatment dose of CUROSURF® and SPA4 peptide in mice challenged with a single infectious dose of live bacteria over a short period of time, these results indicate that the efficacy of CUROSURF® or similar surfactant products could be improved further by the immunomodulatory activity of the TLR4-interacting SPA4 peptide.

Thus, in accordance with the presently disclosed inventive concepts, there have been provided compositions, as well as methods of producing and using same, that fully satisfy the objectives and advantages set forth hereinabove. Although the presently disclosed inventive concepts have been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the presently disclosed inventive concepts.

1. A method of decreasing the occurrence and/or severity of inflammation associated with a disease condition, wherein Toll-like receptor-4 (TLR4) signaling is involved in the inflammation associated with the disease condition, the method comprising the step of:
administering an effective amount of a composition to a subject suffering from or predisposed to the disease condition, thereby decreasing the occurrence and/or severity of inflammation associated with the disease condition in the subject, wherein the composition comprises a peptide that specifically binds to TLR4, and wherein the peptide comprises at least one of:
(a) a peptide fragment of SEQ ID NO:1, wherein the peptide fragment comprises the motif of SEQ ID NO:2 and is less than 50 amino acids in length;
(b) a peptide having an amino acid sequence of at least one of SEQ ID NOS:3-246;
(c) a peptide having an amino acid sequence that is at least 90% identical to at least one of SEQ ID NOS:3 and 5;
(d) a peptide having an amino acid sequence that differs by two amino acids or less from SEQ ID NO:3 or 5;
(e) a peptide fragment of SEQ ID NO:1, wherein the peptide fragment comprises the amino acid sequence of SEQ ID NO:3 and is less than 50 amino acids in length; and
(f) a peptide comprising amino acids 12-19 of SEQ ID NO:3, wherein the peptide comprises an amino acid sequence that differs from SEQ ID NO:3 by one or two amino acids.

2. The method of claim 1, wherein the disease condition is further defined as a disease condition of the lung.

3. The method of claim 1, wherein the disease condition is further defined as an intestinal disease condition having inflammation associated therewith.

4. The method of claim 1, wherein the disease condition is further defined as a cancer.

5. The method of claim 1, wherein the disease condition is selected from the group consisting of infection-related or non-infectious inflammatory conditions in the lung; asthma, chronic obstructive pulmonary disease (COPD), cystic fibrosis, chronic conditions, pneumonia, acute respiratory distress syndrome (ARDS), bronchopulmonary dysplasia (BPD), and infant respiratory distress syndrome (IRDS); viral, bacterial, and fungal infections; infectious diseases at other mucosal sites; osteoarthritis; GI-associated infection-related or non-infectious inflammatory conditions; infection-related or non-infectious inflammatory conditions in other organs; inflammation-induced cancer; autoimmune diseases; and combinations thereof.

6. The method of claim 1, wherein the isolated peptide fragment or (a) or (e) comprises a portion of the C-terminal carbohydrate recognition domain of SEQ ID NO:1.

7. The method of claim 1, wherein the peptide is less than 40 amino acids in length.

8. The method of claim 1, wherein the peptide is PEGylated.

9. A method of decreasing the occurrence and/or severity of infection in a patient, the method comprising the step of:
administering to the patient a therapeutically effective amount of a pharmaceutical composition, wherein the pharmaceutical composition comprises a peptide that specifically binds to TLR4, and wherein the peptide comprises at least one of:
(a) a peptide fragment of SEQ ID NO:1, wherein the peptide fragment comprises the motif of SEQ ID NO:2 and is less than 50 amino acids in length;
(b) a peptide having an amino acid sequence of at least one of SEQ ID NOS:3-246;
(c) a peptide having an amino acid sequence that is at least 90% identical to at least one of SEQ ID NOS:3 and 5;
(d) a peptide having an amino acid sequence that differs by two amino acids or less from SEQ ID NO:3 or 5;
(e) a peptide fragment of SEQ ID NO:1, wherein the peptide fragment comprises the amino acid sequence of SEQ ID NO:3 and is less than 50 amino acids in length; and
(f) a peptide comprising amino acids 12-19 of SEQ ID NO:3, wherein the peptide comprises an amino acid sequence that differs from SEQ ID NO:3 by one or two amino acids.

10. The method of claim 9, wherein the pharmaceutical composition further comprises at least one additional agent that acts in concert or synergistically with the peptide of the pharmaceutical composition.

11. The method of claim 10, wherein the at least one additional agent is an anti-infective agent selected from the group consisting of aminoglycosides; carbapenems; cephalosporins; glycopeptides; lincosamides; lipopeptides; macrolides; monobactams; nitrofurans; oxazolidonones; polypeptides; quinolones; penicillins; penicillins combined with beta-lactamase inhibitors; sulfonamides; tetracyclines; trimethoprim; sulfamethoxazole and trimethoprim; and combinations and derivatives thereof.

12. The method of claim 10, wherein the at least one additional agent is an anti-inflammatory agent.

13. The method of claim 10, wherein the at least one additional agent is a surfactant.

14. The method of claim 9, wherein the isolated peptide fragment or (a) or (e) comprises a portion of the C-terminal carbohydrate recognition domain of SEQ ID NO:1.

15. The method of claim 9, wherein the peptide is less than 40 amino acids in length.

16. The method of claim 9, wherein the peptide is PEGylated.

17-22. (canceled)

23. A method of promoting lung development and/or function in infants born pre-term, the method comprising the step of:
administering an effective amount of a pharmaceutical composition to an infant subject to promote lung development and/or function and/or maintain immune homeostasis in the infant subject, the pharmaceutical composition comprising a peptide that specifically binds to TLR4, and wherein the peptide comprises at least one of:
(a) a peptide fragment of SEQ ID NO:1, wherein the peptide fragment comprises the motif of SEQ ID NO:2 and is less than 50 amino acids in length;
(b) a peptide having an amino acid sequence of at least one of SEQ ID NOS:3-246;
(c) a peptide having an amino acid sequence that is at least 90% identical to at least one of SEQ ID NOS:3 and 5;
(d) a peptide having an amino acid sequence that differs by two amino acids or less from SEQ ID NO:3 or 5;
(e) a peptide fragment of SEQ ID NO:1, wherein the peptide fragment comprises the amino acid sequence of SEQ ID NO:3 and is less than 50 amino acids in length; and
(f) a peptide comprising amino acids 12-19 of SEQ ID NO:3, wherein the peptide comprises an amino acid sequence that differs from SEQ ID NO:3 by one or two amino acids.

24. The method of claim 23, further comprising the step of administering a surfactant to the subject.

25. The method of claim 23, wherein the isolated peptide fragment or (a) or (e) comprises a portion of the C-terminal carbohydrate recognition domain of SEQ ID NO:1.

26. The method of claim 23, wherein the peptide is less than 40 amino acids in length.

27. The method of claim 23, wherein the peptide is PEGylated.

28-45. (canceled)


SEQUENCE LISTING (261 sequences)

SEQ ID NO: 1 - protein, 248 aa, Homo sapiens
MWLCPLALNLILMAASGAVCEVKDVCVGSPGIPGTPGSHGLPGRDGRDGLKGDPGPPGPMGPPGEMPCPPGNDGLPGAPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 2 - protein, 6 aa, Homo sapiens
  MISC_FEATURE (4)..(4): 3-9 amino acids; each position may contain any amino acid
NYTXRG

SEQ ID NO: 3 - protein, 20 aa, Homo sapiens
GDFRYSDGTPVNYTNWYRGE

SEQ ID NO: 4 - protein, 20 aa, Homo sapiens
VNYTNWYRGEPAGRGKEQCV

SEQ ID NO: 5 - protein, 20 aa, Homo sapiens
YVGLTEGPSPGDFRYSDGTP

SEQ ID NO: 6 - protein, 20 aa, Homo sapiens
GAFRYSDGTPVNYTNWYRGE

SEQ ID NO: 7 - protein, 10 aa, Homo sapiens
GDFRYSDGTP

SEQ ID NO: 8 - protein, 5 aa, Homo sapiens
GDFRY

SEQ ID NO: 9 - protein, 5 aa, Homo sapiens
DFRYS

SEQ ID NO: 10 - protein, 5 aa, Homo sapiens
FRYSD

SEQ ID NO: 11 - protein, 5 aa, Homo sapiens
RYSDG

SEQ ID NO: 12 - protein, 5 aa, homo sapiens
YSDGT

SEQ ID NO: 13 - protein, 5 aa, homo sapiens
SDGTP

SEQ ID NO: 14 - protein, 5 aa, homo sapiens
DGTPV

SEQ ID NO: 15 - protein, 5 aa, homo sapiens
GTPVN

SEQ ID NO: 16 - protein, 5 aa, homo sapiens
TPVNY

SEQ ID NO: 17 - protein, 5 aa, homo sapiens
PVNYT

SEQ ID NO: 18 - protein, 5 aa, homo sapiens
VNYTN

SEQ ID NO: 19 - protein, 5 aa, homo sapiens
NYTNW

SEQ ID NO: 20 - protein, 5 aa, homo sapiens
YTNWY

SEQ ID NO: 21 - protein, 5 aa, homo sapiens
TNWYR

SEQ ID NO: 22 - protein, 5 aa, homo sapiens
NWYRG

SEQ ID NO: 23 - protein, 5 aa, Homo sapiens
WYRGE

SEQ ID NO: 24 - protein, 6 aa, homo sapiens
GDFRYS

SEQ ID NO: 25 - protein, 6 aa, homo sapiens
DFRYSD

SEQ ID NO: 26 - protein, 6 aa, homo sapiens
FRYSDG

SEQ ID NO: 27 - protein, 6 aa, homo sapiens
RYSDGT

SEQ ID NO: 28 - protein, 6 aa, homo sapiens
YSDGTP

SEQ ID NO: 29 - protein, 6 aa, homo sapiens
SDGTPV

SEQ ID NO: 30 - protein, 6 aa, homo sapiens
DGTPVN

SEQ ID NO: 31 - protein, 6 aa, homo sapiens
GTPVNY

SEQ ID NO: 32 - protein, 6 aa, homo sapiens
TPVNYT

SEQ ID NO: 33 - protein, 6 aa, homo sapiens
PVNYTN

SEQ ID NO: 34 - protein, 6 aa, homo sapiens
VNYTNW

SEQ ID NO: 35 - protein, 6 aa, homo sapiens
NYTNWY

SEQ ID NO: 36 - protein, 6 aa, homo sapiens
YTNWYR

SEQ ID NO: 37 - protein, 6 aa, homo sapiens
TNWYRG

SEQ ID NO: 38 - protein, 6 aa, homo sapiens
NWYRGE

SEQ ID NO: 39 - protein, 7 aa, homo sapiens
GDFRYSD

SEQ ID NO: 40 - protein, 7 aa, homo sapiens
DFRYSDG

SEQ ID NO: 41 - protein, 7 aa, homo sapiens
FRYSDGT

SEQ ID NO: 42 - protein, 7 aa, homo sapiens
RYSDGTP

SEQ ID NO: 43 - protein, 7 aa, homo sapiens
YSDGTPV

SEQ ID NO: 44 - protein, 7 aa, homo sapiens
SDGTPVN

SEQ ID NO: 45 - protein, 7 aa, homo sapiens
DGTPVNY

SEQ ID NO: 46 - protein, 7 aa, homo sapiens
GTPVNYT

SEQ ID NO: 47 - protein, 7 aa, homo sapiens
TPVNYTN

SEQ ID NO: 48 - protein, 7 aa, homo sapiens
PVNYTNW

SEQ ID NO: 49 - protein, 7 aa, homo sapiens
VNYTNWY

SEQ ID NO: 50 - protein, 7 aa, homo sapiens
NYTNWYR

SEQ ID NO: 51 - protein, 7 aa, homo sapiens
YTNWYRG

SEQ ID NO: 52 - protein, 7 aa, homo sapiens
TNWYRGE

SEQ ID NO: 53 - protein, 8 aa, homo sapiens
GDFRYSDG

SEQ ID NO: 54 - protein, 8 aa, homo sapiens
DFRYSDGT

SEQ ID NO: 55 - protein, 8 aa, homo sapiens
FRYSDGTP

SEQ ID NO: 56 - protein, 8 aa, homo sapiens
RYSDGTPV

SEQ ID NO: 57 - protein, 8 aa, homo sapiens
YSDGTPVN

SEQ ID NO: 58 - protein, 8 aa, homo sapiens
SDGTPVNY

SEQ ID NO: 59 - protein, 8 aa, homo sapiens
DGTPVNYT

SEQ ID NO: 60 - protein, 8 aa, homo sapiens
GTPVNYTN

SEQ ID NO: 61 - protein, 8 aa, homo sapiens
TPVNYTNW

SEQ ID NO: 62 - protein, 8 aa, homo sapiens
PVNYTNWY

SEQ ID NO: 63 - protein, 8 aa, homo sapiens
VNYTNWYR

SEQ ID NO: 64 - protein, 8 aa, homo sapiens
NYTNWYRG

SEQ ID NO: 65 - protein, 8 aa, homo sapiens
YTNWYRGE

SEQ ID NO: 66 - protein, 9 aa, homo sapiens
GDFRYSDGT

SEQ ID NO: 67 - protein, 9 aa, homo sapiens
DFRYSDGTP

SEQ ID NO: 68 - protein, 9 aa, homo sapiens
FRYSDGTPV

SEQ ID NO: 69 - protein, 9 aa, homo sapiens
RYSDGTPVN

SEQ ID NO: 70 - protein, 9 aa, homo sapiens
YSDGTPVNY

SEQ ID NO: 71 - protein, 9 aa, homo sapiens
SDGTPVNYT

SEQ ID NO: 72 - protein, 9 aa, homo sapiens
DGTPVNYTN

SEQ ID NO: 73 - protein, 9 aa, homo sapiens
GTPVNYTNW

SEQ ID NO: 74 - protein, 9 aa, Homo sapiens
TPVNYTNWY

SEQ ID NO: 75 - protein, 9 aa, homo sapiens
PVNYTNWYR

SEQ ID NO: 76 - protein, 9 aa, homo sapiens
VNYTNWYRG

SEQ ID NO: 77 - protein, 9 aa, homo sapiens
NYTNWYRGE

SEQ ID NO: 78 - protein, 10 aa, homo sapiens
GDFRYSDGTP

SEQ ID NO: 79 - protein, 10 aa, homo sapiens
DFRYSDGTPV

SEQ ID NO: 80 - protein, 10 aa, homo sapiens
FRYSDGTPVN

SEQ ID NO: 81 - protein, 10 aa, homo sapiens
RYSDGTPVNY

SEQ ID NO: 82 - protein, 10 aa, homo sapiens
YSDGTPVNYT

SEQ ID NO: 83 - protein, 10 aa, homo sapiens
SDGTPVNYTN

SEQ ID NO: 84 - protein, 10 aa, homo sapiens
DGTPVNYTNW

SEQ ID NO: 85 - protein, 10 aa, homo sapiens
GTPVNYTNWY

SEQ ID NO: 86 - protein, 10 aa, homo sapiens
TPVNYTNWYR

SEQ ID NO: 87 - protein, 10 aa, homo sapiens
PVNYTNWYRG

SEQ ID NO: 88 - protein, 10 aa, homo sapiens
VNYTNWYRGE

SEQ ID NO: 89 - protein, 11 aa, homo sapiens
GDFRYSDGTPV

SEQ ID NO: 90 - protein, 11 aa, homo sapiens
DFRYSDGTPVN

SEQ ID NO: 91 - protein, 11 aa, homo sapiens
FRYSDGTPVNY

SEQ ID NO: 92 - protein, 11 aa, homo sapiens
RYSDGTPVNYT

SEQ ID NO: 93 - protein, 11 aa, homo sapiens
YSDGTPVNYTN

SEQ ID NO: 94 - protein, 11 aa, homo sapiens
SDGTPVNYTNW

SEQ ID NO: 95 - protein, 11 aa, homo sapiens
DGTPVNYTNWY

SEQ ID NO: 96 - protein, 11 aa, homo sapiens
GTPVNYTNWYR

SEQ ID NO: 97 - protein, 11 aa, homo sapiens
TPVNYTNWYRG

SEQ ID NO: 98 - protein, 11 aa, homo sapiens
PVNYTNWYRGE

SEQ ID NO: 99 - protein, 12 aa, homo sapiens
GDFRYSDGTPVN

SEQ ID NO: 100 - protein, 12 aa, homo sapiens
DFRYSDGTPVNY

SEQ ID NO: 101 - protein, 12 aa, homo sapiens
FRYSDGTPVNYT

SEQ ID NO: 102 - protein, 12 aa, homo sapiens
RYSDGTPVNYTN

SEQ ID NO: 103 - protein, 12 aa, homo sapiens
YSDGTPVNYTNW

SEQ ID NO: 104 - protein, 12 aa, homo sapiens
SDGTPVNYTNWY

SEQ ID NO: 105 - protein, 12 aa, homo sapiens
DGTPVNYTNWYR

SEQ ID NO: 106 - protein, 12 aa, homo sapiens
GTPVNYTNWYRG

SEQ ID NO: 107 - protein, 12 aa, homo sapiens
TPVNYTNWYRGE

SEQ ID NO: 108 - protein, 13 aa, homo sapiens
GDFRYSDGTPVNY

SEQ ID NO: 109 - protein, 13 aa, homo sapiens
DFRYSDGTPVNYT

SEQ ID NO: 110 - protein, 13 aa, homo sapiens
FRYSDGTPVNYTN

SEQ ID NO: 111 - protein, 13 aa, homo sapiens
RYSDGTPVNYTNW

SEQ ID NO: 112 - protein, 13 aa, homo sapiens
YSDGTPVNYTNWY

SEQ ID NO: 113 - protein, 13 aa, homo sapiens
SDGTPVNYTNWYR

SEQ ID NO: 114 - protein, 13 aa, homo sapiens
DGTPVNYTNWYRG

SEQ ID NO: 115 - protein, 13 aa, homo sapiens
GTPVNYTNWYRGE

SEQ ID NO: 116 - protein, 14 aa, homo sapiens
GDFRYSDGTPVNYT

SEQ ID NO: 117 - protein, 14 aa, homo sapiens
DFRYSDGTPVNYTN

SEQ ID NO: 118 - protein, 14 aa, homo sapiens
FRYSDGTPVNYTNW

SEQ ID NO: 119 - protein, 14 aa, homo sapiens
RYSDGTPVNYTNWY

SEQ ID NO: 120 - protein, 14 aa, homo sapiens
YSDGTPVNYTNWYR

SEQ ID NO: 121 - protein, 14 aa, homo sapiens
SDGTPVNYTNWYRG

SEQ ID NO: 122 - protein, 14 aa, homo sapiens
DGTPVNYTNWYRGE

SEQ ID NO: 123 - protein, 15 aa, homo sapiens
GDFRYSDGTPVNYTN

SEQ ID NO: 124 - protein, 15 aa, homo sapiens
DFRYSDGTPVNYTNW

SEQ ID NO: 125 - protein, 15 aa, homo sapiens
FRYSDGTPVNYTNWY

SEQ ID NO: 126 - protein, 15 aa, homo sapiens
RYSDGTPVNYTNWYR

SEQ ID NO: 127 - protein, 15 aa, homo sapiens
YSDGTPVNYTNWYRG

SEQ ID NO: 128 - protein, 15 aa, homo sapiens
SDGTPVNYTNWYRGE

SEQ ID NO: 129 - protein, 16 aa, homo sapiens
GDFRYSDGTPVNYTNW

SEQ ID NO: 130 - protein, 16 aa, homo sapiens
DFRYSDGTPVNYTNWY

SEQ ID NO: 131 - protein, 16 aa, homo sapiens
FRYSDGTPVNYTNWYR

SEQ ID NO: 132 - protein, 16 aa, homo sapiens
RYSDGTPVNYTNWYRG

SEQ ID NO: 133 - protein, 16 aa, homo sapiens
YSDGTPVNYTNWYRGE

SEQ ID NO: 134 - protein, 17 aa, homo sapiens
GDFRYSDGTPVNYTNWY

SEQ ID NO: 135 - protein, 17 aa, homo sapiens
DFRYSDGTPVNYTNWYR

SEQ ID NO: 136 - protein, 17 aa, homo sapiens
FRYSDGTPVNYTNWYRG

SEQ ID NO: 137 - protein, 17 aa, homo sapiens
RYSDGTPVNYTNWYRGE

SEQ ID NO: 138 - protein, 18 aa, homo sapiens
GDFRYSDGTPVNYTNWYR

SEQ ID NO: 139 - protein, 18 aa, homo sapiens
DFRYSDGTPVNYTNWYRG

SEQ ID NO: 140 - protein, 18 aa, homo sapiens
FRYSDGTPVNYTNWYRGE

SEQ ID NO: 141 - protein, 19 aa, homo sapiens
GDFRYSDGTPVNYTNWYRG

SEQ ID NO: 142 - protein, 19 aa, homo sapiens
DFRYSDGTPVNYTNWYRGE

SEQ ID NO: 143 - protein, 5 aa, homo sapiens
YVGLT

SEQ ID NO: 144 - protein, 5 aa, homo sapiens
VGLTE

SEQ ID NO: 145 - protein, 5 aa, homo sapiens
GLTEG

SEQ ID NO: 146 - protein, 5 aa, homo sapiens
LTEGP

SEQ ID NO: 147 - protein, 5 aa, homo sapiens
TEGPS

SEQ ID NO: 148 - protein, 5 aa, homo sapiens
EGPSP

SEQ ID NO: 149 - protein, 5 aa, homo sapiens
GPSPG

SEQ ID NO: 150 - protein, 5 aa, homo sapiens
PSPGD

SEQ ID NO: 151 - protein, 5 aa, homo sapiens
SPGDF

SEQ ID NO: 152 - protein, 5 aa, homo sapiens
PGDFR

SEQ ID NO: 153 - protein, 6 aa, homo sapiens
YVGLTE

SEQ ID NO: 154 - protein, 6 aa, homo sapiens
VGLTEG

SEQ ID NO: 155 - protein, 6 aa, homo sapiens
GLTEGP

SEQ ID NO: 156 - protein, 6 aa, homo sapiens
LTEGPS

SEQ ID NO: 157 - protein, 6 aa, homo sapiens
TEGPSP

SEQ ID NO: 158 - protein, 6 aa, homo sapiens
EGPSPG

SEQ ID NO: 159 - protein, 6 aa, homo sapiens
GPSPGD

SEQ ID NO: 160 - protein, 6 aa, homo sapiens
PSPGDF

SEQ ID NO: 161 - protein, 6 aa, homo sapiens
SPGDFR

SEQ ID NO: 162 - protein, 6 aa, homo sapiens
PGDFRY

SEQ ID NO: 163 - protein, 7 aa, homo sapiens
YVGLTEG

SEQ ID NO: 164 - protein, 7 aa, homo sapiens
VGLTEGP

SEQ ID NO: 165 - protein, 7 aa, homo sapiens
GLTEGPS

SEQ ID NO: 166 - protein, 7 aa, homo sapiens
LTEGPSP

SEQ ID NO: 167 - protein, 7 aa, homo sapiens
TEGPSPG

SEQ ID NO: 168 - protein, 7 aa, homo sapiens
EGPSPGD

SEQ ID NO: 169 - protein, 7 aa, homo sapiens
GPSPGDF

SEQ ID NO: 170 - protein, 7 aa, homo sapiens
PSPGDFR

SEQ ID NO: 171 - protein, 7 aa, homo sapiens
SPGDFRY

SEQ ID NO: 172 - protein, 7 aa, homo sapiens
PGDFRYS

SEQ ID NO: 173 - protein, 8 aa, homo sapiens
YVGLTEGP

SEQ ID NO: 174 - protein, 8 aa, homo sapiens
VGLTEGPS

SEQ ID NO: 175 - protein, 8 aa, homo sapiens
GLTEGPSP

SEQ ID NO: 176 - protein, 8 aa, homo sapiens
LTEGPSPG

SEQ ID NO: 177 - protein, 8 aa, homo sapiens
TEGPSPGD

SEQ ID NO: 178 - protein, 8 aa, homo sapiens
EGPSPGDF

SEQ ID NO: 179 - protein, 8 aa, homo sapiens
GPSPGDFR

SEQ ID NO: 180 - protein, 8 aa, homo sapiens
PSPGDFRY

SEQ ID NO: 181 - protein, 7 aa, homo sapiens
SPGDFRY

SEQ ID NO: 182 - protein, 8 aa, homo sapiens
PGDFRYSD

SEQ ID NO: 183 - protein, 9 aa, homo sapiens
YVGLTEGPS

SEQ ID NO: 184 - protein, 9 aa, homo sapiens
VGLTEGPSP

SEQ ID NO: 185 - protein, 9 aa, homo sapiens
GLTEGPSPG

SEQ ID NO: 186 - protein, 9 aa, homo sapiens
LTEGPSPGD

SEQ ID NO: 187 - protein, 9 aa, homo sapiens
TEGPSPGDF

SEQ ID NO: 188 - protein, 9 aa, homo sapiens
EGPSPGDFR

SEQ ID NO: 189 - protein, 9 aa, homo sapiens
GPSPGDFRY

SEQ ID NO: 190 - protein, 9 aa, homo sapiens
PSPGDFRYS

SEQ ID NO: 191 - protein, 9 aa, homo sapiens
SPGDFRYSD

SEQ ID NO: 192 - protein, 9 aa, homo sapiens
PGDFRYSDG

SEQ ID NO: 193 - protein, 11 aa, homo sapiens
YVGLTEGPSPG

SEQ ID NO: 194 - protein, 11 aa, homo sapiens
VGLTEGPSPGD

SEQ ID NO: 195 - protein, 11 aa, homo sapiens
GLTEGPSPGDF

SEQ ID NO: 196 - protein, 11 aa, homo sapiens
LTEGPSPGDFR

SEQ ID NO: 197 - protein, 11 aa, homo sapiens
TEGPSPGDFRY

SEQ ID NO: 198 - protein, 11 aa, homo sapiens
EGPSPGDFRYS

SEQ ID NO: 199 - protein, 11 aa, homo sapiens
GPSPGDFRYSD

SEQ ID NO: 200 - protein, 11 aa, homo sapiens
PSPGDFRYSDG

SEQ ID NO: 201 - protein, 11 aa, homo sapiens
SPGDFRYSDGT

SEQ ID NO: 202 - protein, 11 aa, homo sapiens
PGDFRYSDGTP

SEQ ID NO: 203 - protein, 12 aa, homo sapiens
YVGLTEGPSPGD

SEQ ID NO: 204 - protein, 12 aa, homo sapiens
VGLTEGPSPGDF

SEQ ID NO: 205 - protein, 12 aa, homo sapiens
GLTEGPSPGDFR

SEQ ID NO: 206 - protein, 12 aa, homo sapiens
LTEGPSPGDFRY

SEQ ID NO: 207 - protein, 12 aa, homo sapiens
TEGPSPGDFRYS

SEQ ID NO: 208 - protein, 12 aa, homo sapiens
EGPSPGDFRYSD

SEQ ID NO: 209 - protein, 12 aa, homo sapiens
GPSPGDFRYSDG

SEQ ID NO: 210 - protein, 12 aa, homo sapiens
PSPGDFRYSDGT

SEQ ID NO: 211 - protein, 12 aa, homo sapiens
SPGDFRYSDGTP

SEQ ID NO: 212 - protein, 13 aa, homo sapiens
YVGLTEGPSPGDF

SEQ ID NO: 213 - protein, 13 aa, homo sapiens
VGLTEGPSPGDFR

SEQ ID NO: 214 - protein, 13 aa, homo sapiens
GLTEGPSPGDFRY

SEQ ID NO: 215 - protein, 13 aa, homo sapiens
LTEGPSPGDFRYS

SEQ ID NO: 216 - protein, 13 aa, homo sapiens
TEGPSPGDFRYSD

SEQ ID NO: 217 - protein, 13 aa, homo sapiens
EGPSPGDFRYSDG

SEQ ID NO: 218 - protein, 13 aa, homo sapiens
GPSPGDFRYSDGT

SEQ ID NO: 219 - protein, 13 aa, homo sapiens
PSPGDFRYSDGTP

SEQ ID NO: 220 - protein, 14 aa, homo sapiens
YVGLTEGPSPGDFR

SEQ ID NO: 221 - protein, 14 aa, homo sapiens
VGLTEGPSPGDFRY

SEQ ID NO: 222 - protein, 14 aa, homo sapiens
GLTEGPSPGDFRYS

SEQ ID NO: 223 - protein, 14 aa, homo sapiens
LTEGPSPGDFRYSD

SEQ ID NO: 224 - protein, 14 aa, homo sapiens
TEGPSPGDFRYSDG

SEQ ID NO: 225 - protein, 14 aa, homo sapiens
EGPSPGDFRYSDGT

SEQ ID NO: 226 - protein, 14 aa, homo sapiens
GPSPGDFRYSDGTP

SEQ ID NO: 227 - protein, 15 aa, homo sapiens
YVGLTEGPSPGDFRY

SEQ ID NO: 228 - protein, 15 aa, homo sapiens
VGLTEGPSPGDFRYS

SEQ ID NO: 229 - protein, 15 aa, homo sapiens
GLTEGPSPGDFRYSD

SEQ ID NO: 230 - protein, 15 aa, homo sapiens
LTEGPSPGDFRYSDG

SEQ ID NO: 231 - protein, 15 aa, homo sapiens
TEGPSPGDFRYSDGT

SEQ ID NO: 232 - protein, 15 aa, homo sapiens
EGPSPGDFRYSDGTP

SEQ ID NO: 233 - protein, 16 aa, homo sapiens
YVGLTEGPSPGDFRYS

SEQ ID NO: 234 - protein, 16 aa, homo sapiens
VGLTEGPSPGDFRYSD

SEQ ID NO: 235 - protein, 16 aa, homo sapiens
GLTEGPSPGDFRYSDG

SEQ ID NO: 236 - protein, 16 aa, homo sapiens
LTEGPSPGDFRYSDGT

SEQ ID NO: 237 - protein, 16 aa, homo sapiens
TEGPSPGDFRYSDGTP

SEQ ID NO: 238 - protein, 17 aa, homo sapiens
YVGLTEGPSPGDFRYSD

SEQ ID NO: 239 - protein, 17 aa, homo sapiens
VGLTEGPSPGDFRYSDG

SEQ ID NO: 240 - protein, 17 aa, homo sapiens
GLTEGPSPGDFRYSDGT

SEQ ID NO: 241 - protein, 17 aa, homo sapiens
LTEGPSPGDFRYSDGTP

SEQ ID NO: 242 - protein, 18 aa, homo sapiens
YVGLTEGPSPGDFRYSDG

SEQ ID NO: 243 - protein, 18 aa, homo sapiens
VGLTEGPSPGDFRYSDGT

SEQ ID NO: 244 - protein, 18 aa, homo sapiens
GLTEGPSPGDFRYSDGTP

SEQ ID NO: 245 - protein, 19 aa, homo sapiens
YVGLTEGPSPGDFRYSDGT

SEQ ID NO: 246 - protein, 19 aa, homo sapiens
VGLTEGPSPGDFRYSDGTP

SEQ ID NO: 247 - protein, 835 aa, Mus musculus
MMPPWLLARTLIMALFFSCLTPGSLNPCIEVVPNITYQCMDQKLSKVPDDIPSSTKNIDLSFNPLKILKSYSFSNFSELQWLDLSRCEIETIEDKAWHGLHHLSNLILTGNPIQSFSPGSFSGLTSLENLVAVETKLASLESFPIGQLITLKKLNVAHNFIHSCKLPAYFSNLTNLVHVDLSYNYIQTITVNDLQFLRENPQVNLSLDMSLNPIDFIQDQAFQGIKLHELTLRGNFNSSNIMKTCLQNLAGLHVHRLILGEFKDERNLEIFEPSIMEGLCDVTIDEFRLTYTNDFSDDIVKFHCLANVSAMSLAGVSIKYLEDVPKHFKWQSLSIIRCQLKQFPTLDLPFLKSLTLTMNKGSISFKKVALPSLSYLDLSRNALSFSGCCSYSDLGTNSLRHLDLSFNGAIIMSANFMGLEELQHLDFQHSTLKRVTEFSAFLSLEKLLYLDISYTNTKIDFDGIFLGLTSLNTLKMAGNSFKDNTLSNVFANTTNLTFLDLSKCQLEQISWGVFDTLHRLQLLNMSHNNLLFLDSSHYNQLYSLSTLDCSFNRIETSKGILQHFPKSLAFFNLTNNSVACICEHQKFLQWVKEQKQFLVNVEQMTCATPVEMNTSLVLDFNNSTCYMYKTIISVSVVSVIVVSTVAFLIYHFYFHLILIAGCKKYSRGESIYDAFVIYSSQNEDWVRNELVKNLEEGVPRFHLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNPLGRHIFWRRLKNALLDGKASNPEQTAEEEQETATWT

SEQ ID NO: 248 - protein, 835 aa, Rattus norvegicus
MMPLLHLAGTLIMALFLSCLRPGSLNPCIEVLPNITYQCMDQNLSKIPHDIPYSTKNLDLSFNPLKILRRYSFSNFSQLQWLDLSRCEIETIEDKAWHGLNQLSTLVLTGNPIKSFSPGSFSGLTNLENLVAVETKMTSLEGFHIGQLISLKKLNVAHNLIHSFKLPEYFSNLTNLEHVDLSYNYIQTISVKDLQFLRENPQVNLSLDLSLNPIDSIQAQAFQGIRLHELTLRSNFNSSNVLKMCLQNMTGLHVHRLILGEFKNERNLESFDRSVMEGLCNVSIDEFRLTYINHFSDDIYNLNCLANISAMSFTGVHIKHIADVPRHFKWQSLSIIRCHLKPFPKLSLPFLKSWTLTTNREDISFGQLALPSLRYLDLSRNAMSFRGCCSYSDFGTNNLKYLDLSFNGVILMSANFMGLEELEYLDFQHSTLKKVTEFSVFLSLEKLLYLDISYTNTKIDFDGIFLGLISLNTLKMAGNSFKDNTLSNVFTNTTNLTFLDLSKCQLEQISRGVFDTLYRLQLLNMSHNNLLFLDPSHYKQLYSLRTLDCSFNRIETSKGILQHFPKSLAVFNLTNNSVACICEYQNFLQWVKDQKMFLVNVEQMKCASPIDMKASLVLDFTNSTCYIYKTIISVSVVSVLVVATVAFLIYHFYFHLILIAGCKKYSRGESIYDAFVIYSSQNEDWVRNELVKNLEEGVPRFQLCLHYRDFIPGVAIAANIIQEGFHKSRKVIVVVSRHFIQSRWCIFEYEIAQTWQFLSSRSGIIFIVLEKVEKSLLRQQVELYRLLSRNTYLEWEDNALGRHIFWRRLKKALLDGKALNPDETSEEEQEATTLT

SEQ ID NO: 249 - protein, 210 aa, Papio hamadryas
DLSSNKIQNIYCKDLQVLHQMPLPNLSLDLSLNPINFIQPGAFKEIRLHKLTLRSNFDDLNVMKTCIQGLAGLEVHRLVLGEFRNERNLEEFDKSALEGLCNLTIEEFRLTYLDYYLDNIIDLFNCLANASSFSLVSVNIKRVEDFSYNFRWQHLELVNCKFEQFPTLELESLKRLTFTANKGGNAFSEVDLPSLEFLDLSRNGLSFKGC

SEQ ID NO: 250 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 251 - protein, 160 aa, Macaca fascicularis
MLPFLFFSTLFSFIFTEAQKHYWVCNSSDASISYTYCDKMQYPISINVNPCIKLKGSRGLLHIFYIPRRDVKQLYFNLYITVNSMTLPKRKEVICRGSDDDYSFCRALKGETVNTTVSFSFKGIKFSKGKYKCVVEAISGSPEEMLFCLEFDIIHQPNSN

SEQ ID NO: 252 - protein, 160 aa, Homo sapiens
MLPFLFFSTLFSSIFTEAQKQYWVCNSSDASISYTYCDKMQYPISINVNPCIELKGSKGLLHIFYIPRRDLKQLYFNLYITVNTMNLPKRKEVICRGSDDDYSFCRALKGETVNTTISFSFKGIKFSKGKYKCVVEAISGSPEEMLFCLEFVILHQPNSN

SEQ ID NO: 253 - protein, 160 aa, Mus msculus
MLPFILFSTLLSPILTESEKQQWFCNSSDAIISYSYCDHLKFPISISSEPCIRLRGTNGFVHVEFIPRGNLKYLYFNLFISVNSIELPKRKEVLCHGHDDDYSFCRALKGETVNTSIPFSFEGILFPKGHYRCVAEAIAGDTEEKLFCLNFTIIHRRDVN

SEQ ID NO: 254 - protein, 160 aa, Rattus norvegicus
MLPFFLFSTLLSPIFTESERQQWICNSSDAIISYSYCDHMKIPISISSEPCIRLKGTNGFVHVEFIPRGNLKNLYFNLFININSIELPKRKEIVCHGYDDDYSFCRALKGEAVNTAIPFSFDGILFPKGHHRCVAEAIAGDTEEKLFCLNFTIIHHHNVN

SEQ ID NO: 255 - protein, 248 aa, Rattus norvegicus
MSLCSLAFTLFLTVVAGIKCNVTDVCAGSPGIPGAPGNHGLPGRDGRDGVKGDPGPPGPMGPPGGMPGLPGRDGLPGAPGAPGERGDKGEPGERGLPGFPAYLDEELQTELYEIKHQILQTMGVLSLQGSMLSVGDKVFSTNGQSVNFDTIKEMCTRAGGNIAVPRTPEENEAIASIAKKYNNYVYLGMIEDQTPGDFHYLDGASVNYTNWYPGEPRGQGKEKCVEMYTDGTWNDRGCLQYRLAVCEF

SEQ ID NO: 256 - protein, 248 aa, Mus musculus
MSLGSLAFTLFLTVVAGIKCNGTEVCAGSPGIPGTPGNHGPPGRDGRDGIKGDPGPPGPMGPPGGMPGLPGRDGLPGAPGAPGEHGDKGEPGERGLPGFPAYLDEELQTASYEIKHQILQTMGVLSLQGSMLSVGDKVFSTNGQSVNFDTIREMCTRAGGHIAAPRNPEENEAIASITKKYNTYPYLGVIEGQTPGDFHYLDGASVNYTNWYPGEPRGRGKEKCVEMYTDGKWNDKGCLQYRLAICEF

SEQ ID NO: 257 - protein, 57 aa, Papio cynocephalus
MWLCPLALTLTLMAASGAACEVKDVCVGSPGIPGTPGSHGLPGRDGRDGVKGDPGPP

SEQ ID NO: 258 - protein, 20 aa, Homo sapiens
EMYTDGQWNDRNCLYSRLTI

SEQ ID NO: 259 - protein, 20 aa, Homo sapiens
PAGRGKEQCVEMYTDGQWND

SEQ ID NO: 260 - protein, 20 aa, Homo sapiens
SFVKKYNTYAYVGLTEGPSP

SEQ ID NO: 261 - protein, 20 aa, Homo sapiens
SLQGSIMTVGEKVFSSNGQS